(19) United States
(12) Patent Application Publication
(10) Pub. No.: US 2022/0040320 A1
(43) Pub. Date: Feb. 10, 2022

(54) ADCS WITH THIOL MULTIPLEX LINKERS

(71) Applicant: Seagen Inc., Bothell, WA (US)

(72) Inventors: Scott Jeffrey, Bothell, WA (US); Kung-Pern Wang, Bothell, WA (US)

(21) Appl. No.: 17/311,508

(22) PCT Filed: Dec. 20, 2019
(86) PCT No.: PCT/US2019/068178

§ 371 Date: Jun. 7, 2021

Related U.S. Application Data
(60) Provisional application No. 62/783,582, filed on Dec. 21, 2018.
(60) Provisional application No. 62/783,707, filed on Dec. 21, 2018.

(51) Int. Cl. A61K 47/68

(52) U.S. Cl. CPC A61K 47/6803; A61K 47/6849

(57) ABSTRACT

The present disclosure provides, inter alia, thiol multiplexed Antibody Drug Conjugates (TM-ADCs) and Multiplex Linking Assemblies (MLAs) that comprise multiple Drug Moieties in a single linking assembly.

### CROSS-REFERENCES TO RELATED APPLICATIONS

This application claims the benefit of priority to U.S. Appl. No. 62/783,707, filed Dec. 21, 2018 and U.S. Appl. No. 62/783,582, filed Dec. 21, 2018, each of which is incorporated by reference in its entirety.

### BACKGROUND OF THE INVENTION

Antibody-drug conjugates (ADCs) combine the tumor targeting specificity of monoclonal antibodies with the potent cell-killing activity of cytotoxic warheads. There has been a surge of interest in designing new ADC formats due in part to the recent clinical success of ADCs, which includes the approvals of brentuximab vedotin (ADCETRIS©) in relapsed Hodgkin lymphoma and anaplastic large-cell lymphoma, and ado-trastuzumab mertansine (KADCYLA©) in HER2-positive metastatic breast cancer. Most of these new methodologies have focused on addressing some of the shortcomings of existing clinical molecules, such as heterogeneous drug loading, limited drug-linker stability, and warheads with activities that are restricted to a subset of cancer types. To enable improved ADCs, much notable advancement has been made in the field. These include site-specific drug-linker conjugation strategies that enable homogeneous loading, drug-linker attachment modalities with improved stability, potent new payloads, and linker strategies that utilize alternative release mechanisms.

Here, we describe an accessible multiplexed drug conjugate technology for native, non-engineered IgGs as well as engineered IgGs, and demonstrate the first use of payload attachments in which high drug loading is achieved via a single linkage chemistry. Depending then on the steric parameters of the payload attachments and utilization of interchain disulfide conjugation as well as conjugation with engineered cysteine residues, ADCs are prepared having enhanced in vitro and in vivo activities compared to conventional ADCs.

### BRIEF SUMMARY OF THE INVENTION

Provided herein are Multiplexer Linking Assembly (MLA) compounds, Thiol Multiplexed Antibody Drug Conjugates (TM-ADCs) comprising an antibody and from one to ten covalently attached Multiplexer Linking Assembly (MLA) Units, wherein each of the one to ten covalently attached Multiplexer Linking Assembly Units is covalently attached to a sulfur atom from a cysteine thiol provided by a reduced interchain disulfide bond of the antibody and/or from engineered cysteine residues introduced into the antibody. Each of the covalently attached Multiplexer Linking Assembly Units has from two to four Drug Moieties (D^{M}) attached thereto and an optional Partitioning Group (Y). Specific embodiments of Multiplexer Linker Assembly (MLA) compounds, which are precursors to MLA Units in a TM-ADC are provided by formula (Ia), shown below:

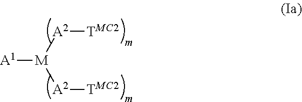

wherein A¹ is a first Linking Group;
each A² is independently a bond or an independently selected second linking group;
each of A¹ and A² comprises 0 or 1 Partitioning Groups (Y) that are covalently attached to the first or second Linking Groups, or a divalent linking component of the first or second Linking Groups;
M is a Multiplexing Group or a first Thiol Multiplexing Group (T^{MC1});
each T^{MC2} is independently a Thiol Multiplexing Group;
the subscript m is 0 or 1; wherein:
when subscript m is 0, M is a first Thiol Multiplexing Group (T^{MC1}) in disulfide form or is a first Thiol Multiplexing Group (T^{MC1}) attached to two Drug Moieties (D^{M}); and when subscript m is 1, each T^{MC2} is in disulfide form, dithiol form with suitable protection of the sulfur atoms or is attached to two Drug Moieties (D^{M}).

Also provided herein are Thiol Multiplexed Antibody Drug Conjugates (TM-ADCs), which are generally prepared using the formula Ia MLA compounds.

In another aspect, provided herein are formula Ia compounds of formula (i) and (ii)

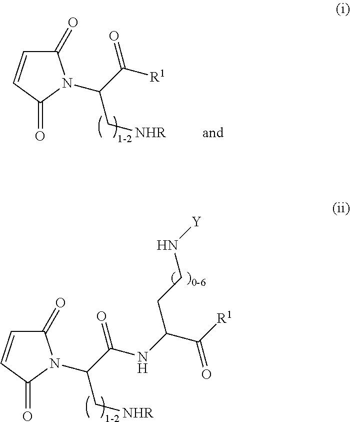

or a salt thereof, wherein R is H or an amino protecting group, and R¹ is selected from the group consisting of:

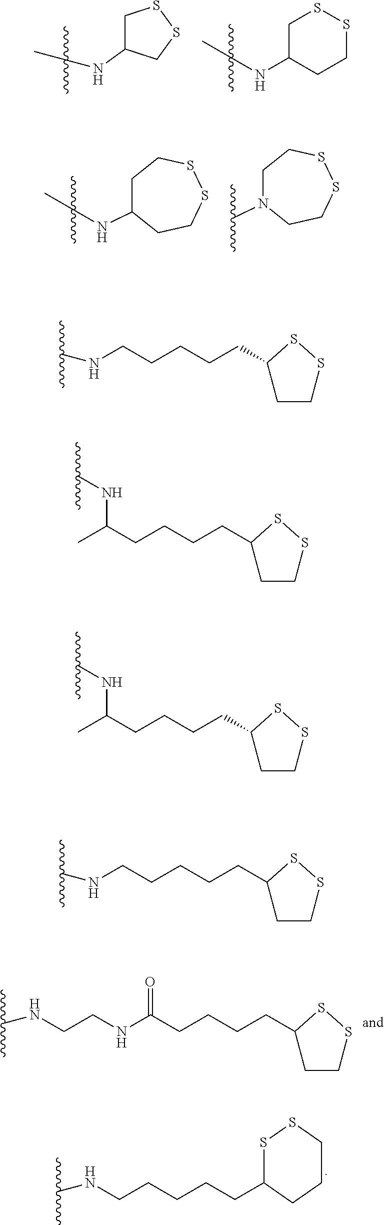

In still other aspects, provided herein are pharmaceutical compositions, and methods for treating diseases, using the described TM-ADCs.

### BRIEF DESCRIPTION OF THE DRAWINGS

FIG. 1 shows an exemplary reaction scheme for preparing a thiol multiplexed antibody drug conjugate (TM-ADC). In the first reaction (left) an antibody thiol from a cysteine residue reacts with a Linking Group (A¹) covalently bound to a Thiol Multiplexing (T^{MC}) Group (an “A-T^{MC}” component) to form an intermediate where the disulfide functional group of the Thiol Multiplexing (T^{MC}) Group is intact. With reference to the second reaction arrow, a reducing agent such as TCEP cleaves the disulfide bond in the Thiol Multiplexing (T^{MC}) Group and the newly formed thiol functional groups are reacted with N-ethyl maleimide (NEM) to form a TM-ADC having a “dummy” Drug Moiety. In this instance, the final product is an N-ethyl maleimide (NEM) capped thiol multiplexed antibody conjugate in which the maleimide moiety has been converted to a thio-substituted succinimide moiety. To prepare a thiol multiplexed antibody drug conjugate (TM-ADC), the desired Drug Moieties (D^{M}) replace NEM in the reaction scheme. Note: the antibody diagram on the right shows the succinimide ring of the A¹ group in a ring opened, or hydrolyzed form. The central antibody diagram may also be in ring-opened form since the resolution of the mass spectrometer is unable to distinguish between a Na+ adduct of product having the ring closed form and a product having the ring opened form.

FIG. 2 provides PLRP chromatography and mass spectroscopy data for 16-load auristatin TM-ADCs obtained from fully reduced cAC10 antibody in which each of the cysteine residues from disulfide bond reduction have been alkylated with a Multiplexer Linker Assembly (MLA) compound of formula A¹-T^{MC} in which A¹ is comprised of a maleimide moiety and the hydrophobicity of the auristatin Drug Units are reduced by PEGylation of the linker component of each Drug Moiety.

FIG. 3 provides PLRP chromatography and mass spectroscopy data for 16-load auristatin TM-ADCs obtained from fully reduced cAC10 antibody in which each of the cysteine residues from disulfide bond reduction have been alkylated with a Multiplexer Linker Assembly compound of formula A¹-T^{MC} in which A¹ is comprised of a maleimide moiety and the hydrophobicity of each Drug Moiety is reduced by a hydrophilic linker component.

FIG. 4A shows an exemplary addition of a A¹-T^{MC} to an antibody thiol from an cysteine residue and subsequent reduction of the disulfide bond in the Thiol Multiplexer (T^{MC}) Group. In this described embodiment, the antibody representation on the right is an abbreviated representation of the chemical structures shown for the middle antibody.

FIG. 4B shows an exemplary reaction scheme for preparing a thiol multiplexed Antibody-Drug Conjugate (TM-ADC) with 4 Drug Moieties (D^{M}). Numbered steps 1 and 2 are similar to those shown in FIG. 4A except that in this instance the reaction is simultaneously performed at 10 different cysteine residues of the antibody. After steps 1 and 2, there are two thiol functional groups in each growing Multiplexer Linking Assembly Unit. Step 3 illustrates the addition of a “A²-T^{MC2}” group to each thiol functional group, followed by step 4 in which the disulfide bond in each T^{MC2} Group is reduced to provide an antibody wherein each Multiplexer Linking Assembly Unit (10 total) displays four thiol functional groups (40 total). In Step 5 a Drug Moiety (D^{M}) is attached to each thiol functional group to form a TM-ADC with 40 Drug Moieties (D^{M}). An exemplary chemical structure for “A2-T^{MC2}” used in step 3 is shown below the reaction scheme.

FIG. 5 illustrates a preparative route for the attachment of eight MLA Units, followed by disulfide bond reduction and Drug Moiety attachments, leading to an ADC having 16 attached Drug Moieties.

FIG. 6 illustrates a preparative route for the attachment of two MLA Units, using introduced modified functional groups capable of participating in Click coupling reactions. In this example, engineered cysteines are covalently modified with a MPr-PEG-azide compound, which then can react with a suitable MLA Group of a Drug Moiety precursor having an alkyne group.

FIG. 7 illustrates a variety of MLA compounds suitable for covalent attachment of two- or four Drug Moeities.

FIG. 8 illustrates the retention time for hydrophilic dendrimeric gemcitabine ADCs with variable drug antibody ratios.

FIG. 9 illustrates the in vitro cytotoxicity of hydrophilic dendrimeric gemcitabine ADCs with variable drug antibody ratios against Hodgkin's lymphoma line L540cy.

### DETAILED DESCRIPTION OF THE INVENTION

### General

Provided herein are Thiol Multiplexed Antibody Drug Conjugates (TM-ADCs) and Multiplexer Linking Assembly (MLA) Units of formula (Ia), (Ib) and (Ic), (IIa), (IIIa) and related subformulae thereof. The TM-ADCs and MLA Units described herein advantageously provide for higher drug loading compared to conventional ADCs within a single linking assembly due to the Multiplexer Linking Assembly Unit. The higher drug loadings provided by a single Multiplexer Linking Assembly are due to the branching nature of the Multiplexer Linking Assembly Units described herein. For example, in some aspects a single Multiplexer Linking Assembly Unit provides for covalent attachment of 2 to 32 or more Drug Moieties (D^{M}) each of which is comprised of Drug Unit (D^{U}), which corresponds in structure to free drug. Thus, the present disclosure provides Multiplexer Linking Assembly Unit with high drug loads that only require a single attachment chemistry to the antibody. When multiple Multiplexer Linking Assembly Units are attached to a single antibody, delivery of a significantly larger dose of free drug is achievable (e.g., 10 Multiplexer Linking Assembly (MLA) Units, each having 4 Drug Moieties attached thereto provides a total of 40 Drug Units).

A important component of the TM-ADCs and Multiplexer Linking Assembly (MLA) Units described herein are the Thiol Multiplexer (T^{MC}) Groups. As described in further detail below, Thiol Multiplexer Groups are components of a Multiplexer Linking Assembly Unit that provide two points of covalent attachment (thiol groups) to Drug Moieties (D^{M}) or Linking Groups (A) comprising an additional T^{MC} group. Thus, the Thiol Multiplexer (T^{MC}) Groups of a Multiplexer Linking Assembly Unit provide antibodies covalently attached to multiple Drug Moieties (D^{M}) within a single linking assembly.

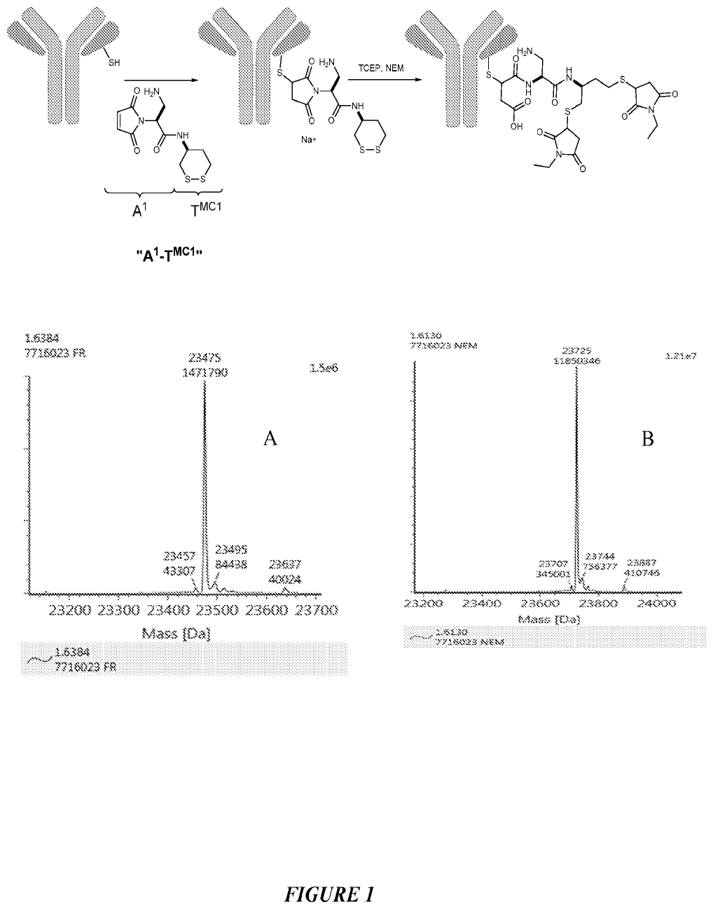

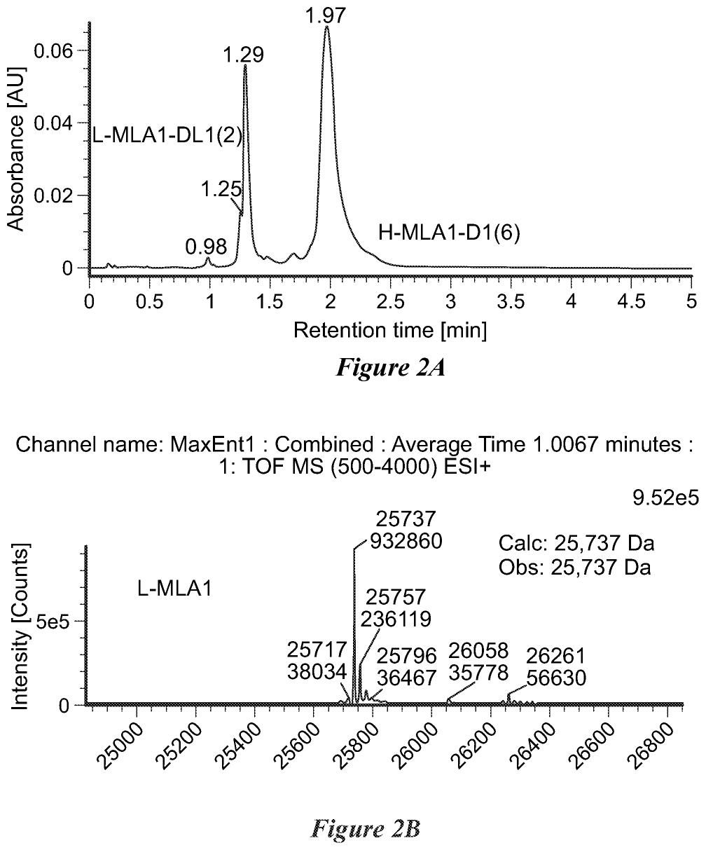

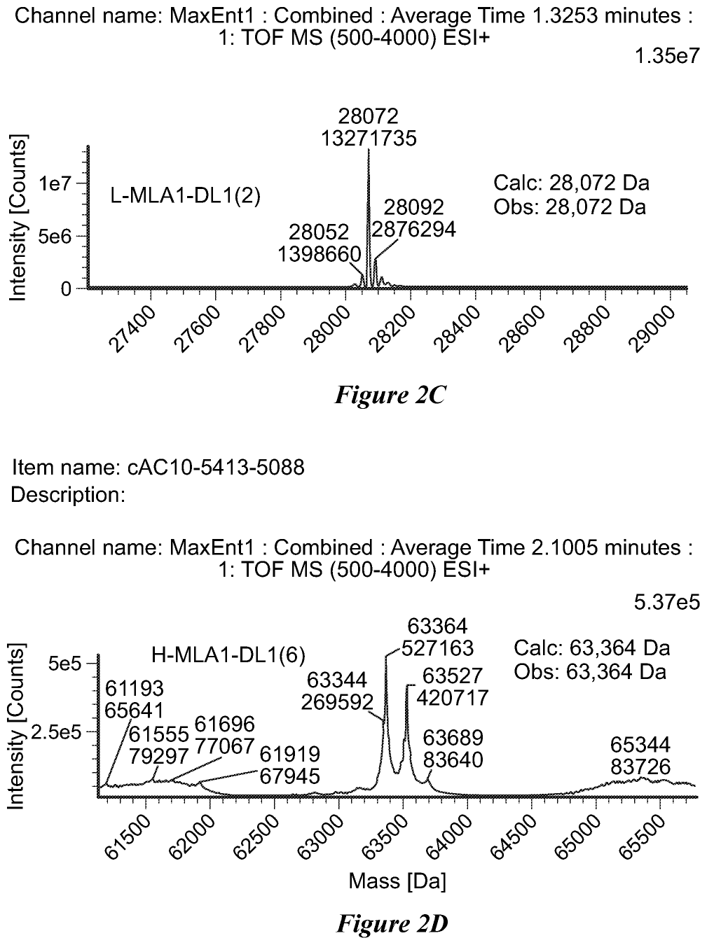

FIG. 1 as well as FIG. 2A and FIG. 2B provide diagrams illustrating how exemplary thiol multiplexed Antibody Drug Conjugates (TM-ADCs) comprising Multiplexer Linking Assembly (MLA) Units are prepared.

### Definitions

Unless stated otherwise, the following terms and phrases as used herein are intended to have the following meanings. When trade names are used herein, the trade name includes the product formulation, the generic drug, and the active pharmaceutical ingredient(s) of the trade name product, unless otherwise indicated by context.

The term “antibody” as used herein is used in the broadest sense and specifically covers intact monoclonal antibodies, polyclonal antibodies, monospecific antibodies, multispecific antibodies (e.g., bispecific antibodies), including intact antibodies and antigen binding antibody fragments, that exhibit the desired biological activity provided that the antigen binding antibody fragments have the requisite number of attachment sites for the desired number of attached drug-linker moieties. The native form of an antibody is a tetramer and consists of two identical pairs of immunoglobulin chains, each pair having one light chain and one heavy chain. In each pair, the light and heavy chain variable regions (VL and VH) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system. (see, e.g., Janeway et al., 2001, Immuno. Biology, 5th Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some aspects, the antibody is of human or murine origin, and in some aspects the antibody is a human, humanized or chimeric antibody.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

An “intact antibody” is one which comprises an antigen-binding variable region as well as a light chain constant domain (C_{L}) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variant thereof.

An “antibody fragment” comprises a portion of an intact antibody, comprising the antigen-binding or variable region thereof. Antibody fragments of the present disclosure include at least one cysteine residue (natural or engineered) that provides a site for attachment of a Multiplexer Linking Assembly. In some embodiments, an antibody fragment includes Fab, Fab′, F(ab′)₂.

An “antigen” is an entity to which an antibody specifically binds.

The terms “specific binding” and “specifically binds” mean that the antibody or antibody fragment thereof will bind, in a selective manner, with its corresponding target antigen and not with a multitude of other antigens. Typically, the antibody or antibody derivative binds with an affinity of at least about 1×10⁻⁷ M, and more typically 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “inhibit” or “inhibition of” means to reduce by a measurable amount, or to prevent entirely (e.g., 100% inhibition).

The term “therapeutically effective amount” refers to an amount of a TM-ADC described herein that is effective to treat a disease or disorder in a mammal. In the case of cancer, the therapeutically effective amount of the conjugate provides one or more of the following biological effects: reduction of the number of cancer cells; reduction of tumor size; inhibition (i.e., slow to some extent and preferably stop) of cancer cell infiltration into peripheral organs; inhibition (i.e., slow to some extent and preferably stop) of tumor metastasis; inhibition, to some extent, of tumor growth; and/or relief to some extent one or more of the symptoms associated with the cancer. To the extent the free drug may release from the TM-ADC to inhibit growth and/or kill existing cancer cells, the free drug is cytostatic and/or cytotoxic. For cancer therapy, efficacy in some aspects is measured by assessing the time to disease progression (TTP) and/or determining the response rate (RR).

Unless otherwise indicated or implied by context, the term “substantial” or “substantially” refers to a majority, i.e. >50% of a population, of a mixture or a sample, typically more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on an Thiol Multiplex Antibody Drug Conjugates (TM-ADC) in which the cellular machinery acts on the TM-ADC or fragment thereof, to intracellularly release the free Drug from the TM-ADC, or other degradant products thereof. The moieties resulting from that metabolic process or reaction are thus intracellular metabolites.

The term “cytotoxic activity” refers to a cell-killing effect of a drug or Thiol Multiplex antibody drug conjugate (TM-ADC) or an intracellular metabolite of a TM-ADC. Cytotoxic activity is typically expressed by an IC₅₀ value, which is the concentration (molar or mass) per unit volume at which half the cells survive exposure to a cytotoxic agent.

The term “cytostatic activity” refers to an anti-proliferative effect other than cell killing of a cytostatic agent, or a TM-ADC having a cytostatic agent as its Drug (D^{M}) or an intracellular metabolite thereof wherein the metabolite is a cytostatic agent.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant or animal origin, including synthetic analogs and derivatives thereof.

The term “cytostatic agent” as used herein refers to a substance that has cytostatic activity e.g., inhibits a function of cells responsible for or that contributes to cell growth or multiplication. Cytostatic agents include inhibitors such as protein inhibitors, e.g., enzyme inhibitors.

The terms “cancer” and “cancerous” refer to or describe the physiological condition or disorder in mammals that is typically characterized by unregulated cell growth. A “tumor” comprises multiple cancerous cells.

An “autoimmune disease” herein is a disease or disorder arising from and directed against an individual's own tissues or proteins.

“Patient” as used herein refers to a subject to which a TM-ADC is administered. Examples of a “patient” include, but are not limited to, a mammal such as a human, rat, mouse, guinea pig, non-human primate, pig, goat, cow, horse, dog, cat, bird and fowl. Typically, a patient is a rat, mouse, dog, non-human primate or human. In some aspects, the patient is a human in need of an effective amount of a TM-ADC.

The terms “treat” or “treatment,” unless otherwise indicated or implied by context, refer to therapeutic treatment and prophylactic measures to prevent relapse, wherein the object is to inhibit or slow down (lessen) an undesired physiological change or disorder, such as, for example, the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment. Those in need of treatment include those already with the condition or disorder and in some aspects further include those prone to have the condition or disorder.

In the context of cancer, the term “treating” includes any or all of: inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

The term “salt,” as used herein, refers to organic or inorganic salts of a compound (e.g., a Drug Moiety (D^{M}), a Multiplexer Linking Assembly (MLA) Unit, or a TM-ADC). In some aspects, the compound contains at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion or other counterion. The counterion may be any organic or inorganic moiety that stabilizes the charge on the parent compound. Furthermore, a salt has one or more than one charged atom in its structure. In instances where there are multiple charged atoms as part of the salt multiple counter ions are sometimes present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Ztrich:Wiley-VCH/VHCA, 2002, the list for which is specifically incorporated by reference herein.

Unless otherwise indicated or implied by context, the term “alkyl” by itself or as part of another term refers to an unsubstituted straight chain or branched, saturated hydrocarbon having the indicated number of carbon atoms (e.g., “—C₁-C₈ alkyl” or “—C₁-C₁₀” alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈ alkyl” groups include, but are not limited to, -methyl, -ethyl, -n-propyl, -n-butyl, -n-pentyl, -n-hexyl, -n-heptyl and -n-octyl; while branched —C₁-C₈ alkyls include, but are not limited to, -isopropyl, -sec-butyl, -isobutyl, -tert-butyl, -isopentyl, and -2-methylbutyl; the term “alkenyl” by itself or as part of another term refers to an unsaturated —C₂-C₈ alkyl and includes, but is not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexylenyl, 2-hexylenyl, and -3-hexylenyl; the term “alkynyl” by itself or as part of another term refers to an unsaturated —C₂-C₈ alkyl having one or more triple bonds, for example, -acetylenyl, -propynyl, -1-butynyl, -2-butynyl, -1-pentynyl, -2-pentynyl and -3-methyl-1 butynyl.

Unless otherwise indicated or implied by context, “alkylene,” by itself of as part of another term, refers to a substituted or unsubstituted saturated or unsaturated branched or straight chain or cyclic hydrocarbon di-radical of the stated number of carbon atoms, typically 1-10 carbon atoms, and having two monovalent radical centers derived by the removal of two hydrogen atoms from the same or two different carbon atoms of a parent alkane. Typical alkylene radicals include but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like. In some aspects, an alkylene is a branched or straight chain hydrocarbon (i.e., it is not a cyclic hydrocarbon). In other aspects, the alkylene is a saturated alkylene that typically is not a cyclic hydrocarbon.

Unless otherwise indicated or implied by context, “aryl,” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated or implied by context, an “arylene,” by itself or as part of another term, is an aryl group as defined above wherein one of the aryl group's hydrogen atoms is replaced with a bond (i.e., it is divalent) and can be in the ortho, meta, or para orientations as shown in the following structures, with phenyl as the exemplary group:

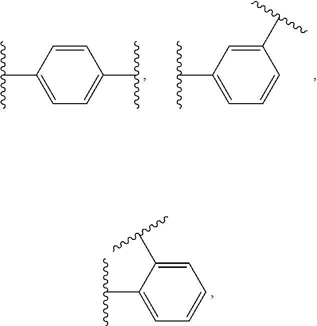

Unless otherwise indicated or implied by context, a “C₃-C₈ heterocycle,” by itself or as part of another term, refers to a monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. In some aspects one or more N, C or S atoms in the heterocycle is oxidized. The ring that includes the heteroatom is in some aspects aromatic and in other aspects nonaromatic. Unless otherwise noted, the heterocycle is attached to its pendant group at any heteroatom or carbon atom that results in a stable structure. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl.

Unless otherwise indicated or implied by context, “C₃-C₈ heterocyclo”, by itself or as part of another term, refers to a C₃-C₈ heterocycle group defined above wherein one of the heterocycle group's hydrogen atoms is replaced with a bond (i.e., it is divalent). In certain aspects, e.g., when a portion of the Multiplexer Linking Assembly comprises a heterocyclo, the heterocyclo is a heterocycle group defined above wherein one or two of the heterocycle group's hydrogen atoms is replaced with a bond (i.e., the heterocyclo is divalent or trivalent).

Unless otherwise indicated or implied by context, a “C₃-C₈ carbocycle,” by itself or as part of another term, is a 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated or implied by context, a “C₃-C₈ carbocyclo”, by itself or as part of another term, refers to a C₃-C₈ carbocycle group defined above wherein another of the carbocycle groups' hydrogen atoms is replaced with a bond (i.e., it is divalent). In certain aspects, e.g., when a portion of the Multiplexer Linking Assembly comprises a carbocyclo, the carbocyclo is a carbocycle group defined above wherein one or two of the carbocycle group's hydrogen atoms is replaced with a bond (i.e., the carbocyclo is divalent or trivalent).

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to ten, preferably one to three, heteroatoms selected from the group consisting of O, N, Si and S, and includes aspects in which a nitrogen and/or sulfur atom is oxidized and aspects in which the nitrogen heteroatom is quaternized. The heteroatom(s) O, N and S is(are) placed at any interior position of the heteroalkyl group and/or at the position at which the alkyl group is attached to the remainder of the molecule. The heteroatom Si is placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CHO—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. In preferred aspects, a C₁ to C₄ heteroalkyl or heteroalkylene has 1 to 4 carbon atoms and 1 or 2 heteroatoms and a C₁ to C₃ heteroalkyl or heteroalkylene has 1 to 3 carbon atoms and 1 or 2 heteroatoms. In other preferred aspects, a heteroalkyl or heteroalkylene is saturated.

Unless otherwise indicated or implied by context, the term “heteroalkylene” by itself or as part of another substituent means a divalent group derived from heteroalkyl (as discussed above), as exemplified by —CH₂—CH₂—S—CH₂—CH₂— and —CH₂—S—CH₂—CH₂—NH—CH₂—. For heteroalkylene groups, in some aspects heteroatoms occupy either or both of the chain termini. Still further, for alkylene and heteroalkylene linking groups, no orientation of the linking group is implied. In certain aspects, e.g., when a Linking Group or Tethering Group comprises a heteroalkylene, the heteroalkylene is a heteroalkyl group defined above wherein one or two of the heteroalkyl group's hydrogen atoms is replaced with a bond (i.e., the heteroalkylene is divalent or trivalent).

“Substituted alkyl” and “substituted aryl” mean alkyl and aryl, respectively, in which one or more hydrogen atoms are each independently replaced with a substituent. Typical substituents include, but are not limited to, —X, −O⁻, —OR, —SR, —S⁻, —NR₂, —NR₃, ═NR, —CX₃, —CN, —OCN, —SCN, —N═C═O, —NCS, —NO, —NO₂, ═N₂, —N₃, —NRC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H₂, —AsO₂H₂, —C(═O)X, —C(═S)R, —CO₂R, —CO₂⁻, —C(═S)OR, C(═O)SR, C(═S)SR, C(═S)NR₂, or C(═NR)NR₂, where each X is independently a halogen: —F, —Cl, —Br, or —I; and each R is independently —H, —C₁-C₂₀ alkyl, —C₆-C₂₀ aryl, —C₃-C₁₄ heterocycle, a protecting group or a prodrug moiety. Typical substituents also include (═O). Alkylene, carbocycle, carbocyclo, arylene, heteroalkyl, heteroalkylene, heterocycle, and heterocyclo groups as described above are unsubstituted or similarly substituted. In some aspects, substituents for “alkyl” and “alkylene” include —X, —O⁻, —OR, —SR, —S⁻, NR₂, CX₃, CN, OCN, SCN, —NRC(═O)R, —C(═O)R, —C(═O)NR₂, —SO₃⁻, —SO₃H, or —CO₂R. In some embodiments, substituents for “aryl” “carbocyclic, “carbocyclo,” “arylene,” “heteroalkyl,” “heteroalkylene,” “heterocycle” and “heterocyclo” include —X, —O⁻, —C₁-C₂₀ alkyl, —OR, —SR, —S, NR₂, CX₃, CN, OCN, —SCN, —NRC(═O)R, —C(═O)R, —C(═O)NR₂, —SO₃⁻, —SO₃H, or —CO₂R, wherein each X is independently —F or —Cl, and R is independently —H or —C₁-C₈ alkyl.

As used herein, the term “free drug” refers to a biologically active species that is not covalently attached either directly or indirectly to any other portion of the TM-ADC, or to a degradant product of a TM-ADC, as a Drug Moiety. Accordingly, free drug refers to the Drug Moiety, as it exists immediately upon cleavage from the Multiplexer Linking Assembly Unit via a release mechanism, which may be provided by the Drug Linker (D^{L}) in the TM-ADC, or to subsequent intracellular conversion or metabolism. In some aspects, the free drug will have the form H-D^{U} or may exist as a charged moiety. The free drug is a pharmacologically active species which is capable of exerting the desired biological effect. In some aspects, the pharamacologically active species is the parent drug and in other aspects includes a component or vestige of a Multiplexer Linking Assembly Unit that has not undergone subsequent intracellular metabolism.

As used herein, the term “Partitioning Group” is a structural unit that masks the hydrophobicity of particular Drug Units (D^{U}) or Multiplexer Linking Assembly (MLA) Units. In some aspects, the Partitioning Group increases the hydrophilic character of a MLA Unit. In other aspects, Partitioning Groups improve the pharmacokinetic properties of a TM-ADC to which they are attached.

As used herein, the term “self-stabilizing linker assembly” refers to substituted succinimide) with a basic functional group proximal to a succinimide capable of catalyzing the hydrolysis of a carbonyl-nitrogen bond of the substituted succinimide. The hydrolysis of a substituted succinimide by the basic functional group forms a self-stabilized linker. Further details of the self-stabilizing linker assembly, which are specifically incorporated by reference herein, are described in WO 2013/173337. In some aspects the self-stabilizing linker assembly is MDPr, which has the structure disclosed herein.

As used herein the term “engineered cysteine residue” or “eCys residue” refers to a cysteine amino acid or a derivative thereof that is incorporated into an antibody. One or more eCys residues can be incorporated into an antibody, and typically, the eCys residues are incorporated in either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Derivatives of cysteine (Cys) include but are not limited to beta-2-Cys, beta-3-Cys, homocysteine, and N-methyl cysteine.

### Aspects and Embodiments

Provided herein are Thiol Multiplexed Antibody Drug Conjugates (TM-ADCs, as described below), as well as Multiplex Linking Assembly (MLA) compounds, whose structures are incorporated into the TM-ADCs and MLA Unit having attached Drug Moieties (D^{M}), which includes Drug Units (D^{U}) if the Drug Moiety contains no Drug Linker (DL) as well as Drug Linkers (D^{L}) having attached Drug Units (D^{U})). Each of the TM-ADCs, the MLA Units or the MLA Units with attached Drug Moieties (D^{M}) will optionally have a Partitioning Group (Y) attached at a site or as part of a Linking Group component of the Thiol Multiplexer (T^{MC}) Group or part of the TM-ADC.

The thiol multiplexing technology described herein readily provides Antibody Drug Conjugates (ADCs) with multiple Drug Moieties attached. Advantageously, antibodies with higher drug loading are able to provide therapeutically effective doses of one or more free drugs which reduces the total amount of antibody to be administered in comparison to conventional ADCs. That is an advantage when the copy number of the targeted antigen is low. Further, the Multiplexer Linking Assembly compounds described herein in preparing the TM-ADCs utilize the well-understood thiol/maleimide chemistry, as well as thiol/haloacetyl chemistry. In each instance, the covalent attachment conditions are mild and well-tolerated by other functional groups either on an antibody or in other portions of the linker assembly itself.

### Multiplexer Linking Assembly

A Multiplexer Linking Assembly (MLA) Unit is characterized by the following features: (1) at least one Linking Group (A′) which provides (i) covalent attachment of MLA to an antibody or an antigen-binding fragment of an antibody, or to an antibody or an antigen-binding fragment of an antibody having a suitable attachment site already available (e.g., an antibody with an attached azide or alkyne component for participation in Click chemistry attachments or Diels-Alder additions) and provides (ii) covalent attachment to a Thiol Multiplexer (T^{MC}) Group or a Multiplexing Group (M); and (2) at least one Thiol Multiplexer (T^{MC}) Group. As described in further detail below, the Thiol Multiplexer (T^{MC}) Group is a moiety that includes two nascent thiol functional groups that are either in a cyclic disulfide form, or in dithiol form in which the sulfur atoms are suitably protected or a branched (non-cyclic) form where the two thiols form thioether linkages to a Drug Moiety (D^{M}) or a further Linking Group (A). Linkage to a further Linking Group (A) provides for additional covalent attachments of other Thiol Multiplexer (T^{MC}) Groups, which in turn provides opportunities for additional drug loading on a single Multiplexer Linking Assembly (MLA) Unit. In some embodiments, the MLA Unit further includes one or more Partitioning Groups (Y) attached thereto. The subscript m denotes the number of a particular group in the compound described herein, for example, in the MLA compound of Formula (I), when subscript m is 1, there are two (A²-T^{MC2}) units present. Each subscript m in a particular compound has the same value. For example, the MLA compound of Formula (I) has two instances of subscript m. Each of those instances is the same: if one subscript m is 0, then the other subscript m is also 0; if one subscript m is 1, then the other subscript m is also 1.

In a principle embodiment, provided herein is a Multiplexer Linking Assembly (MLA) compound, having formula (I):

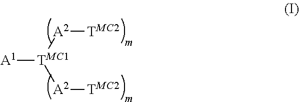

wherein:
A¹ is a first Linking Group;
each A² is independently a bond or an independently selected second Linking Group;
each of A¹ and A² comprises 0 or 1 Partitioning Groups (Y) that are covalently attached to the first or second Linking Groups, or a divalent linking component of the first or second Linking Groups;
M is a Multiplexing Group or a first Thiol Multiplexing Group (T^{MC1});
each T^{MC2} is independently a second Thiol Multiplexing Group;
the subscript m is 0 or 1; wherein:
   when subscript m is 0, M is T^{MC1} in disulfide form or is in suitably protected dithiol form or is a first Thiol Multiplexing Group (T^{MC1}) attached to two Drug Moieties (D^{M}); and when subscript m is 1, each T^{MC2} is in disulfide form or is attached to two Drug Moieties (D^{M}).

In one group of embodiments, provided herein are Multiplexer Linking Assembly (MLA) compounds, having formula (Ia):

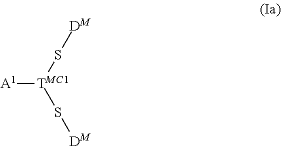

wherein:
A¹ is a Linking Group, further comprising 0 or 1 Partitioning Groups (Y) that are covalently attached to the first Linking Group, or a divalent linking component of the Linking Group;
T^{MC1} is a Thiol Multiplexing Group;
each S is a sulfur atom of the Thiol Multiplexing (T^{MC1}) Group; and
each D^{M} is a Drug Moiety.

In another group of embodiments, provided herein are Multiplexer Linking Assembly (MLA) compounds, having formula (Ib):

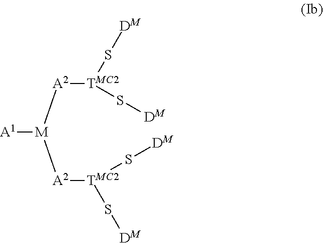

wherein:
A¹ is a first Linking Group;
each A² is independently a bond or an independently selected second linking group, which are the same or different;
each of A¹ and A² comprises 0 or 1 Partitioning Group (Y) that is covalently attached to the first or second Linking Groups, or a divalent linking component of the Linking Group;
M is a Multiplexing Group or a first Thiol Multiplexing (T^{MC1}) Group;
each T^{MC2} is independently a second Thiol Multiplexing Group;
each S is a sulfur atom of the second Thiol Multiplexing (T^{MC2}) Group; and each D^{M} is a Drug Moiety

### Thiol Multiplexer (T^{MC1} and T^{MC2}) Groups

The conceptual approach to the Thiol Multiplexer Groups described herein is shown below with reference to four examples. In each transformation shown, a group that is covalently attached at a single site (shown as ‘a’), is ring closed or ring opened to form two thiols (b) which serve as two sites for further attachments (as in ‘c’). In the compounds and conjugates described herein, the labels T^{MC1} and T^{MC2} are used to refer to the closed ring (or disulfide) form, the suitably protected dithiol form or the trivalent form ‘c’ depending on the MLA compound or TM-ADC. Generally, in a terminal position, the T^{MC} groups will be either in the closed ring (or disulfide) form ‘a’, in ring opened (or dithiol) form b′, or in suitably protected dithiol form, exemplified by b′ or attached to a Drug Moiety (D^{M}) which is either a Drug Unit (D^{U}) or Drug Unit/Drug Linker (D^{U}/D^{L}) combination (and in form ‘c’). Any T^{MC} groups that are not in terminal positions of the Multiplexer Linking Assembly will be in the form ‘c’.

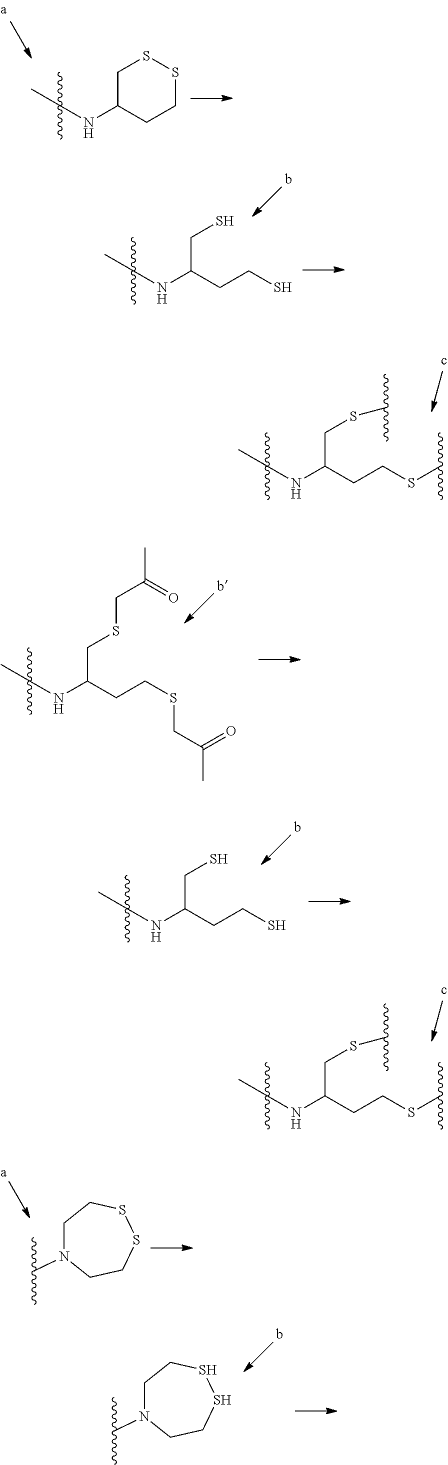

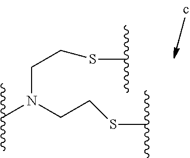 [SMILES: *SCCN(*)CCS*]

As shown in the diagram above, Thiol Multiplexer(T^{MC}) Groups are portions of the Multiplexer Linking Assembly Unit that are present in form ‘a’: a cyclic moiety in the form of a disulfide bond, in form b or b′: an acyclic moiety, or in form ‘c’: a branched linear moiety having two thioether linkages. In general, the disulfide bond of the cyclic form or the suitably protected acyclic form is a precursor to the branched (non-cyclic) moiety having two thioether linkages. The conversion between form a to form b or from form b′ to form is achieved by reducing the disulfide bond, thereby opening the ring and providing two thiol functional groups or reductive removal of the acetamide protecting groups to provide these groups. Each thiol functional group provides a covalent attachment site to either a Drug Moiety (D^{M}) or a Linking Group (e.g. A²) of a further (A-T^{M}c) moiety (as described above).

In addition to the two thiol functional groups, a Thiol Multiplexer (T^{MC}) Group includes a third functional group that provides covalent attachment to a Linking Group (e.g. A¹). The third functional group of the Thiol Multiplexer (T^{MC}) Group depends on the chemical identity of the functional group providing covalent attachment to Thiol Multiplexer (T^{MC}) Group in the Linking Group (A¹, A², etc.). In the diagram above, the third functional group of the Thiol Multiplexer (T^{MC}) Group is a cyclic or exocyclic amine (with reference to form ‘a’ described above). A person of skill in the art will recognize that there are a number of electron donator/electron acceptor pairs that can provide the described covalent attachments. For example, in some embodiments, the third functional group of the Thiol Multiplexers (T^{MC}) is an amino group and the group providing covalent attachment in the Linking Group (A¹, A², etc.) is a carboxylic acid or an ester group. In some embodiments, the third functional group of the Thiol Multiplexers (T^{MC}) is a carboxylic acid or an ester group and the group providing covalent attachment in the Linking Group (A¹, A², etc.) is an amine.

The Thiol Multiplexer Linking Assembly Unit used in the Multiplexer Linking Assembly compounds and Thiol-Multiplexer ADCs, are sometimes based on commercially available components, typically having a five-, six-, seven- or eight-membered carbocyclic ring in which two adjacent ring vertices are replaced by sulfur-forming 1,2-dithiolanes, 1,2-dithianes, 1,2-dithiepanes and 1,2-dithiocanes. In view of the general nature of the ‘multiplexing’ syntheses (see above), the five- and six-membered rings will generally have a functional group external to the ring that is suitable for the synthetic chemistries described herein. In some embodiments the larger seven- and eight-membered rings have an exocyclic functional group that is suitable for the synthetic chemistries described herein, and in other embodiments another ring vertex is replaced with, for example, a nitrogen (amine) which sometimes serves as a functional group in the linking chemistries provided.

Examples of suitable Thiol Multiplexer Groups (in disulfide form) are provided as follows:

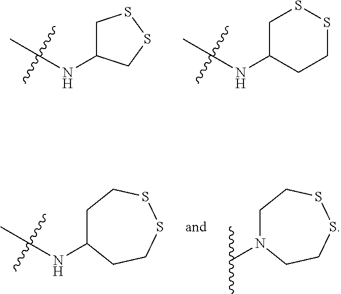 [SMILES: *NC1CSSC1 ; *NC1CCSSC1 ; *NC1CCSSCC1 ; *N1CCSSCC1]

Still other suitable Thiol Multiplexer Groups are based on the following commercially available amines and carboxylic acids.

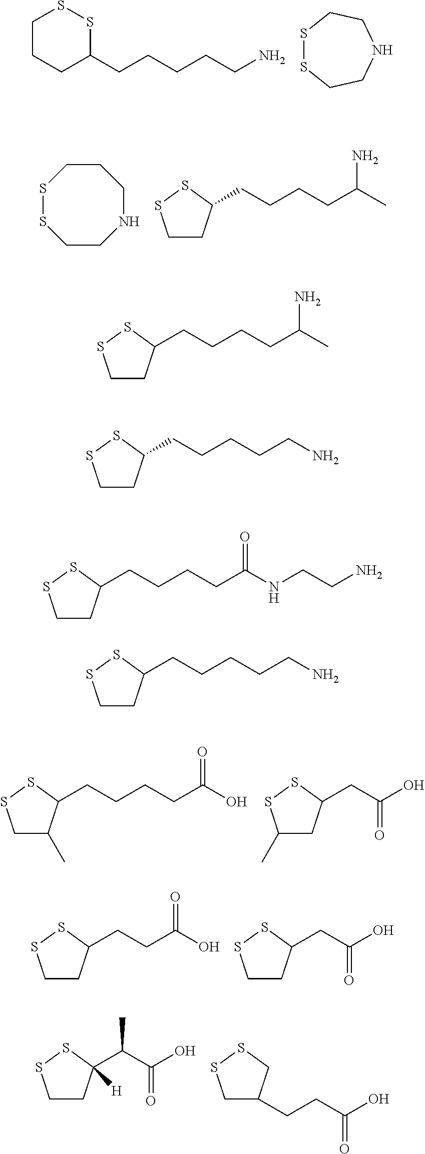

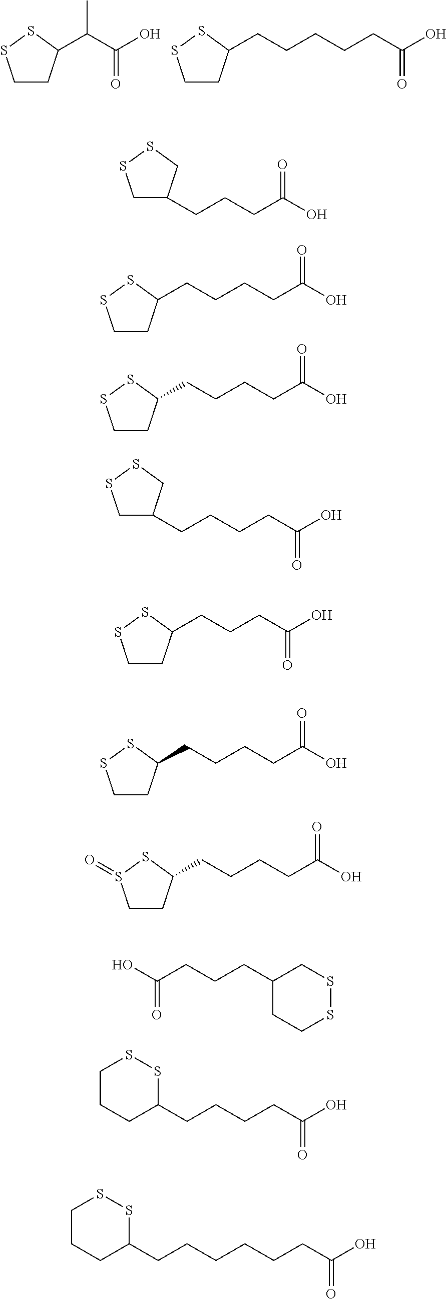

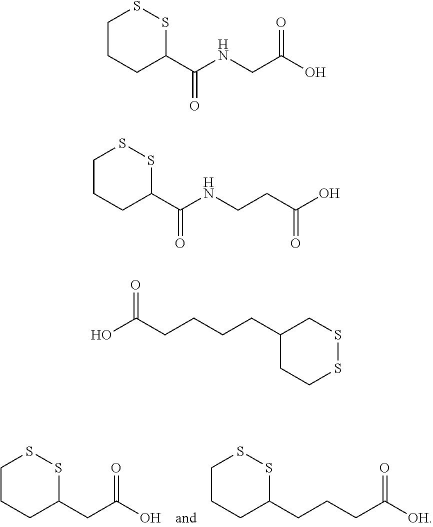 [SMILES: O=C(O)CNC(=O)C1CCCSS1 ; O=C(O)CCNC(=O)C1CCCSS1 ; O=C(O)CCCCC1CCSSC1 ; O=C(O)CC1CCCSS1 ; O=C(O)CCCC1CCCSS1]

### Linking Groups (A¹ and A²)

Linking Groups (A¹ and A²) refer to the portion of the Multiplexer Linking Assembly (MLA) that provides covalent and uniform attachment to antibodies or antibodies having introduced reactive components (for A¹) or other reactive groups on the Multiplexer Group (M) or the Thiol Multiplexer (T^{MC}) Groups in the MLA. In some embodiments, the first Linking Group A¹ comprises a functional group that provides covalent attachment to an antibody cysteine thiol. In other embodiments, the first Linking Group terminates in a component to be used in Click chemistry for attachment to a modified antibody having the compatible Click component. For example, in one embodiment the first Linking Group A¹ terminates in a component having a sufficiently strained alkyne functional group that is reactive towards a modified antibody bearing a suitable azide functional group. Dipolar cycloaddition between the two functional groups in Click Chemistry then results in a triazole heterocyclo. In another embodiment, Diels-Alder type chemistry (4+2 cycloaddition, inverse electron demand) is used for the covalent attachment of a MLA having a terminal 1,2,4,5-tetrazine to a modified antibody bearing a suitable trans cyclooctene functional group. For illustration, general depictions of the Click and Diels-Alder (4+2 cycloaddition) reactions are shown in a) and b) respectively. One of skill in the art will appreciate that a variety of modifications are possible, including, but not limited to, varying the substitution patterns of the reactive components, switching the portion (Ab or MLA) to which each reactive component is attached (in the Click chemistry, the alkyne reactive component could be present on the antibody portion prior to reaction with a MLA bearing an azide, for example).

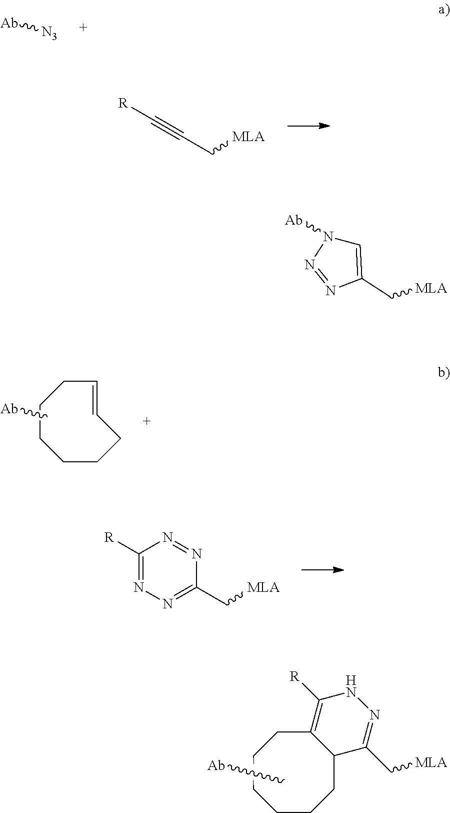

Linking Group A² provides covalent attachment to a reactive moiety of M (or T^{MC1}) which in many instances is a thiol functional group of a Thiol Multiplexer (T^{MC}) Group. In some embodiments, A² is a bond and T^{MC1} is directly linked to T^{MC2} via a covalent bond.

Linking Groups also provide structural separation and include alkylene groups, amino acid groups (for example W_{w} wherein the subscript w is an integer from 0 to 12 and each W is a natural or non-natural amino acid), Partitioning Groups (Y) or other structural components discussed below.

A number of functional groups suitable as thiol Linking Groups have been described in the literature and are well-known for attachment to a thiol moiety. Those functional groups include maleimido moieties (e.g., maleimidocaproyl and self-stabilizing moieties such as mDPR, see WO 2013/173337, which is incorporated by reference herein).

Examples of Linking Groups, prior to covalent attachment to a thiol containing moiety, within the scope of the present disclosure include, groups of Formulas (V) and (VI)

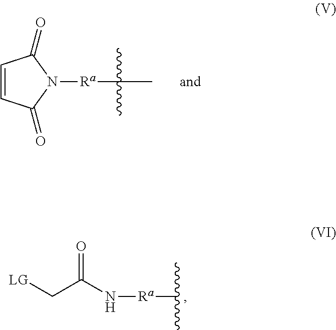

wherein, LG is a leaving group, the wavy line to the right is the point of attachment to a Thiol Multiplexer (T^{MC}) Group or to the remainder of Linking Group (A), and R^{a} is as defined below. One of skill in the art will recognize that the maleimide of Formula (V) is capable of reacting with a cysteine thiol of an antibody to form a thiol-substituted succinimide moiety, optionally in hydrolyzed form, and with reference to Formula VI, the cysteine thiol of an antibody will covalently attach to the carbon bearing LG via nucleophilic attack to displace the leaving group (LG). Suitable leaving groups are well known to one of skill in the art and include halogen, tosylate, and mesylate.

In some embodiments, R^{a} is C₁-C₁₀ alkylene-, C₁-C₁₀ heteroalkylene-, C₃-C₈ carbocyclo-, —O—(C₁-C₈ alkyl)-, -arylene-, C₁-C₁₀ alkylene-arylene-, -arylene-C₁-C₁₀ alkylene-, C₁-C₁₀ alkylene-(C₃-C₈ carbocyclo)-, (C₃-C₈ carbocyclo)-C₁-C₁₀alkylene-, C₃-C₈ heterocyclo-, C₁-C₁₀ alkylene-(C₃-C₈, heterocyclo), (C3-C₈ heterocyclo)-C₁-C₁₀ alkylene-, C₁-C₁₀ alkylene-C(═O)—, C₁-C₁₀ heteroalkylene-C(═O)—, C₃-C₈ carbocyclo-C(═O)—, —O—(C₁-C₈ alkyl)-C(═O)—, -arylene-C(═O), C₁-C₁₀ alkylene-arylene-C(═O)—, -arylene-C₁-C₁₀ alkylene-C(═O)—, C₁-C₈ alkylene-(C₃-C₈, carbocyclo)-C(═O)—, (C₃-C₈ carbocyclo)-C₁-C₁₀ alkylene-C(═O)—, C₃-C₈ heterocyclo-C(═O)—, C₁-C₁₀ alkylene-(C₃-C₈ heterocyclo)-C(═O)—, (C₃-C₈ heterocyclo)-C₁-C₁₀ alkylene-C(═O)—, C₁-C₁₀ alkylene-NH—, C₁-C₁₀ heteroalkylene-NH—, C₃-C₈ carbocyclo-NH—, —O—(C₁-C₈ alkyl)-NH—, -arylene-NH, C₁-C₁₀ alkylene-arylene-NH, -arylene-C₁-C₁₀ alkylene-NH—, C₁-C₁₀ alkylene-(C₃-C₈ carbocyclo)-NH—, (C₃-C₈ carbocyclo)-C₁-C₁₀ alkylene-NH—, C₃-C₈ heterocyclo-NH—, C₁-C₁₀ alkylene-(C₃-C₈ heterocyclo)-NH—, (C₃-C₈ heterocyclo)-C₁-C₁₀ alkylene-NH—, C₁-C₁₀ alkylene-S—, C₁-C₁₀ heteroalkylene-S—, C₃ C₈ carbocyclo-S—, —O—(C₁-C₈ alkyl)-S—, -arylene-S—, C₁-C₁₀ alkylene-arylene-S—, -arylene-C₁-C₁₀ alkylene-S—, C₁-C₁₀ alkylene-(C₃-C₈ carbocyclo)-S—, (C₃-C₈ carbocyclo)-C₁-C₈ alkylene-S—, C₃-C₈ heterocyclo-S—, C₁-C₁₀ alkylene-(C₃-C₈ heterocyclo)-S—, or (C₃-C₈ heterocyclo)-C₁-C₁₀ alkylene-S—. Any of the R¹⁹ substituents can be substituted or non-substituted. In some embodiments, the R¹⁹ substituents are unsubstituted.

In some embodiments, R^{a} is an amino acid or peptide comprising from 2 to 12 natural or unnatural amino acids. In some embodiments, R^{a} is a combination of one or more of the components above with one or more (up to 12 amino acids). In some embodiments, R^{a} is a di-, or tri-peptide. In some embodiments, the amino acids in the peptide unit of R^{a} are independently selected from the group consisting of valine, alanine, β-alanine, glycine, lysine, leucine, and citrulline.

In some embodiments, a Linking Group (A), prior to covalent attachment to a thiol-containing moiety, has formula (VII)

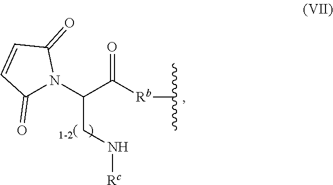

wherein the wavy line to the right is the point of attachment to a Thiol Multiplexer (T^{MC}) Group or to the remainder of Linking Group (A), R^{c} is hydrogen, C₁-C₆ alkyl, C₁-C₆ haloalkyl or a protecting group, and R^{b} is —NH—C₁₋₅alkylene-C(═O)—, or a mono, di-, tri-, tetra-, or penta-peptide. In some embodiments, R^{b} is —NH—CH₂—C(═O)—. In some embodiments, R^{b} is a di-, or tri-peptide. In some embodiments, the amino acids in the peptide unit of R^{b} are independently selected from the group consisting of valine, alanine, β-alanine, glycine, lysine, leucine, and citrulline.

In some embodiments, the R^{a} substituents of Formulas (V) and (VI), are optionally substituted. In some embodiments, the R^{a} substituent of formula (V), is unsubstituted or is substituted by a Basic Unit, e.g. —(CH₂)ₓNHR^{c} or —(CH₂)ₓNR^{c}₂, wherein x is an integer of from 1-4 and each R^{c} is independently selected from the group consisting of H, C₁-C₆ alkyl, and C₁-C₆ haloalkyl, or two R^{c} groups are combined with the nitrogen to which they are attached to form an azetidinyl, pyrrolidinyl or piperidinyl group.

In some embodiments, a Linking Group (A¹ or A²), prior to attachment to a thiol-containing moiety, comprises

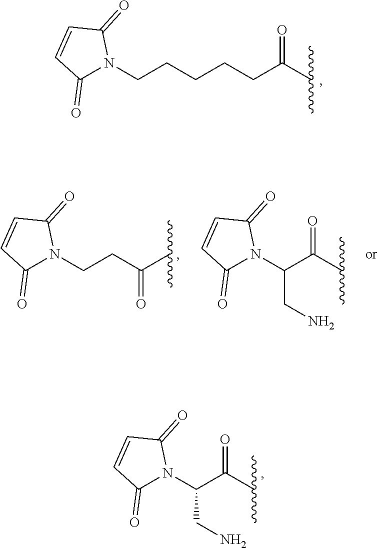

wherein the wavy line to the right is the point of attachment to a Thiol Multiplexer (T^{MC}) Group or to the remainder of Linking Group (A¹ or A²), for example, to an amino acid or di- or tri-peptide.

In some embodiments, Linking Group (A¹ or A²) includes a combination of structural features such as one or more amino acids, one or more polyethylene glycol segments (e.g. PEG₂₄, PEG₁₂, PEG₆, PEG₃), or propanamido units (e.g. —NH—C(O)—CH₂—CH₂—).

Linking Groups (A¹ or A²) of the present disclosure will not always include only linear moieties. For example, in some embodiments, Linking Group (A¹) includes the following moiety:

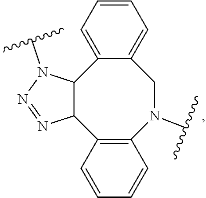 [SMILES: *N1Cc2ccccc2C2C(N=NN2*)c2ccccc21]

wherein the wavy lines on the left and right side of the moiety indicate attachment to the remainder of the Linking Group (A¹).

It is understood that the triazole cyclic group in the paragraph above is formed through an azide-alkyne polar cycloaddition reaction (“click chemistry”) comprising an azide group and

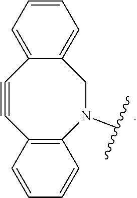 [SMILES: *N1Cc2ccccc2C#Cc2ccccc21]

In some embodiments, one or more Linking Groups (A²) in the MLA are a bond.

In some embodiments, one or more Linking Groups (A¹ or A²) in the MLA include a lysine group. In some embodiments, the amine group on the side chain of the lysine is covalently bound to a Partitioning Group (Y). In some embodiments, the Partitioning Group (Y) covalently bound to the amine group on the side chain of the lysine is a terminally carboxylated polyethelyene glycol group.

In some embodiments, one or more Linking Groups (A¹ or A²) include a di-peptide wherein each amino acid is independently selected from the group consisting glycine, alanine, (3-alanine, valine, leucine, phenylalanine, and proline.

In some embodiments, one or more Linking Groups (A¹ or A²) include a tri-peptide wherein each amino acid is independently selected from the group consisting glycine, alanine, (3-alanine, valine, leucine, phenylalanine, and proline.

In some embodiments, one or more Linking Groups (A¹ or A²) include a mono-, di-, or tri-peptide wherein at least one amino acid selected from the group consisting of aspartic acid, glutamate, lysine, and arginine.

Upon review of this application and the examples provided therein, a person of skill in the art will recognize that the operability of the Multiplexer Linking Assembly compounds and TM-ADCs described herein is not dependent on the exact structure of any one Linking Group (A), and the additional structural features that are not explicitly described herein are capable of being incorporated into one or more Linking Groups (A) without departing from the scope of the present disclosure.

Additionally, one of skill in the art will also appreciate that specific attachment chemistry to an antibody, for example, can alter the synthetic steps leading to a product. In particular, when attachment to a thiol group on an antibody is to be carried out by means of a thiol reactive ‘A’ group, that attachment to the antibody will take place prior to reducing the cyclic thiol multiplexing moieties (T^{MC}) to avoid unwanted or off target reactions between thiols in the linking groups and thiol reactive ‘A’ groups.

Placing the above discussion of Thiol Multiplexers (T^{MC}) and Linking Groups (A) in context, reference is made to the scheme below. In step (1), an “A¹-T^{MC1}” group is attached to an antibody thiol group (typically a thiol produced by reducing interchain disulfide groups or through an introduced (engineered) cysteine moiety. The multiplicity of attached “A¹-T^{MC1}” groups is shown with parentheses and the subscript p, but is not shown in the remainder of the scheme for simplicity. In step (2), the “T^{MC1}” group is reduced, opening the T^{MC1} ring and providing two thiol groups. In step (3), an A²-T^{MC2} group is added by covalently linking the free thiols produced in (1) with a maleimido moiety of A². In step (4) the T^{MC2} groups are reduced as in step (1) to make two free thiol groups. And in step (5), Drug Moieties (D^{M}) are covalently bound to each thiol unit of T^{MC2}.

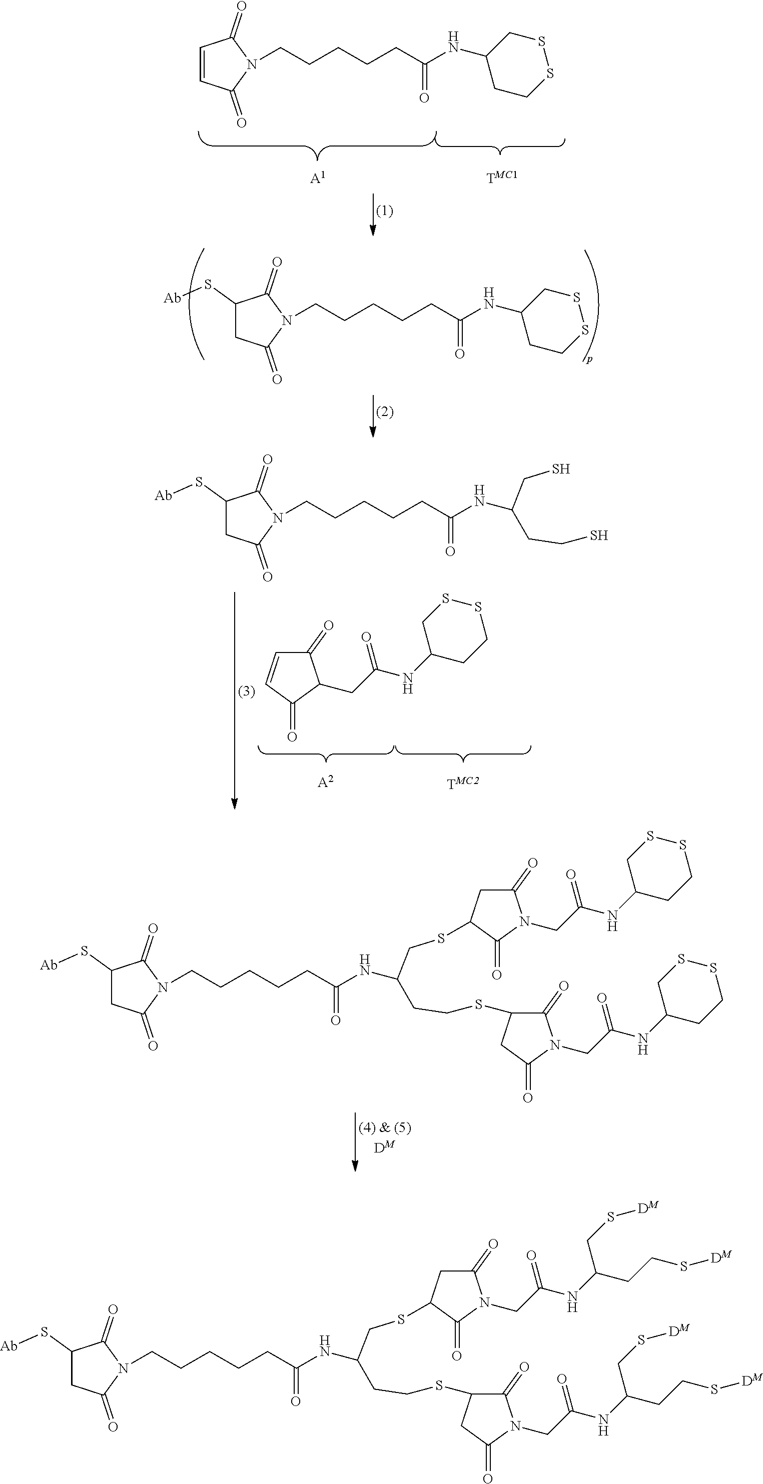

Further illustrations of preparing MLAs of the current disclosure are found in FIG. 1 as well as FIG. 2A and FIG. 2B. One of skill in the art will appreciate that protecting groups can also be employed to carry out construction of the MLA Unit complete with D^{M} attachments prior to the attachment to an antibody. For example, in the scheme below, a protecting group (PG) is used during construction of a MLA compound having four attached Drug Moieties (D^{M}). Removal of PG and the addition of ‘A¹’ (for example, a group having a thiol-reactive maleimide) is then conducted to complete the MLA assembly.

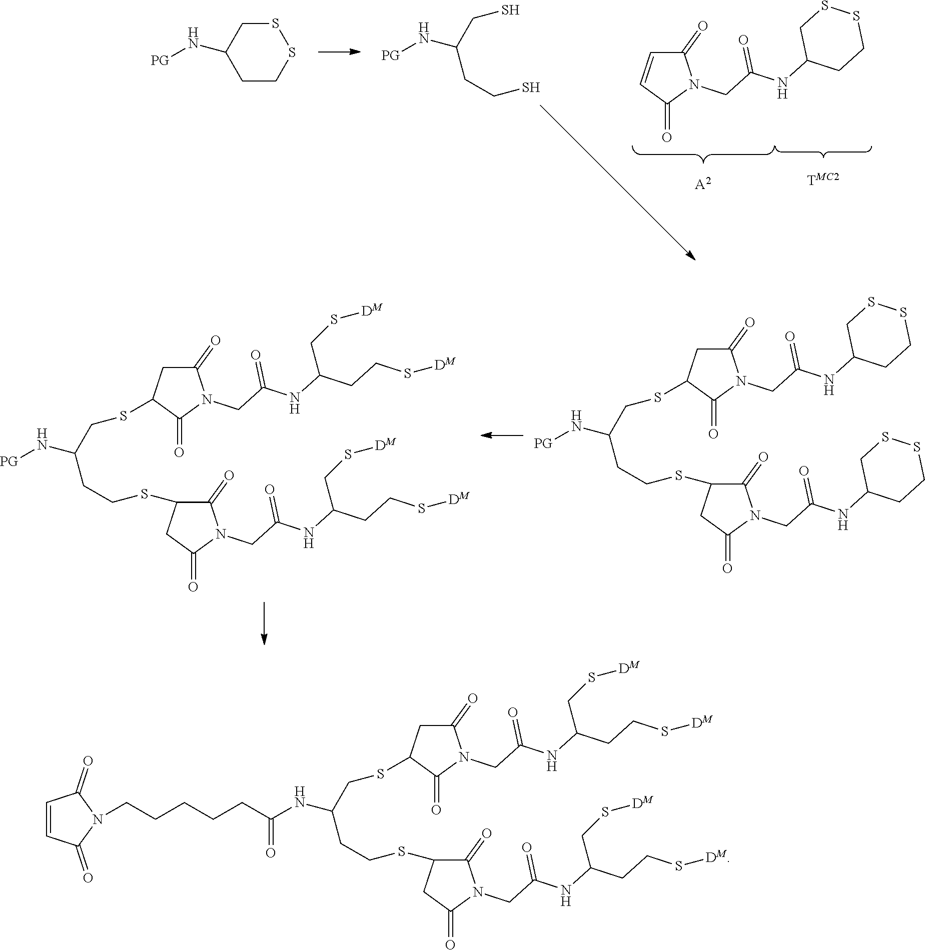

### Drug Moieties (D^{M})

In some embodiments, the MLA compounds are described that have Drug Moieties (D^{M}) attached. Each Drug Moiety (D^{M}) is covalently attached to a sulfur atom of a Thiol Multiplexer (T^{MC}) Group derived from a thiol functional group to form a thioether linkage.

The Drug Moieties (D^{M}) of the present description include a Drug Unit (D^{U}) covalently bound to the Thiol Multiplexer (T^{MC}) via thioether linkage, or a Drug Unit/Drug Linker (D^{U}/D^{L}) combination where the Drug Unit (D^{U}) is covalently bound to the Drug Linker (D^{L}), and the Drug Linker (D^{L}) is covalently bound to the Thiol Multiplexer (T^{MC}) Group via thioether linkage.

### Drug Linkers (D^{L})

Drug Linkers (D^{L}) are included in some embodiments for reasons such as facilitating attachment of the Drug Unit (D^{U}) to the Thiol Multiplexer (T^{MC}), or for introducing a cleavable linking group.

A number of Drug Linkers are known in the art for attachment of Drug Units to functional groups present in antibodies or sites on Assembly Units—and are useful herein for attaching Drug Units (D^{L}) to the Thiol Multiplexers (T^{MC}) of the Multiplexer Linking Assemblies.

In some embodiments, Drug Linkers (D^{L}) include a terminal maleimide, allowing for reliable covalent attachment to each of the Thiol Multiplexer (T^{MC}) Groups. It is understood that the terminal maleimide functional groups are most useful for covalent attachment to Thiol Multiplexers (T^{MC}) Groups comprised of a nucleophilic group such as thiol functional group, in particular an antibody cysteine thiol.

In some embodiments, a Drug Linkers (D^{L}) contains a para-aminobenzoyloxy-carbonyl (PABC) group that is covalently attached to a Drug Unit (D^{U}). In some of those embodiments, the PABC group is substituted with a sugar such as glucose, or a derivative thereof to form a Glucuronide Unit (as described in further detail in WO 2007/011968, which is incorporated by reference herein).

In some embodiments, a Drug Linker (D^{L}) has Formula (VIII) or (IX):

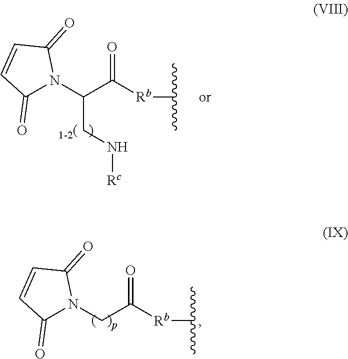

wherein R^{c} is hydrogen or a protecting group, subscript p is an integer from 1-5, and R^{b} is —NH—C₁₋₅alkylene-C(═O)—, —NH—C₁₋₅alkylene-C(═O)—NH—phenylene-CH₂—O—C(═O)—, -(di-peptide)-NH-phenylene-CH₂—O—C(═O)—, or a mono, di-, tri-, tetra-, or penta-peptide. The phenylene in the previously mentioned groups in some embodiments is substituted with a sugar such as glucose, or a derivative thereof to form the Glucuronide Unit. The amine groups of R^{b} in some embodiments will include a methyl (CH₃) in place of H. In some embodiments, R^{b} is a di-, or tri-peptide. In some embodiments, R^{b} is —NH—CH₂—C(═O)—. In some embodiments, the amino acids of the peptide unit in R^{b} are independently selected from the group consisting of valine, alanine, β-alanine, glycine, lysine, leucine, and citrulline. It is understood that the Formulae above are shown before linkage to a Thiol Multiplexer (T^{MC}) Group. The “wavy line” indicates the point of attachment to the Drug Unit (D^{U}). Depending on the Drug Unit and the linking chemistry employed between the Drug Unit (D^{U}) and the Drug Linker (D^{L}), the terminal moiety in the above listed R^{b} groups also in some aspects include a nucleophilic groups such as an amine or a hydroxyl group attached to the terminal carbonyl functional group.

In some embodiments, a Drug Linker (D^{L}) has Formula (VIIIa) or (IXa):

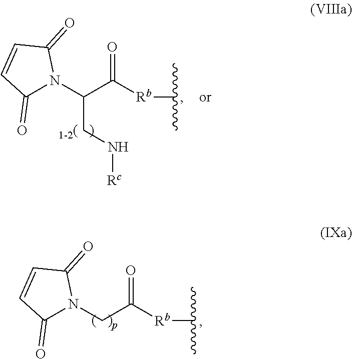

wherein R^{c} is hydrogen or a protecting group, subscript p is an integer from 1-5, and R^{b} is —NH—C₁₋₅alkylene-C(═O)—NH—phenylene-CH₂—O—C(═O)-heterocylyl-C₁₋₄alkylene-b¹-heterocyclyl-b²-; -(di-peptide)-NH-phenylene-CH₂—O—C(═O)-heterocylyl-C₁₋₄alkylene-b¹-heterocyclyl-b²-; wherein b¹ and b² are independently a bond or heteroatoms selected from NH or O, wherein the each heterocyclyl group is a 5 or 6 membered ring having 1-3 heteroatom ring members selected from N, O, and S; and wherein each heterocyclyl group is optionally substituted with from 1 to 3 groups selected from C₁₋₄ alkyl, hydroxyl, alkoxy, carboxyl, and —C(═O)—C₁₋₄ alkyl. In some embodiments, b¹ and b² are each heteroatoms or heteroatom moieties selected from the group consisting of NH and O. The amine groups of R^{b} may also include a methyl (CH₃) in place of H. In some embodiments, R^{b} is a di-, or tri-peptide. In some embodiments, R^{b} is —NH—CH₂—C(═O)—. In some embodiments, the amino acids of the peptide unit in R^{b} are independently selected from the group consisting of valine, alanine, glycine, leucine, and citrulline. It is understood that the Formulae above are shown before covalent attachment to a Thiol Multiplexer (T^{MC}) Group. The “wavy line” indicates the point of attachment to the Drug Unit (D^{U}).

In some embodiments, Drug Linkers (D^{L}) are Releaseable Drug Linkers (D^{RL}) In some other aspects, a Drug Linker (D^{L}) is not a Releaseable Drug Linker. In embodiments without a Releaseable Drug Linker, release of the Drug Unit (D^{U}) is via a total protein degradation pathway (i.e., non-cleavable pathway).

For those embodiments in which the Drug Linker (D^{L}) is a Releaseable Drug Linker (D^{RL}), that group allows efficient release of free drug at the targeted cell, sufficient to exert, e.g., an antiproliferative effect. Preferably, the Releaseable Drug Linker (D^{RL}) is designed for efficient release of the free drug once the TM-ADC has been internalized into the target cell, but may also be designed to release free drug within the vicinity of targeted cells. Suitable recognition sites for cleavage are those that allow efficient release of a TM-ADC's Drug Unit(s). Preferably, the recognition site is a peptide cleavage site (such as in a peptide-based releasable linker assembly), a sugar cleavage site (such as in sugar-based releasable linker assembly, which is or is comprised of a Glucuronide Unit as described in WO 2007/011968), or a disulfide cleavage site (such as in disulfide-based releasable linker assembly). Examples of peptide cleavage sites include those recognized by intracellular proteases, such as those present is lysosomes. Examples of sugar cleavage sites include those recognized by glycosidases, including glucuronidases, such as beta-glucuronidase.

In some embodiments, each Releaseable Drug Linker (D^{RL}) is a di-peptide. In some embodiments, the di-peptide is -Val-Cit-, -Phe-Lys- or -Val-Ala-.

In some embodiments, each Releaseable Drug Linker (D^{RL}) is independently selected from the group consisting of maleimido-caproyl (mc), maleimido-caproyl-valine-citrulline (mc-vc), maleimido-caproyl-valine-citrulline-paraaminobenzyloxycarbonyl (mc-vc-PABC) and MDPr-vc. It is understood that D^{RL} in some embodiments is further substituted with a basic moiety such as an aminoalkyl, which is an exemplary Basic Unit, to form a self-stabilizing succinimide linker discussed above and in greater detail in WO 2013/173337.

In some selected embodiments, the Drug Linker (D^{L}), prior to attachment to an antibody thiol is, for example, a maleimido-containing linkers that is cleavable by a protease. Accordingly, exemplary D^{L} groups cleavable by a protease for use with the TM-ADCs described herein include the following wherein S is from a thiol functional group of a Thiol Multiplexer (T^{MC}) Group, the wavy line to the right is an Drug Unit (D^{U}), and the wavy line to the left is a Thiol Multiplexer (T^{MC}):

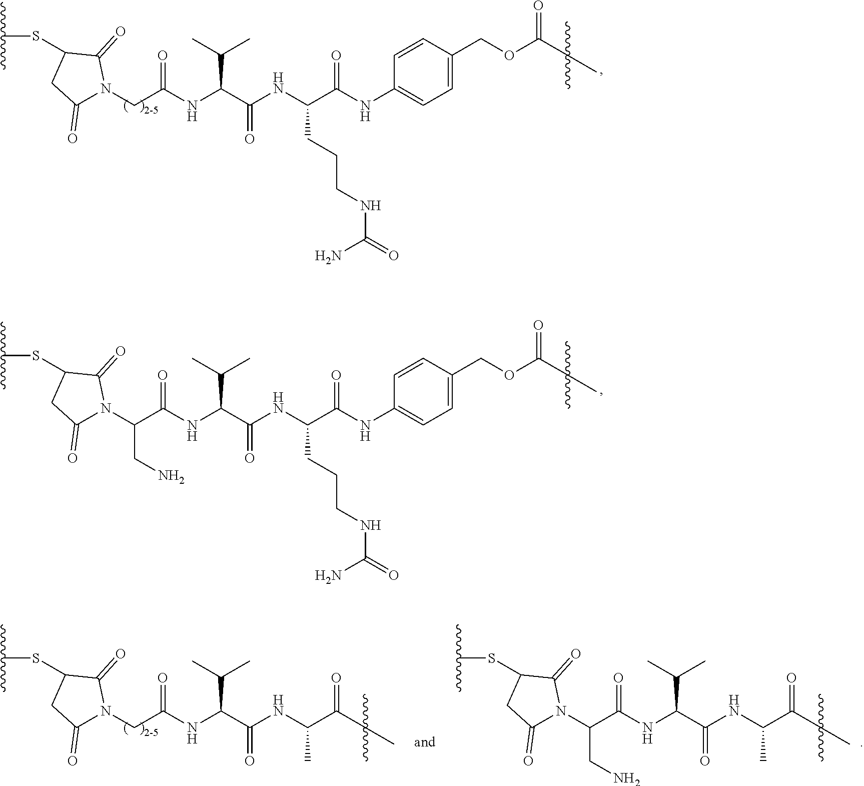

General methods of covalent attachment of a Drug Unit (D^{U}) to an Drug Linker (D^{L}) are known in the art and linkers known in the art for traditional ADCs may be used with the TM-ADCs of the present disclosure. For example, auristatin and maytansine ADCs are currently in clinical development for the treatment of cancer. Monomethyl auristatin E is conjugated through a protease cleavable peptide linker to an antibody, monomethyl auristatin F is conjugated directly to an antibody through maleimidocaproic acid residue, the maytansine DM1 is conjugated through a disulfide or directly through the heterobifunctional SMCC linker, and maytansine DM4 is conjugated through a disulfide linker. In preferred embodiment those linker systems are used with the TM-ADCs described herein and provide release of free drug by an enzymatically cleavable or non-enzymatically cleavable system depending on the linker system used.

Disulfide, thioether, peptide, hydrazine, ester, or carbamate bonds are all examples of bonds that are also useful for connecting Drug Unit (D^{U}) to a Drug Linker (D^{L}).

Optional Partitioning Groups (Y) can be linked via any suitable atom of the Drug Linker (D^{L}). Methods of making such linkages are known in the art.

### Drug Unit (D^{U})

As discussed above, Drug Units (D^{U}) are covalently attached to the MLA Unit via a Thiol Multiplexer (T^{MC}) Group or attached to the MLA Unit via a Drug Linker (D^{L}). It is understood that the Drug Linker (D^{L}) may either be attached to the Drug Unit (D^{U}) prior to MLA Unit attachment, or to the MLA prior to Drug Unit (D^{U}) attachment.

In some embodiments, the Drug Units (D^{U}) are Drugs having cellular cytotoxic activities ranging from 1 to 100 nM. There are a number of different assays that can be used for determining whether a TM-ADC exerts a cytostatic or cytotoxic effect on a cell line. In one example for determining whether a TM-ADC exerts a cytostatic or cytotoxic effect on a cell line, a thymidine incorporation assay is used. For example, cells at a density of 5,000 cells/well of a 96-well plated is cultured for a 72-hour period and exposed to 0.5 μCi of ³H-thymidine during the final 8 hours of the 72-hour period, and the incorporation of ³H-thymidine into cells of the culture is measured in the presence and absence of TM-ADC. The TM-ADC has a cytostatic or cytotoxic effect on the cell line if the cells of the culture have reduced ³H-thymidine incorporation compared to cells of the same cell line cultured under the same conditions but not contacted with the TM-ADC.

In another example, for determining whether a TM-ADC exerts a cytostatic or cytotoxic effect on a cell line, cell viability is measured by determining in a cell the uptake of a dye such as neutral red, trypan blue, or ALAMAR™ blue (see, e.g., Page et al., 1993, Intl. J of Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Nat'l Cancer Inst. 82:1107-12). Preferred TM-ADCs include those with an IC₅₀ value (defined as the mAB concentration that gives 50% cell kill) of less than 1000 ng/ml, preferably less than 500 ng/ml, more preferably less than 100 ng/ml, even most preferably less than 50 or even less than 10 ng/ml on the cell line.

In some embodiments, the Drug Units are those having cellular potencies that would not be expected to provide active ADCs in vitro when conjugated at 8 or less drugs/mAb. In some embodiments the Drug Unit is incorporates a drug that is not hydrophobic or has a cLogP of <2.5. In some embodiments, the drug has a cLogP of between about 0 and about 2.5, between about 0 and 2, between about 0 and about 1.5, between about 0 and about 1, or between about 0 and about 0.5. In some embodiments the Drug Unit incorporates a drug having more hydrophilic properties—for example, a Drug Unit having a cLogP of <1.0. In some embodiments, the drug has a cLogP of between about 0 and about 1, for example, about 0, about 0.1, about 0.2, about 0.3, about 0.4, about 0.5, about 0.6, about 0.7, about 0.8, about 0.9, or about 1.

In some embodiments, the Drug Units are those drugs possessing charged residues (acids, amines, phosphates), sugars, or poly hydroxylated groups. In some embodiments, the Drug Units are selected from nucleoside analogs; HDAC inhibitors; anthracyclines; NAMPT inhibitors; hydrophilic prodrugs; SN-38 glucuronide, etoposide phosphate; low molecular weight drugs (e.g., drugs having a molecular weight less than about 600); nitrogen mustards (melphalan); and proteosome inhibitors (lenalidomide).

In some embodiments, the Drug Unit is selected from pyrimidine antagonists and purine antagonists. In some embodiments, the Drug Unit is selected from antimetabolites such as antifolates: Methotrexate, Pemetrexed, L-leucovorin, GW1843, Raltitrexid, ZD9331, Pralatrexate, and Lometrexol.

The Drug Unit (D^{U}) will typically correspond in structure to a cytotoxic, cytostatic or immunosuppressive drug, also referred to herein as a cytotoxic, cytostatic or immunosuppressive agent. In some embodiments, the free drug that is incorporated into a Drug Unit (D^{U}) has an atom that can form a bond with a Thiol Multiplexer (T^{MC}) Group. In other embodiments, the free drug that is incorporated into a Drug Unit (D^{U}) has a carboxylic acid or ester that can form a bond with a Thiol Multiplexer (T^{MC}).

In some embodiments, the Drug Unit (D^{U}) has an atom that forms a bond with a Drug Linker (D^{L}). In some embodiments, the Drug Unit (D^{U}) has a nitrogen atom that forms a bond with a Drug Linker (D^{L}). In other embodiments, the Drug Unit (D^{U}) has a carboxylic acid residue that forms a bond with a Drug Linker (D^{L}). In other embodiments, the Drug Unit (D^{U}) has a sulfur atom from a thiol functional group of a free drug that forms a bond with a Drug Linker (D^{L}). In still other embodiments, the Drug Unit (D^{U}) has a heteroatom from a hydroxyl group of a free drug or is from alcohol-containing free drug, or has a carbonyl functional group from the free drug that forms a bond with a Drug Linker (D^{L}).

The Drug Unit (D^{U}) preferably incorporates a hydrophilic drug or a moderately hydrophobic drug so as to accommodate the higher loading achievable by the present invention. If the drug is too hydrophobic an undesirable amount of aggregation may occur in the resulting TM-ADC, but in certain instances may be ameliorated to an acceptable extent by incorporation of a Partitioning Group into the Multiplexer Linking Assemblies and/or by use of a hydrophilic Linker Group (A) and/or a hydrophilic Drug Linker (D^{L}), in particular, ones that exist substantially in ionized from at physiological pH. An exemplary hydrophilic Linker Group (A) is comprised of a self-stabilizing moiety, which contain a succinimide moiety in hydrolyzed form and Basic Unit (BU). Self-stabilizing moieties are hydrophilic due to BU having an amine in protonated form and the hydrolyze succinimide moiety displaying a carboxylate anion. An exemplary hydrophilic Drug Linker is comprised of a Glucuronide Unit in which the sugar is glucuronic acid. A moderately hydrophobic to hydrophilic drug has a ClogP of 2.5 or less and/or a polar surface area of 80 angstroms squared or more. In some embodiments, drugs to be used in the present invention will have a ClogP value of 2.5 or less, 2.0 or less, 1.5 or less, 1.0 or less, 0.5 or less 0 or less, -0.5 or less or -1.0 or less. In other embodiments free drugs to be used as described herein will have a polar surface are of about 80 angstroms squared or more, about 90 angstroms squared or more about 100 angstroms squared or more, 110 angstroms squared or more or 120 angstroms squared or more. In some embodiments, the drugs to be used as described herein will have a polar surface are of about 80 angstroms squared to about 140 angstroms squared, or any value in between. For example, about 80 angstroms squared, about 90 angstroms squared, about 100 angstroms squared, about 80 angstroms squared, about 110 angstroms squared, about 120 angstroms squared, about 130 angstroms squared, about 140 angstroms squared, or any value in between.

General procedures for linking a drug to linkers are known in the art. See, for example, U.S. Pat. Nos. 8,163,888, 7,659,241, 7,498,298, U.S. Publication No. US20110256157 and International Application Nos. WO2011023883, and WO2005112919, each of which is incorporated by reference herein, particularly in regards to the aforementioned general procedures.

### Partitioning Groups (Y)

The MLAs and TM-ADCs described herein in certain embodiments include attached Partitioning Groups (Y). The Partitioning Groups are useful, for example, to mask the hydrophobicity of particular Drug Units or Multiplexer Linking Assemblies. Accordingly, a number of Partitioning Groups will act to increase the hydrophilic character of the TM-ADC to which they are attached to reduce aggregation of the ADCs, which may occur at the highest drug loading for moderately hydrophic drugs, which have a ClogP of between about 2.5 to about 1 or have a polar surface area of between about 80 to about 100 angstroms squared.

Representative Partitioning Groups include polyethylene glycol (PEG) units, cyclodextrin units, polyamides, hydrophilic peptides, polysaccharides and dendrimers. In some embodiments, the Partitioning Group Y comprises a polyethylene glycol group.

When the Partitioning Group is included in one or more of groups A, D^{M}, M, T^{MC}, the group may include a lysine residue which provides simple functional conjugation of the Partitioning Group to the Multiplexer Linking Assembly.

### Polyethylene Glycol Unit (PEG)

Polydisperse PEGS, monodisperse PEGS and discrete PEGs can be used to make the Compounds of the present invention. Polydisperse PEGs are a heterogeneous mixture of sizes and molecular weights whereas monodisperse PEGs are typically purified from heterogeneous mixtures and are therefore provide a single chain length and molecular weight. Preferred PEG Units are discrete PEGs, compounds that are synthesized in step-wise fashion and not via a polymerization process. Discrete PEGs provide a single molecule with defined and specified chain length.

The PEG Unit provided herein comprises one or multiple polyethylene glycol chains. The polyethylene glycol chains can be linked together, for example, in a linear, branched or star shaped configuration. Typically, at least one of the polyethylene glycol chains of the PEG Unit is derivitized at one end for covalent attachment to an appropriate site on a component of the Multiplexer Linking Assembly Unit (e.g. A, M, T^{MC}, or D^{M}). Exemplary attachments to the Multiplexer Linking Assembly Unit are by means of non-conditionally cleavable linkages or via conditionally cleavable linkages. Exemplary attachments are via amide linkage, ether linkages, ester linkages, hydrazone linkages, oxime linkages, disulfide linkages, peptide linkages or triazole linkages. In some aspects, attachment to the Multiplexer Linking Assembly Unit is by means of a non-conditionally cleavable linkage. In some aspects, attachment to the Multiplexer Linking Assembly Unit is not via an ester linkage, hydrazone linkage, oxime linkage, or disulfide linkage. In some aspects, attachment to the Multiplexer Linking Assembly Unit is not via a hydrazone linkage.

A conditionally cleavable linkage refers to a linkage that is not substantially sensitive to cleavage while circulating in plasma but is sensitive to cleavage in an intracellular or intratumoral environment. A non-conditionally cleavable linkage is one that is not substantially sensitive to cleavage in any biological environment in a subject that is administered the TM-ADC. Chemical hydrolysis of a hydrazone, reduction of a disulfide, and enzymatic cleavage of a peptide bond or glycosidic linkage are examples of conditionally cleavable linkages.

The PEG Unit will be directly attached to the TM-ADC (or Intermediate thereof) at the Multiplexer Linking Assembly Unit. The other terminus (or termini) of the PEG Unit will be free and untethered (i.e., not covalently attached) and may take the form of a methoxy, carboxylic acid, alcohol or other suitable functional group. The methoxy, carboxylic acid, alcohol or other suitable functional group acts as a cap for the terminal polyethylene glycol subunit of the PEG Unit. By untethered, it is meant that the PEG Unit will not be covalently attached at that untethered site to a Drug Moiety, to an antibody, or to a linking component to a Drug Unit and/or an antibody. Such an arrangement will allow a PEG Unit of sufficient length to assume a parallel orientation with respect to a hydrophobic Drug Moiety (D^{M}) or Drug Unit (D^{U}) so as to mask its hydrophobicity, as discussed in more detail herein, thus allowing in such instances for the higher loading provided by the MLA Unit. For those embodiments in which the PEG Unit comprises more than one polyethylene glycol chain, the multiple polyethylene glycol chains may be independently chosen, e.g., be the same or different chemical moieties (e.g., polyethylene glycol chains of different molecular weight or number of subunits). A PEG Unit having multiple polyethylene glycol chains is attached to the Multiplexer Linking Assembly Unit at a single attachment site. The skilled artisan will understand that the PEG Unit in addition to comprising repeating polyethylene glycol subunits may also contain non-PEG material (e.g., to facilitate coupling of multiple polyethylene glycol chains to each other or to facilitate coupling to the Multiplexer Linking Assembly Unit). Non-PEG material refers to the atoms in the PEG Unit that are not part of the repeating —CH₂CH₂O— subunits. In some embodiments provided herein, the PEG Unit comprises two monomeric PE polyethylene glycol G chains attached to each other via non-PEG elements. In other embodiments provided herein, the PEG Unit comprises two linear polyethylene glycol chains attached to a central core that is attached to the Multiplexer Linking Assembly Unit (i.e., the PEG Unit itself is branched).

In some embodiments, the PEG Unit is divalent linking component. That is, both termini are attached to a component of a Multiplexer Linking Assembly Unit. In some embodiments, the points of attachment of the PEG Unit are with the same component of the Multiplexer Linking Assembly (e.g. Linking Group (A)). In some embodiments, the points of attachment of the PEG Unit are to two different components of the Multiplexer Linking Assembly Unit (e.g. Linking Group (A) and Multiplexer Group (M). In some embodiments, the PEG Unit is a divalent linking component of the Linking Group.

There are a number of PEG attachment methods available to those skilled in the art, [see, e.g., Goodson, et al. (1990) Bio Technology 8:343 (PEGylation of interleukin-2 at its glycosylation site after site-directed mutagenesis); EP 0 401 384 (coupling PEG to G-CSF); Malik, et al., (1992) Exp. Hematol. 20:1028-1035 (PEGylation of GM-CSF using tresyl chloride); ACT Pub. No. WO 90/12874 (PEGylation of erythropoietin containing a recombinantly introduced cysteine residue using a cysteine-specific mPEG derivative); U.S. Pat. No. 5,757,078 (PEGylation of EPO peptides); U.S. Pat. No. 5,672,662 (Poly(ethylene glycol) and related polymers monosubstituted with propionic or butanoic acids and functional derivatives thereof for biotechnical applications); U.S. Pat. No. 6,077,939 (PEGylation of an N-terminal .alpha.-carbon of a peptide); Veronese et al., (1985) Appl. Biochem. Bioechnol 11:141-142 (PEGylation of an N-terminal α-carbon of a peptide with PEG-nitrophenylcarbonate (“PEG-NPC”) or PEG-trichlorophenylcarbonate); and Veronese (2001) Biomaterials 22:405-417 (Review article on peptide and protein PEGylation)].

For example, a PEG Unit may be covalently bound to an amino acid residue via reactive groups of a polyethylene glycol-containing compound and the amino acid residue. Reactive groups of the amino acid residue include those that are reactive to an activated PEG molecule (e.g., a free amino or carboxyl group). For example, N-terminal amino acid residues and lysine (K) residues have a free amino group; and C-terminal amino acid residues have a free carboxyl group. Thiol groups (e.g., as found on cysteine residues) are also useful as a reactive group for forming a covalent attachment to a PEG Unit. In addition, enzyme-assisted methods for introducing activated groups (e.g., hydrazide, aldehyde, and aromatic-amino groups) specifically at the C-terminus of a polypeptide have been described (see Schwarz, et al. (1990) Methods Enzymol. 184:160; Rose, et al. (1991) Bioconjugate Chem. 2:154; and Gaertner, et al. (1994) J. Biol. Chem. 269:7224].

In some embodiments, a polyethylene glycol-containing compound forms a covalent attachment to an amino group using methoxylated PEG (“mPEG”) having different reactive moieties. Non-limiting examples of such reactive moieties include succinimidyl succinate (SS), succinimidyl carbonate (SC), mPEG-imidate, para-nitrophenylcarbonate (NPC), succinimidyl propionate (SPA), and cyanuric chloride. Non-limiting examples of such mPEGs include mPEG-succinimidyl succinate (mPEG-SS), mPEG₂-succinimidyl succinate (mPEG₂-SS); mPEG-succinimidyl carbonate (mPEG-SC), mPEG₂-succinimidyl carbonate (mPEG₂-SC); mPEG-imidate, mPEG-para-nitrophenylcarbonate (mPEG-NPC), mPEG-imidate; mPEG₂-para-nitrophenylcarbonate (mPEG₂-NPC); mPEG-succinimidyl propionate (mPEG-SPA); mPEG₂-succinimidyl propionate (mPEG, -SPA); mPEG-N-hydroxy-succinimide (mPEG-NHS); mPEG₂-N-hydroxy-succinimide (mPEG₂-NHS); mPEG-cyanuric chloride; mPEG₂-cyanuric chloride; mPEG₂-Lysinol-NPC, and mPEG₂-Lys-NHS.

Generally, at least one of the polyethylene glycol chains that make up the PEG Unit is functionalized to provide covalent attachment to the Multiplexer Linking Assembly Unit. Functionalization of the polyethylene glycol-containing compound that is the precursor to the PEG Unit includes, for example, via an amine, thiol, NHS ester, maleimide, alkyne, azide, carbonyl, or other functional group. In some embodiments, the PEG Unit further comprises non-PEG material (i.e., material not comprised of —CH₂CH₂O—) that provides coupling to the Multiplexer Linking Assembly Unit or in constructing the polyethylene glycol-containing compound or PEG Unit facilitates coupling of two or more polyethylene glycol chains.

The presence of the PEG Unit in a Multiplexer Linking Assembly Unit is capable of having two potential impacts upon the pharmacokinetics of the resulting TM-ADC. The desired impact is a decrease in clearance (and consequent increase in exposure) that arises from the reduction in non-specific interactions induced by the exposed hydrophobic elements of the Drug Unit. The second impact is undesired and is a decrease in volume and rate of distribution that sometimes arises from the increase in the molecular weight of the TM-ADC. Increasing the number of polyethylene glycol subunits increases the hydrodynamic radius of a conjugate, typically resulting in decreased diffusivity. In turn, decreased diffusivity typically diminishes the ability of the TM-ADC to penetrate into a tumor (Schmidt and Wittrup, Mol Cancer Ther 2009; 8:2861-2871). Because of these two competing pharmacokinetic effects, it is desirable to use a PEG that is sufficiently large to decrease the TM-ADC clearance thus increasing plasma exposure, but not so large as to greatly diminish its diffusivity to an extent that it interferes with the ability of the TM-ADC to reach the intended target cell population (e.g., see examples 1, 18, and 21 of US2016/0310612, which is incorporated by reference herein, for methodology for selecting an optimal PEG size for a particularly drug-linker moiety).

In one group of embodiments, the PEG Unit comprises one or more linear polyethylene glycol chains each having at least 2 subunits, at least 3 subunits, at least 4 subunits, at least 5 subunits, at least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In preferred embodiments, the PEG Unit comprises a combined total of at least 6 subunits, at least 8, at least 10 subunits, or at least 12 subunits. In some such embodiments, the PEG Unit comprises no more than a combined total of about 72 subunits, preferably no more than a combined total of about 36 subunits. In some embodiments, the PEG Unit comprises between about 2 and about 12 subunits.

In another group of embodiments, the PEG Unit comprises a combined total of from 4 to 72, 4 to 60, 4 to 48, 4 to 36 or 4 to 24 subunits, from 5 to 72, 5 to 60, 5 to 48, 5 to 36 or 5 to 24 subunits, from 6 to 72, 6 to 60, 6 to 48, 6 to 36 or from 6 to 24 subunits, from 7 to 72, 7 to 60, 7 to 48, 7 to 36 or 7 to 24 subunits, from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

Illustrative linear PEG Units that can be used in any of the embodiments provided herein are as follows:

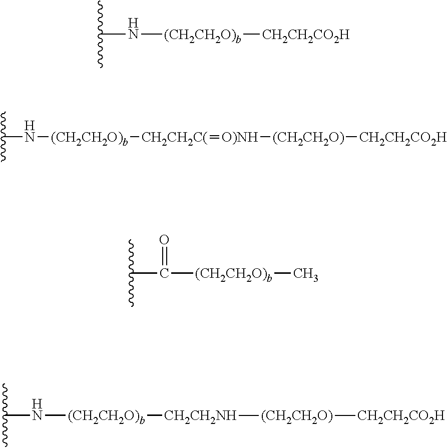

wherein the wavy line indicates site of attachment to the Multiplexer Linking Assembly, and each n is independently selected from 4 to 72, 6 to 72, 8 to 72, 10 to 72, 12 to 72, 6 to 24, or 8 to 24. In some embodiments, subscript b is about 8, about 12, or about 24.

As described herein, the PEG Unit is selected such that it improves clearance of the resultant TM-ADC but does not significantly impact the ability of the Conjugate to penetrate into the tumor. In embodiments in which the Drug Moiety and Multiplexer Linking Assembly Unit of the TM-ADC has a hydrophobicity comparable to that of a maleimido-derived glucuronide MMAE Drug Moiety, the PEG Unit to be selected for use will preferably have from 8 subunits to about 24 subunits, more preferably about 12 subunits. In embodiments in which the Drug Moiety and Multiplexer Linking Assembly Unit of the TM-ADC has a hydrophobicity greater than that of a maleimido-derived glucuronide MMAE Drug Moiety, a PEG unit with more subunits is sometimes required.

In preferred embodiments of the present disclosure the PEG Unit is from about 300 daltons to about 5 kilodaltons; from about 300 daltons to about 4 kilodaltons; from about 300 daltons to about 3 kilodaltons; from about 300 daltons to about 2 kilodaltons; from about 300 daltons to about 1 kilodalton; or any value in between. In some such aspects, the PEG Unit has at least 8, 10 or 12 subunits. In some such aspects, the PEG Unit has at least 8, 10 or 12 subunits but no more than 72 subunits, preferably no more than 36 subunits.

In preferred embodiments of the present disclosure, apart from the PEG Unit, there are no other PEG subunits present in the Multiplexer Linking Assembly (i.e., no PEG subunits in any of the other components of the conjugates and linkers provided herein). In other aspects of the present invention, apart from the PEG Unit, there are no more than 8, no more than 7, no more than 6, no more than 5, no more than 4, no more than 3, no more than 2 or no more than 1 other polyethylene glycol subunits present in the Multiplexer Linking Assembly (i.e., no more than 8, 7, 6, 5, 4, 3, 2, or 1 other polyethylene glycol subunits in other components of the TM-ADCs provided herein).

It will be appreciated that when referring to polyethylene glycol subunits of a PEG Unit, and depending on context, the number of subunits can represent an average number, e.g., when referring to a population of TM-ADCs or Intermediate Compounds thereto and/or using polydisperse PEGs.

### Additional Multiplexer Linking Assembly Embodiments

In addition to the Multiplexer Linking Assembly (MLA) embodiments discussed above (Formulas I, Ib, and Ic), the present disclosure provides additional MLA embodiments as described below. Thiol Multiplexers (T^{MC1}, T^{MC2}), Linking Groups (A¹ and A²), Partitioning Groups (Y), and Drug Moieties (D^{M}) in the formulas below have the same meaning as discussed in the sections above.

### Multiplexer (M) Group

A Multiplexer (M) Group in the MLA compounds and TM-ADCs described herein serves as a branching component (or trifunctional linking group). The initial Multiplexer (M) Group provides both covalent attachment to Linking Group (A′) as well as covalent attachments to two (A²-T^{MC2}) groups. Covalent attachments to Linking Group (A′) and two (A²-T^{MC2}) groups is achieved with from 1 to 3 functional groups. For example, in some embodiments the Multiplexer (M) Group is comprised of a single functional group, such as a single tertiary amine, providing covalent attachment to the Linking Group (A¹) as well as covalent attachment to two (A²-T^{MC2}) groups. Alternatively, in some embodiments, the Multiplexer (M) Group is comprised of two or three functional groups that provides covalent attachments to a Linking Group (A¹) and two (A²-T^{MC2}) groups. For example, in some embodiments, a thiol, a hydroxyl, an amine or other nucleophilic group provide covalent attachment to the Linking Group (A¹), while a covalent attachment to either or both of the (A²-T^{MC2}) groups is provided by a thiol, a hydroxy, an amine, or another nucleophilic group. In embodiments where the Multiplexer (M) Group is comprised of two or more functional groups, the two or more functional groups are linked by a variety of suitable groups such as branched or unbranched C₁₋₈ alkylene moieties.

In some embodiments, a Multiplexer (M) Group is represented by a moiety having the Formula:

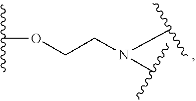

wherein, the wavy lines to the right are (A²-T^{MC2}) moieties, and the wavy line to the left is an A¹ group.

In some embodiments, a Multiplexer (M) Group is represented by a moiety having the Formula:

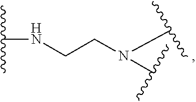

wherein, the wavy lines to the right are (A²-T^{MC2}) moieties, and the wavy line to the left is an A¹ group.

In some embodiments, a Multiplexer (M) Group is represented by a moiety having the Formula:

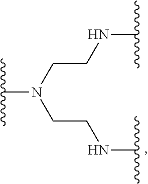

wherein, the wavy lines to the right are (A²-T^{MC2}) moieties, and the wavy line to the left is an A¹ group.

In some embodiments, a Multiplexer (M) Group is represented by a moiety having the Formula:

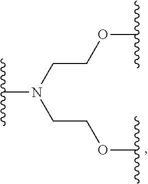

wherein, the wavy lines to the right are (A²-T^{MC2}) moieties, and the wavy line to the left is an A¹ group.

In some embodiments, a Multiplexer (M) Group is represented by a moiety having the Formula:

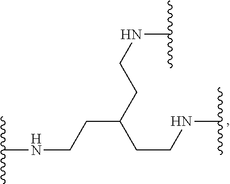

wherein, the wavy lines to the right are (A²-T^{MC2}) moieties, and the wavy line to the left is an A¹ group.

From the above description, it is also apparent that a Thiol Multiplexers (T^{MC}) Group is one type of Multiplexer (M).

The functional groups of a Multiplexers (M) Group described above are all nucleophilic groups; however, a person of skill in the art will recognize that the choice of nucleophilic group or electrophilic group for covalent attachment to A¹ or (A²-T^{MC2}) can be changed without departing from the scope of the current disclosure. It is apparent that the choice of nucleophilic group or electrophilic group depends on the chemical identity of the functional group providing covalent attachment to the Multiplexer (M) Group in the A¹ or (A²-T^{MC2}) groups.

Although the above description centers on Multiplexer (M) Groups providing covalent attachment to Linking Group (A′) as well as covalent attachment to two (A²-T^{MC2}) groups, Multiplexer (M) Groups are possible at any suitable branching position.

### Antibodies (Ab)

Antibodies useful in the TM-ADCs described herein are essentially any antibodies or fragments thereof targeting an antigen related to a clinically relevant disease state. This includes antibody fragments as well as antibodies having four available inter-chain disulfide linkages, or the eight thiols that are produced by reduction of those inter-chain disulfide linkages. The antibodies of the present disclosure can be non-engineered antibodies—antibodies in which no modifications are made to introduce additional amino acids or peptides, or engineered antibodies—antibodies in which one or more engineered cysteine residues are incorporated into an antibody or a fragment thereof.

In some embodiments, the antibodies of the present disclosure include one or more engineered cysteine (eCys) residues. An eCys residue is a cysteine amino acid or a derivative thereof that is incorporated into the heavy chain or light chain of an antibody, typically the one or more eCys residues are incorporated into the antibody by mutagenizing the parent antibody. Further information can be found in U.S. Pat. No. 9,000,130, the contents of which is incorporated herein for all purposes. In some embodiments, derivatives of cysteine (Cys) include, but are not limited to beta-2-Cys, beta-3-Cys, homocysteine, and N-methyl cysteine.

In some embodiments, the antibodies (Ab) are those that are intact or fully-reduced antibodies. The term ‘fully-reduced’ is meant to refer to antibodies in which all four inter-chain disulfide linkages have been reduced to provide eight thiols that can be attached to Linking Group A¹.

In some embodiments, the antibodies (Ab) are those that are intact or fully-reduced antibodies, or are antibodies bearing engineered cysteine groups that are modified with a functional group that can participate in, for example, Click chemistry or other cycloaddition reactions for attachment of MLA components as described herein.

In one group of embodiments, a Thiol Multiplexed Antibody Drug Conjugate (TM-ADC) compound is represented by Formula I^{Ab}:

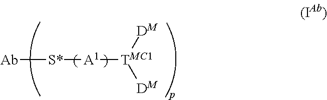

wherein:
Ab is from an antibody;
S* is a sulfur atom from a reduced interchain disulfide bond or a sulfur atom of the antibody from an engineered cysteine unit, or S* represents a modified functional group that was introduced into the antibody or moeity covalently attached to the antibody that resulted from Click Chemistry, Diel-Alder Chemistry or other cycloaddition reaction;
A¹ is a first Linking Group;
each A² is independently a bond or an independently selected second linking group;
each of A¹ and A² optionally comprises a Partitioning Group (Y) that is covalently attached to the first or second Linking Groups, or is a divalent linking component of the first or second Linking Group;
M is a Multiplexing Group or a first Thiol Multiplexing Group (T^{MC1});
each D^{M} is a Drug Moiety; and subscript p is an integer of from 1 to 10.

In some embodiments, provided herein is a Thiol Multiplexed Antibody Drug Conjugate compound is represented by formula I^{Ab}a:

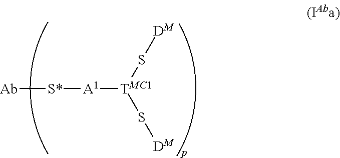

wherein:
Ab is from an antibody;
S* is a sulfur atom from a reduced interchain disulfide bond or a sulfur atom from an engineered cysteine unit of the antibody, or S* represents a modified functional group that was introduced into the antibody or moeity covalently attached to the antibody that resulted from Click Chemistry, Diel-Alder Chemistry or other cycloaddition reaction;
A¹ is a first Linking Group, optionally comprising a Partitioning Group (Y) that is covalently attached to the first Linking Group, or is a divalent linking component of the first Linking Group;
T^{MC1} is a Thiol Multiplexing Group;
each S is a sulfur atom from the Thiol Multiplexing Groups (T^{MC1});
each D^{M} is a Drug Moiety; and subscript p is an integer of from 1 to 10.

In one group of embodiments, a Thiol Multiplexed Antibody Drug Conjugate (TM-ADC) compound is represented by Formula II^{Ab}:

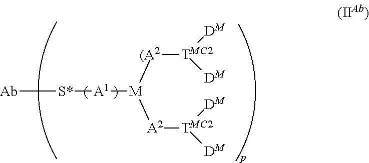

wherein:
Ab is from an antibody;
S* is a sulfur atom from a reduced interchain disulfide bond or a sulfur atom from an engineered cysteine unit of the antibody, or S* represents a modified functional group that was introduced into the antibody or moeity covalently attached to the antibody that resulted from Click Chemistry, Diel-Alder Chemistry or other cycloaddition reaction;
A¹ is a first Linking Group;
each A² is independently a bond or an independently selected second linking group;
each of A¹ and A² optionally comprises a Partitioning Group (Y) that is covalently attached to the first or second Linking Groups, or is a divalent linking component of the first or second Linking Group;
M is a Multiplexing Group or a first Thiol Multiplexing Group (T^{MC1});
each T^{MC2} is a second Thiol Multiplexing Group in reduced form;
each D^{M} is a Drug Moiety attached to a sulfur atom from a T^{MC2} Group in reduced form; and subscript p is an integer of from 1 to 10

In one group of embodiments, the thiol multiplex antibody drug conjugates (TM-ADC) are represented by Formula I^{Ab}b:

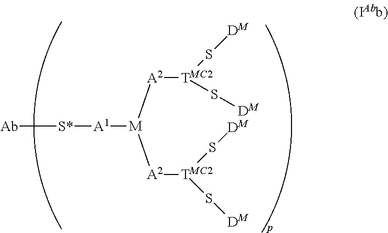

wherein:
Ab is from an antibody;
S* is a sulfur atom from a reduced interchain disulfide bond or a sulfur atom from an engineered cysteine unit of the antibody, or S* represents a modified functional group that was introduced into the antibody or moeity covalently attached to the antibody that resulted from Click Chemistry, Diel-Alder Chemistry or other cycloaddition reaction;
A¹ is a first Linking Group;
each A² is independently a bond or an independently selected second linking group;
each of A¹ and A² comprises 0 or 1 Partitioning Group (Y) that is covalently attached to the first or second Linking Groups, or a divalent linking component of the Linking Group;
M is a Multiplexing Group or a first Thiol Multiplexing Group (T^{MC1});
each T^{MC2} is independently a second Thiol Multiplexing Group;
each S is a sulfur atom of the second Thiol Multiplexing Group (T^{MC2}); and
each D^{M} is a Drug Moiety.

In one group of embodiments, the antibody for any of the TM-ADCs described herein is directed against a cancer cell antigen. In another group of embodiments, the antibody is directed against a bacteria-related antigen. In yet another group of embodiments, the antibody is directed against an autoimmune cell antigen. It will be understood that the antibody component in a TM-ADC is an antibody in residue form such that Ab in the TM-ADC structures described herein incorporates the structure of the antibody.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The term “antibody” in some aspects further includes a functionally active fragment, derivative or analog of an antibody that immunospecifically binds to target cells (e.g., cancer cell antigens, viral antigens, or microbial antigens) or other antibodies bound to tumor cells or matrix. In this regard, “functionally active” means that the fragment, derivative or analog is able to immunospecifically binds to target cells. To determine which CDR sequences bind the antigen, synthetic peptides containing the CDR sequences are typically used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md.; Kabat E et al., 1980, J. Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060; each of which is incorporated herein by reference in its entirety.

Completely human antibodies are particularly desirable in some embodiments and are typically produced using transgenic mice that are incapable of expressing endogenous immunoglobulin heavy and light chains genes, but which are capable of expressing human heavy and light chain genes.

Antibodies immunospecific for a cancer cell antigen are obtainable commercially or produced by any method known to one of skill in the art such as, e.g., chemical synthesis or recombinant expression techniques. The nucleotide sequence encoding antibodies immunospecific for a cancer cell antigen are obtainable, e.g., from the GenBank database or a database like it, the literature publications, or by routine cloning and sequencing.

In a specific embodiment, known antibodies for the treatment of cancer are used. Antibodies immunospecific for a cancer cell antigen are obtainable commercially or produced by any method known to one of skill in the art such as, e.g., recombinant expression techniques. The nucleotide sequence encoding antibodies immunospecific for a cancer cell antigen are obtainable, e.g., from the GenBank database or a database like it, the literature publications, or by routine cloning and sequencing.

In another specific embodiment, antibodies for the treatment of an autoimmune disease are used in accordance with the compositions and methods of the invention. Antibodies immunospecific for an antigen of a cell that is responsible for producing autoimmune antibodies are obtainable if not commercially or otherwise available by any method known to one of skill in the art such as, e.g., chemical synthesis or recombinant expression techniques.

In certain embodiments, useful antibodies are to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein.

Exemplary attachment to the antibody is via thioether linkages.

### Cancer Cell Antigens

Examples of antibodies available for the treatment of cancer to and internalizing antibodies that bind to tumor associated antigens are reviewed in Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998.

### Select Embodiments of Linking Assembly Units and TM-ADCs

In some embodiments, A¹ of the MLA represented by Formulas I, II, III comprises a maleimido group.

The MLA represented by Formulas I and II, wherein A¹ is a self-stabilizing moiety.

In some selected embodiments, the MLA compounds of Formula (I) and (II) are those in which A¹ has a formula selected from the group consisting of:

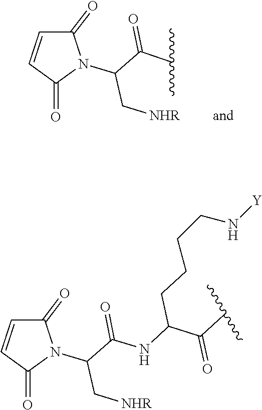

wherein R is selected from the group consisting of H and an amine protecting group; Y is a Partitioning Group; and the wavy line indicates attachment to T^{MC1}.

In some selected embodiments, the MLA compounds of Formula (I) are those in which T^{MC1} is in disulfide form and is selected from the group consisting of:

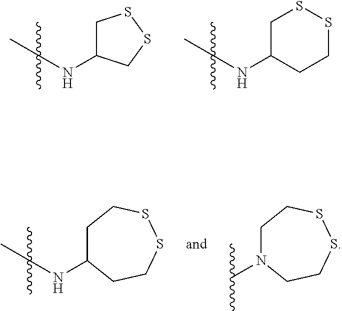 [SMILES: *NC1CSSC1 ; *NC1CCSSC1 ; *NC1CCSSCC1 ; *N1CCSSCC1]

In some selected embodiments, the MLA compounds of Formula (I) are those in which subscript m is 0, M is T^{MC1}, and T^{MC1} is selected from the group consisting of:

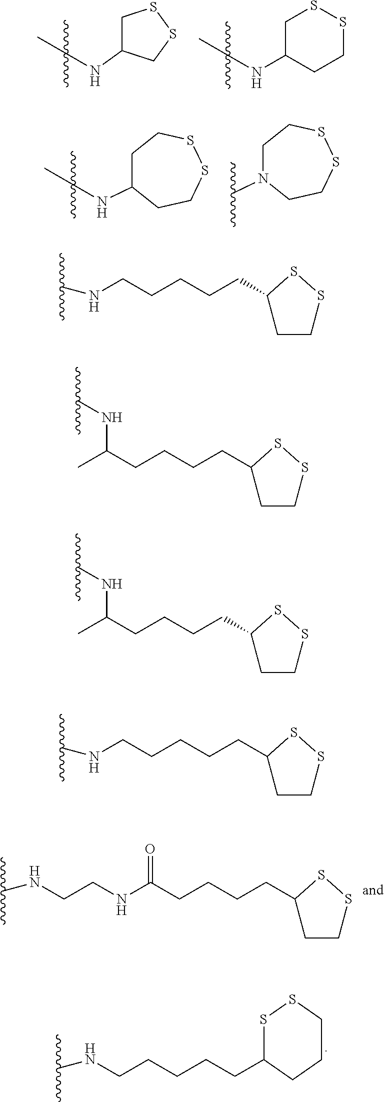

In some selected embodiments, the MLA compounds of Formula (I) are those in which subscript m is 0 and T^{MC1} is selected from the group consisting of:

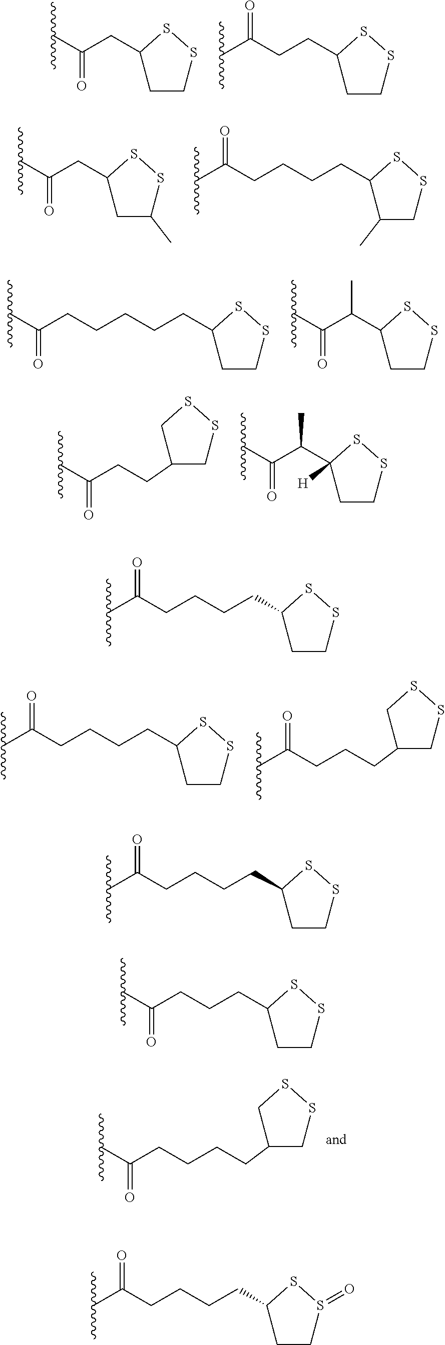

In some selected embodiments, the MLA compounds of formula (II) are those in which subscript m is 1, T^{MC} a is selected from the group consisting of:

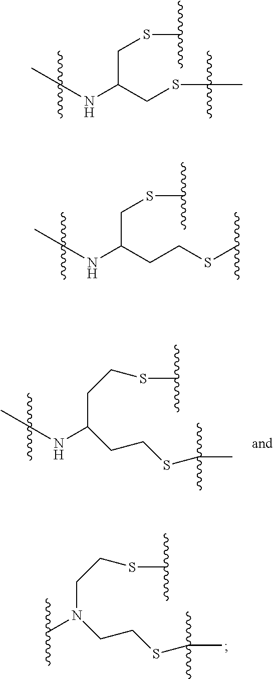 [SMILES: *NC(CS*)CS* ; *NC(CCS*)CS* ; *NC(CCS*)CCS* ; *SCCN(*)CCS*]

T^{MC2} is in disulfide form and is selected from the group consisting of:

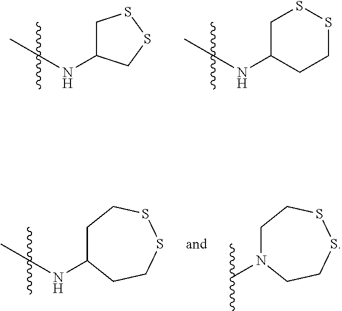 [SMILES: *NC1CSSC1 ; *NC1CCSSC1 ; *NC1CCSSCC1 ; *N1CCSSCC1]

In some selected embodiments, the MLA compounds of formula (II) are those in which T^{MC1} and T^{MC2} are each independently selected from the group consisting of:

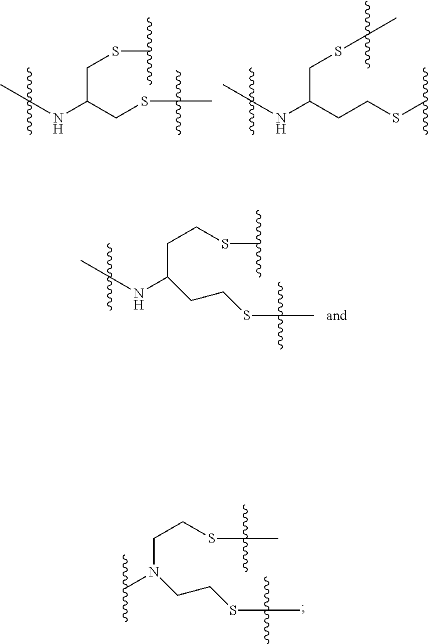 [SMILES: *NC(CS*)CS* ; *NC(CCS*)CS* ; *NC(CCS*)CCS* ; *SCCN(*)CCS*]

and each T^{MC2} is also attached to two Drug Moieties.

In some selected embodiments, the MLA compounds of formula (II) are those in which each of (A²-T^{MC2}) has a formula independently selected from the group consisting of:

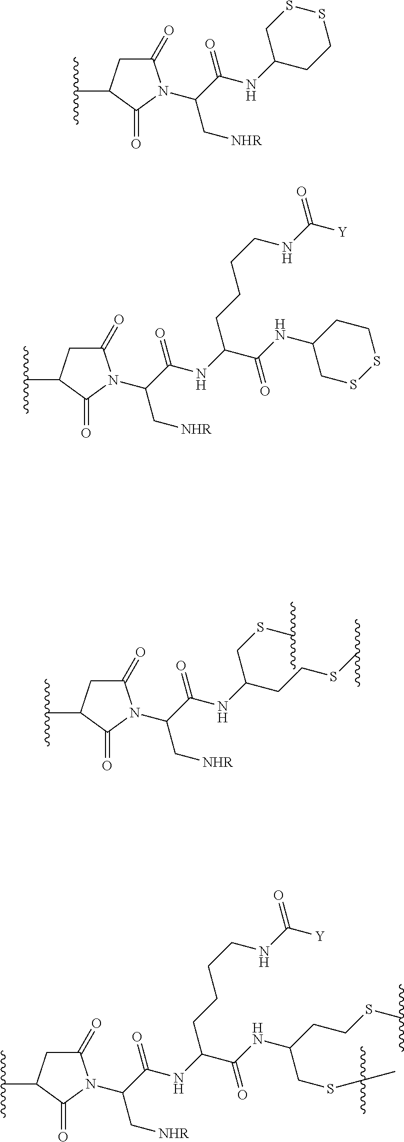

wherein the wavy line to the left of the succinimide ring indicates thioether attachment to T^{MC1} and each of the wavy lines to the right of the sulfur atoms indicates covalent attachment to a Drug Moiety (D^{M}).

In some embodiments, the MLA compounds of Formula (I) or (II) are those in which Drug Moieties are attached, each of said Drug Moieties having a formula selected from the group consisting of mc-VC-PAB-D^{U}, me-D^{U}, mc-VC-D^{U}, MDpr-D^{U}, MDpr-Lys(PEG)-D^{U} and D^{U}; wherein me is the maleimide-derived succinimide moiety, optionally in hydrolyzed form, and each D^{U} is a Drug Unit incorporates the structure of a free drug selected from the group consisting of nucleoside chemotherapeutics; HDAC inhibitors; anthracyclines; NAMPT inhibitors; hydrophilic prodrugs (e.g., SN-38 glucuronide, etoposide phosphate); low molecular weight drugs (e.g., drugs having a molecular weight less than about 600); nitrogen mustards (e.g., melphalan); and proteosome inhibitors (e.g., lenolidomide). In some embodiments, D^{U} incorporates the structure of a free drug selected from the group consisting of nucleoside chemotherapeutics and antimetabolites such as antifolates (e.g., Methotrexate, Pemetrexed, L-leucovorin, GW1843, Raltitrexed, ZD9331, Pralatrexate, Lometrexol).

In some embodiments, the MLA compounds of Formula (I) are those having a formula selected from the group consisting of:

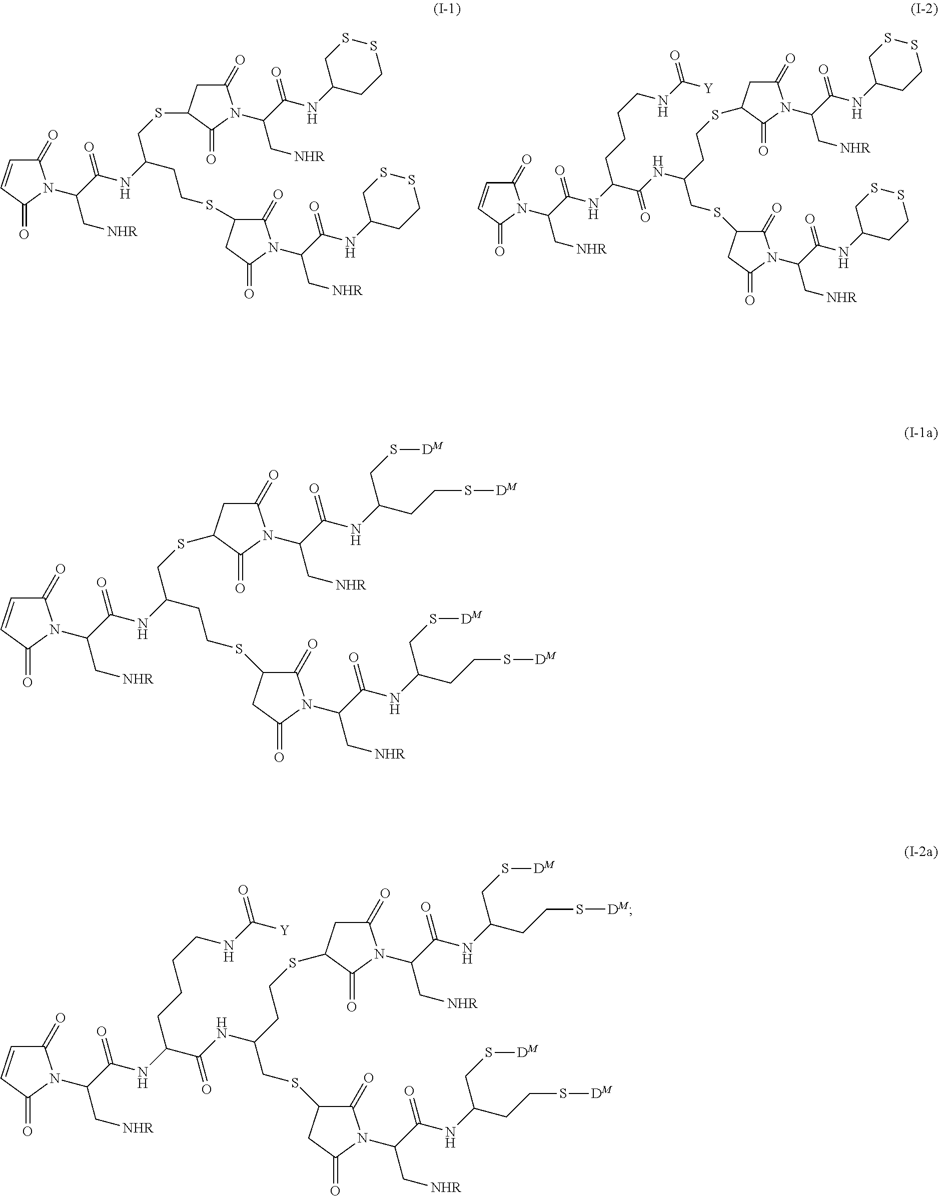

wherein each R is H or an amine protecting group; each D^{M} is a Drug Moiety; and Y is a Partitioning Group. In some embodiments, the Partitioning Group (Y) is a PEG Unit.

In some selected embodiments, the MLA compounds of formulae (I-1a) or (I-2a) are those in which D^{M} is a Drug Moiety having a formula selected from the group consisting of mc-VC-PAB-D^{U}, me-D^{U}, mc-VC-D^{U}, MDpr-D^{U}, MDpr-Lys(PEG)-D^{U} and D^{U}; wherein me is the maleimide-derived succinimide moiety, optionally in hydrolyzed form, and each D^{U} is a Drug Unit incorporates the structure of a free drug selected from the group consisting nucleoside chemotherapeutics; HDAC inhibitors; anthracyclines; NAMPT inhibitors; hydrophilic prodrugs (e.g., SN-38 glucuronide, etoposide phosphate); low molecular weight drugs (e.g., drugs having a molecular weight less than about 600); nitrogen mustards (e.g., melphalan); and proteosome inhibitors (e.g., lenolidomide). In some embodiments, D^{U} incorporates the structure of a free drug selected from the group consisting of a nucleoside chemotherapeutic and an anti-metabolites such as antifolates (e.g., Methotrexate, Pemetrexed, L-leucovorin, GW1843, Raltitrexid, ZD9331, Pralatrexate, Lometrexol).

In some selected embodiments each Drug Linker (D^{L}) in a Multiplexer Linking Assembly compound represented by Formulae I, II, and III, is a MDPr-vc linker.

In some selected embodiments a MLA compound represented by Formulas I, II, and III each Drug Linker (D^{L}) is independently selected from the group consisting of maleimido-caproyl (mc), maleimido-caproyl-valine-citrulline (mc-vc), and maleimido-caproyl-valine-citrulline-paraaminobenzyloxycarbonyl (mc-vc-PABC).

For ease of reference to the compounds and assemblies described herein, the component mc-vc-PAB-D^{U} in which me is a maleimide-derived succinimide moiety has the structure:

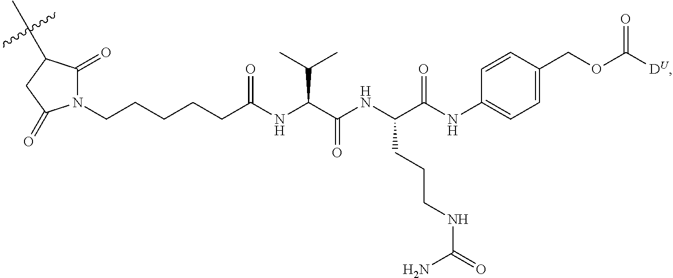

the component mc-VA-PAB-D^{U} in which me is a maleimide-derived succinimide moiety has the structure:

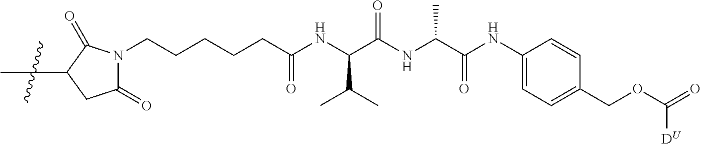

the component me-VA-D^{U} in which me is a maleimide-derived succinimide moiety has the structure:

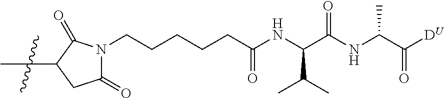

and the component MDpr-PAB(gluc)-D^{U} in which MDpr is a maleimide-derived succinimide moiety has the structure:

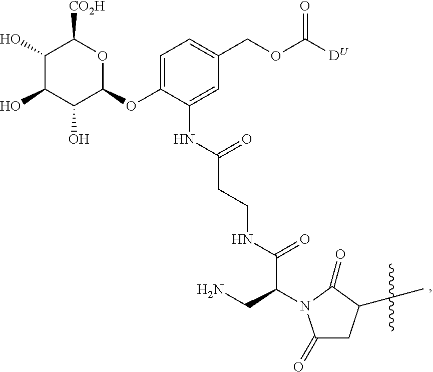

wherein mc-VC-PAB-D^{U}, mc-VA-PAB-D^{U}, me-VA-D^{U}, and MDpr-PAB(gluc)-D^{U} in which me and MDPr are maleimide-derived succinimide moieties are exemplary —D^{M} moieties bonded to a Multiplexer Linking Assembly Unit, and wherein the wavy line indicates covalent bonding of the succinimide ring of me or MDpr to a thiol present on a Thiol Multiplexer (T^{MC}) Group.

In any one of the above maleimide-derived succinimide moieties, the succinimide ring is optionally in hydrolyzed form and for succinimide moieties derived from MDpr the succinimide ring is preferably in hydrolyzed form.

In some embodiments, the MLA compound of Formula (I) or (II), is one wherein A¹-T^{MC1} comprises

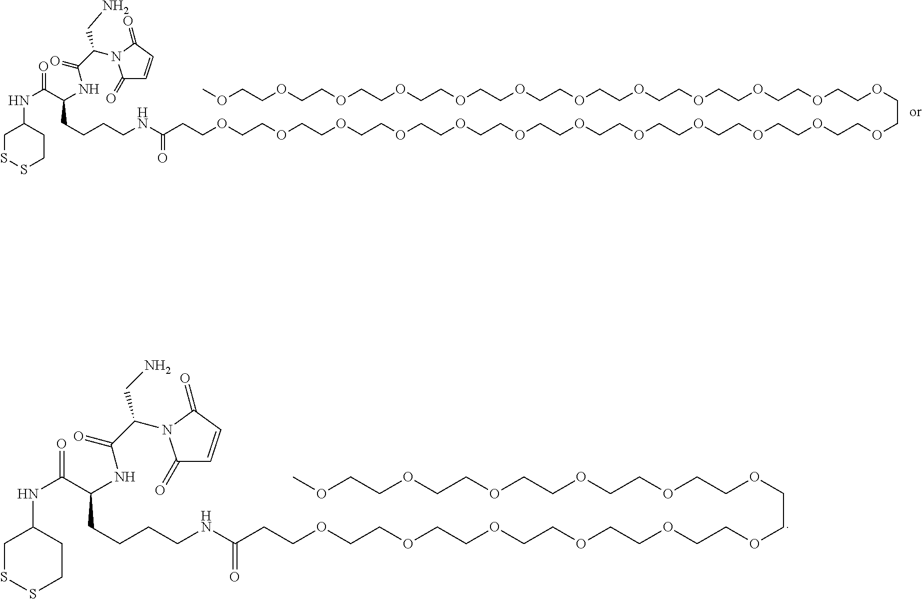 [SMILES: COCCOCCOCCOCCOCCOCCOCCOCCOCCOCCOCCOCCOCCOCCOCCOCCOCCOCCOCCOCCOCCOCCOCCOCCC(=O)NCCCC[C@H](NC(=O)[C@H](CN)N1C(=O)C=CC1=O)C(=O)NC1CCSSC1 ; COCCOCCOCCOCCOCCOCCOCCOCCOCCOCCOCCOCCC(=O)NCCCC[C@H](NC(=O)[C@H](CN)N1C(=O)C=CC1=O)C(=O)NC1CCSSC1]

In some embodiments, the MLA compound of Formula (I) or (II), is one wherein A¹-T^{MC1} comprises

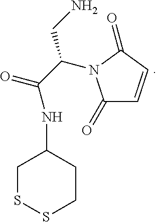 [SMILES: NC[C@@H](C(=O)NC1CCSSC1)N1C(=O)C=CC1=O]

In some embodiments, the MLA compound of Formula (I) or (II), is one wherein A¹-T^{MC1} comprises

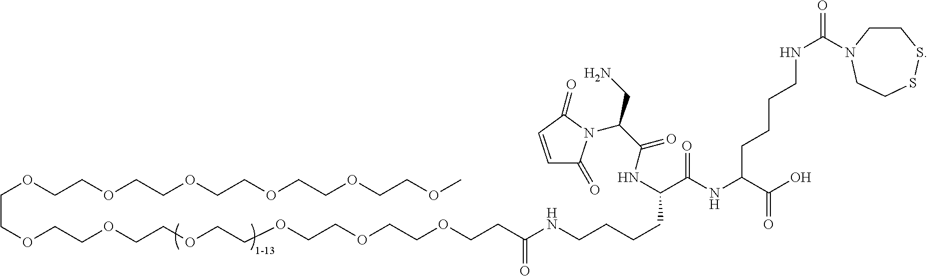

In some embodiments, the MLA compound of Formula (I) or (II), is one wherein A¹-T^{MC1} comprises

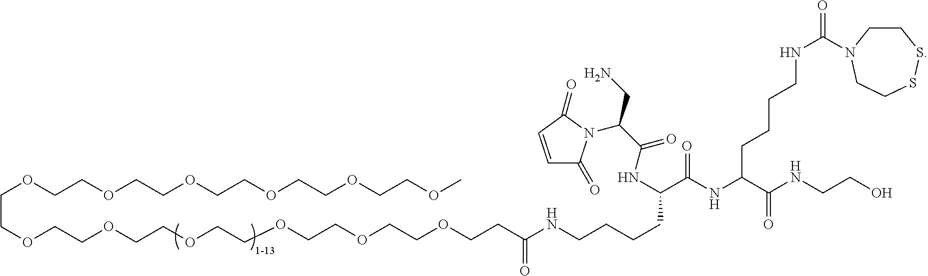

In some embodiments, the MLA compound of Formula (I) or (II), is one wherein A¹-T^{MC1} comprises

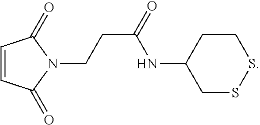 [SMILES: O=C(CCN1C(=O)C=CC1=O)NC1CCSSC1]

In some embodiments, the MLA compound of Formula (I) or (II), is one wherein A¹-T^{MC1} comprises

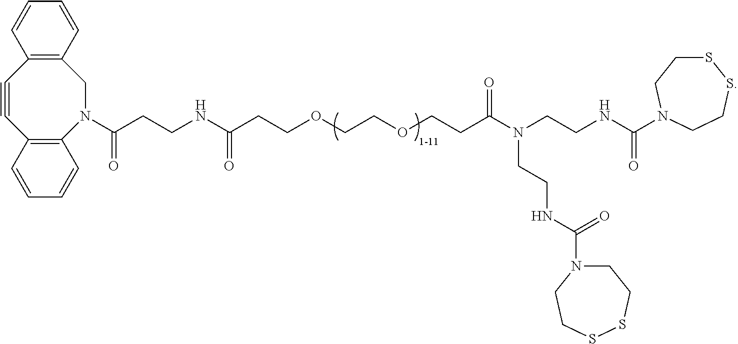

In some embodiments, the MLA compound of Formula (I) or (II), is one wherein A¹-T^{MC1} comprises

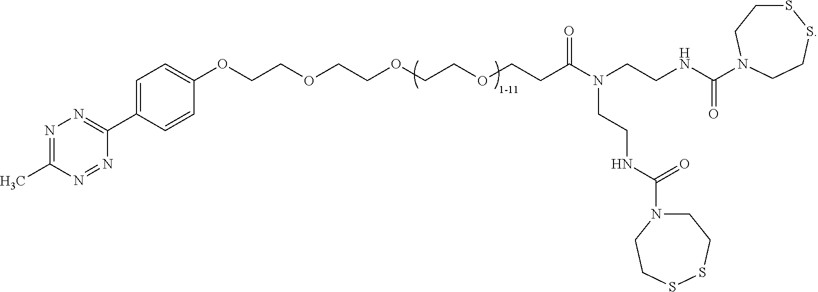

### Methods of Use

Treatment of Cancer

The TM-ADCs are useful for inhibiting the multiplication of a tumor cell or cancer cell, causing apoptosis in a tumor or cancer cell, or for treating cancer in a patient. The TM-ADCs can be used accordingly in a variety of settings for the treatment of cancers. The TM-ADCs can be used to deliver a drug to a tumor cell or cancer cell. Without being bound by theory, in one embodiment, the antibody of a TM-ADC binds to or associates with a cancer-cell or a tumor-cell-associated antigen, and the TM-ADC can be taken up (internalized) inside a tumor cell or cancer cell through receptor-mediated endocytosis or other internalization mechanism. The antigen can be attached to a tumor cell or cancer cell or can be an extracellular matrix protein associated with the tumor cell or cancer cell. Once inside the cell, via a cleavable mechanism, the drug is released within the cell. In an alternative embodiment, the Drug or Drug Unit is cleaved from the TM-ADC outside the tumor cell or cancer cell, and the Drug or Drug Unit subsequently penetrates the cell.

In one embodiment, the antibody binds to the tumor cell or cancer cell.

In another embodiment, the antibody binds to a tumor cell or cancer cell antigen which is on the surface of the tumor cell or cancer cell.

In another embodiment, the antibody binds to a tumor cell or cancer cell antigen which is an extracellular matrix protein associated with the tumor cell or cancer cell.

The specificity of the antibody for a particular tumor cell or cancer cell can be important for determining those tumors or cancers that are most effectively treated. For example, TM-ADCs that target a cancer cell antigen present on hematopoietic cancer cells in some embodiments treat hematologic malignancies. In other embodiments TM-ADCs that target a cancer cell antigen present on abnormal cells of solid tumors treat such solid tumors.

In other embodiments a TM-ADC are directed against abnormal cells of hematopoietic cancers such as, for example, lymphomas (Hodgkin Lymphoma and Non-Hodgkin Lymphomas) and leukemias and solid tumors.

Multi-Modality Therapy for Cancer

Cancers, including, but not limited to, a tumor, metastasis, or other disease or disorder characterized by abnormal cells that are characterized by uncontrolled cell growth in some embodiments are treated or inhibited by administration of a TM-ADC.

In other embodiments, methods for treating cancer are provided, including administering to a patient in need thereof an effective amount of a TM-ADC and a chemotherapeutic agent. In one embodiment the chemotherapeutic agent is that with which treatment of the cancer has not been found to be refractory. In another embodiment, the chemotherapeutic agent is that with which the treatment of cancer has been found to be refractory. In some of those embodiments the TM-ADCs is administered to a patient that has also undergone surgery as treatment for the cancer.

In some embodiments, the patient also receives an additional treatment, such as radiation therapy. In a specific embodiment, the TM-ADC is administered concurrently with the chemotherapeutic agent or with radiation therapy. In another specific embodiment, the chemotherapeutic agent or radiation therapy is administered prior or subsequent to administration of a TM-ADC.

In other embodiments, a chemotherapeutic agent is administered over a series of sessions. Any one or a combination of the chemotherapeutic agents, such a standard of care chemotherapeutic agent(s), can be administered.

Additionally, methods of treatment of cancer with a TM-ADC are provided as an alternative to chemotherapy or radiation therapy where the chemotherapy or the radiation therapy has proven or can prove too toxic, e.g., results in unacceptable or unbearable side effects, for the subject being treated. In some embodiments the patient being treated with a TM-ADC is also treated with another cancer treatment such as surgery, radiation therapy or chemotherapy, depending on which treatment is found to be acceptable or bearable.

Treatment of Autoimmune Diseases

The TM-ADCs are useful for killing or inhibiting the replication of a cell that produces an autoimmune disease or for treating an autoimmune disease. Thus, in some embodiments the TM-ADCs are used accordingly in a variety of settings for the treatment of an autoimmune disease in a patient. In other embodiments the TM-ADCs are used to deliver a drug to a target cell. Without being bound by theory, in one of those embodiments, a TM-ADC compound associates with an antigen on the surface of a target cell, and the TM-ADC compound is then taken up inside a target-cell through receptor-mediated endocytosis. Once inside the cell, the Linker unit is cleaved, resulting in release of the Drug or Drug Unit. The released Drug is then free to migrate in the cytosol and induce cytotoxic or cytostatic activities. In an alternative embodiment, the Drug is cleaved from the TM-ADC outside the target cell, and the Drug or Drug Unit subsequently penetrates the cell.

In one embodiment, the antibody binds to an autoimmune antigen. In one embodiment, the antigen is on the surface of a cell involved in an autoimmune condition.

In another embodiment, the antibody binds to an autoimmune antigen which is on the surface of a cell.

In one embodiment, the antibody binds to activated lymphocytes that are associated with the autoimmune disease state.

In a further embodiment, the TM-ADC kills or inhibit the multiplication of cells that produce an autoimmune antibody associated with a particular autoimmune disease.

Particular types of autoimmune diseases that can be treated with the TM-ADCs include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

### Multi-Drug Therapy of Autoimmune Diseases

Methods for treating an autoimmune disease are also disclosed including administering to a patient in need thereof an effective amount of a TM-ADC and another therapeutic agent known for the treatment of an autoimmune disease.

### Compositions and Methods of Administration

The present invention provides pharmaceutical compositions comprising the TM-ADCs described herein and a pharmaceutically acceptable carrier. The TM-ADCs are in any form that allows it to be administered to a patient for treatment of a disorder associated with expression of the antigen to which the antibody binds. For example, a TM-ADC will be in the form of a liquid or solid. The preferred route of administration is parenteral. Parenteral administration includes subcutaneous injections, intravenous, intramuscular, intrasternal injection or infusion techniques.

In one embodiment, the compositions are administered parenterally. In one of those embodiments, the conjugates are administered intravenously. Administration is typically through any convenient route, for example by infusion or bolus injection

Pharmaceutical compositions of a TM-ADC are formulated so as to allow a it to be bioavailable upon administration of the composition to a patient. Compositions will be in the form of one or more injectable dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

A TM-ADC composition is typically in the form of a liquid, suspension or lyophilized solid. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments the liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. Physiological saline is an exemplary adjuvant. An injectable composition is preferably a liquid composition that is sterile.

The amount of the TM-ADC that is effective in the treatment of a particular disorder or condition will depend on the nature of the disorder or condition, which is usually determined by standard clinical techniques. In addition, in vitro or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each patient's circumstances.

The compositions comprise an effective amount of a TM-ADC such that a suitable dosage will be obtained. Typically, this amount is at least about 0.01% of the TM-ADC by weight of the composition.

Generally, the dosage of a TM-ADC administered to a patient is typically from about 0.01 mg/kg to about 100 mg/kg, from about 1 to about 100 mg of a per kg or from about 0.1 to about 25 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is preferably between about 0.1 to 4 mg/kg, even more preferably 0.1 to 3.2 mg/kg, or even more preferably 0.1 to 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the conjugates are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions in some embodiments also include a Partitioning Group. Compositions for intravenous administration sometimes comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a TM-ADC is to be administered by infusion, it is sometimes dispensed, for example, with an infusion bottle containing sterile pharmaceutical grade water or saline. Where the conjugate is administered by injection, an ampoule of sterile water for injection or saline is typically provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

### EXAMPLES

Unless otherwise noted, all solvents and reagents were purchased from commercial sources in the highest purity possible and not further purified prior to use. Anhydrous solvents including dimethylformamide (DMF) and CH₂Cl₂ were purchased from Aldrich.

Products were purified by flash column chromatography utilizing a Biotage Isolera One flash purification system (Charlotte, N.C.). Preparative HPLC was carried out on a Waters 2454 Binary Gradient Module solvent delivery system configured with a Waters 2998 PDA detector. Products were purified with the appropriate diameter of column of a Phenomenex Max-RP 4 m Synergi 80 Å 250 mm reverse phase column eluting with 0.05% trifluoroacetic acid in water and 0.05% trifluoroacetic acid in acetonitrile unless otherwise specified. UPLC-MS was performed on a Waters single quad detector mass spectrometer interfaced to a Waters Acquity UPLC system using one of the following methods.

BEH C18 General Method: Acquity UPLC BEH C18 2.1×50 mm, 1.7 μm reversed-phase column; Solvent A—0.1% formic acid; Solvent B—acetonitrile with 0.1% formic acid

| Time (min) | Flow (mL/min) | A % | B % | Gradient |
|---|---|---|---|---|
| Initial | 0.5 | 97 | 3 | |
| 1.70 | 0.5 | 40 | 60 | Linear |
| 2.00 | 0.5 | 5 | 95 | Linear |
| 2.50 | 0.5 | 5 | 95 | Linear |
| 2.80 | 0.5 | 97 | 3 | Linear |
| 3.00 | 0.5 | 97 | 3 | Linear |

BEH C18 Hydrophobic Method: Acquity UPLC BEH C18 2.1×50 mm, 1.7 μm reversed-phase column; Solvent A—0.1% formic acid; Solvent B—acetonitrile with 0.1% formic acid

| Time (min) | Flow (mL/min) | A % | B % | Gradient |
|---|---|---|---|---|
| Initial | 0.5 | 97 | 3 | |
| 1.00 | 0.5 | 40 | 60 | Linear |
| 1.50 | 0.5 | 5 | 95 | Linear |
| 2.50 | 0.5 | 5 | 95 | Linear |
| 2.80 | 0.5 | 97 | 3 | Linear |
| 3.00 | 0.5 | 97 | 3 | Linear |

CORTECS C18 General Method: Waters CORTECS C18 1.6 μm, 2.1×50 mm, reversed-phase column; Solvent A—0.1% aqueous formic acid Solvent B—acetonitrile with 0.1% formic acid

| Time (min) | Flow (mL/min) | A % | B % | Gradient |
|---|---|---|---|---|
| Initial | 0.6 | 97 | 3 | |
| 1.70 | 0.6 | 40 | 60 | Linear |
| 2.00 | 0.6 | 5 | 95 | Linear |
| 2.50 | 0.6 | 5 | 95 | Linear |
| 2.80 | 0.6 | 97 | 3 | Linear |
| 3.00 | 0.6 | 97 | 3 | Linear |

CORTECS C18 Hydrophobic Method: Waters CORTECS C18 1.6 μm, 2.1×50 mm, reversed-phase column; Solvent A—0.1% aqueous formic acid; Solvent B—acetonitrile with 0.10% formic acid

| Time (min) | Flow (mL/min) | A % | B % | Gradient |
|---|---|---|---|---|
| Initial | 0.6 | 97 | 3 | |
| 1.50 | 0.6 | 5 | 95 | Linear |
| 2.40 | 0.6 | 5 | 95 | Linear |
| 2.50 | 0.6 | 97 | 3 | Linear |
| 2.80 | 0.6 | 97 | 3 | Linear |

CORTECS C18 Hydrophilic Method: Waters CORTECS C18 1.6 m, 2.1×50 mm, reversed-phase column; Solvent A—0.1% aqueous formic acid; Solvent B—acetonitrile with 0.1% formic acid

| Time (min) | Flow (mL/min) | A % | B % | Gradient |
|---|---|---|---|---|
| Initial | 0.6 | 97 | 3 | |
| 1.70 | 0.6 | 67 | 33 | Linear |
| 2.00 | 0.6 | 5 | 95 | Linear |
| 2.50 | 0.6 | 97 | 3 | Linear |
| 2.80 | 0.6 | 97 | 3 | Linear |

### Example 1

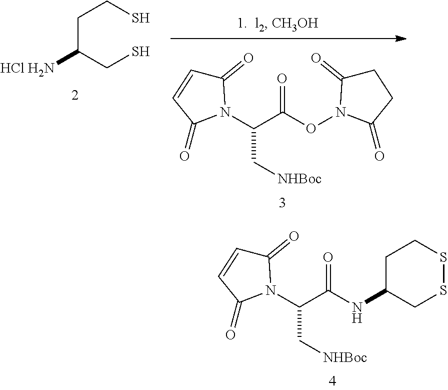

tert-butyl ((S)-3-(((S)-1,2-dithian-4-yl)amino)-2-(2,5-dioxo-2,5-dihydro-1H-pyrrol-1-yl)-3-oxopropyl)carbamate: Iodine (10 mg/mL) was added to a methanol solution of the dithiobutylamine HCl salt (2, 25 mg, 0.144 mmol) until the violet color of the solution persisted, indicating compete oxidation of the disulfide. After several hours, the mixture was concentrated. The residue was dissolved in DMF (0.5 mL) and Boc-DPR-OSu (3, 55 mg, 0.144 mmol) was added and the mixture was treated with DIPEA (100 μL). The reaction mixture was stirred 1 h and was concentrated. Ethyl acetate was added, and insoluble materials were removed by filtration. The resulting solution was chromatographed on a 1 mm radial chromatography plate eluting with 5 to 10% methanol/dichloromethane. Product-containing fractions were further purified on a 1 mm radial chromatography plate eluting with 50% ethyl acetate in hexanes, followed by 100% ethyl acetate, to give 12 mg (21%) of the title compound (4). Analytical UPLC-MS (BEH C18 General Method): tᵣ=1.85 min. MS (ESI+): 402.

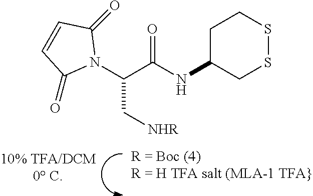

(S)-3-amino-2-(2,5-dioxo-2,5-dihydro-1H-pyrrol-1-yl)-N-((S)-1,2-dithian-4-yl)propanamide, trifluoroacetic acid salt: To a solution of the Boc-protected amine (4, 12 mg) in DCM (4.5 mL) at 0° C. was added trifluoroacetic acid (0.5 mL). The mixture was stirred for 2 h, and UPLC-MS analysis showed conversion to the desired amine. The reaction mixture was concentrated under reduced pressure. Dry DCM was added, and the mixture was concentrated to a residue a second time. The material was then taken up in dry dichloromethane concentrated under a stream of N₂ (2×) followed by high vacuum to give 16.5 mg of a white solid as the trifluoroacetic acid salt of the title compound (MLA-1 TFA). MLA-1 is an exemplary Multiplexer Linker Assembly compound of Formula Ia. Analytical UPLC-MS (BEH C18 General Method): tᵣ=0.92 min. MS (ESI+): 302.

### Example 2

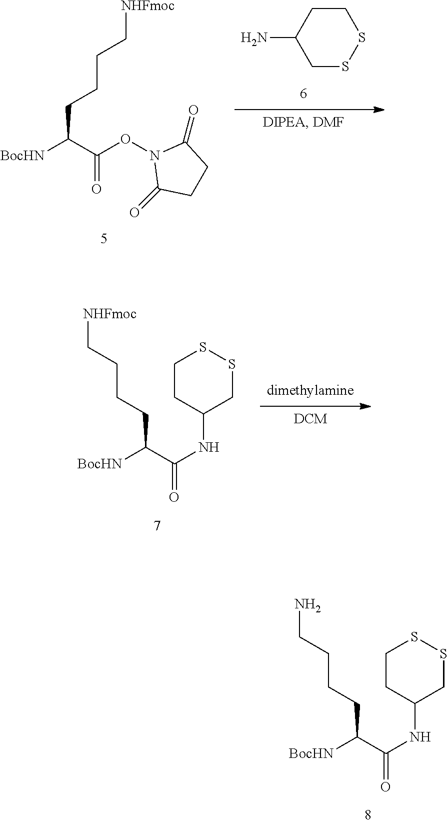

tert-butyl ((2S)-1-((1,2-dithian-4-yl)amino)-6-amino-1-oxohexan-2-yl)carbamate: 2,5-dioxopyrrolidin-1-yl N⁶-(((9H-fluoren-9-yl)methoxy)carbonyl)-N²-(tert-butoxycarbonyl)-L-lysinate (5, 1.03 g, 1.82 mmol), 1,2-dithian-4-amine (6, 235 mg, 1.74 mmol), prepared according to the procedures of Lyon, R. P. et al. Nature Biotechnol. (2014), 32(10) 1059-1065 and DIPEA (0.61 mL, 3.48 mmol) were mixed in anhydrous DMF (4 mL). The reaction mixture was stirred at room temp and monitored by LCMS. LCMS indicated full conversion of the disulfide after 2 hours. Solvents were removed by vacuum and the crude product was purified by silica gel chromatography (5% MeOH in DCM) to provide the disulfide intermediate (9H-fluoren-9-yl)methyl tert-butyl ((5S)-6-((1,2-dithian-4-yl)amino)-6-oxohexane-1,5-diyl)dicarbamate (7, 652 mg, 1.11 mmol, 64.1%) LCMS: tᵣ=2.32 min; m/z=608.05 [M+Na]*). The disulfide intermediate was then re-dissolved in 30% diethylamine/DCM (4 mL) and stirred at room temp for another 2 h. After 2 h, solvents were removed by vacuum and the crude product was re-dissolved in DMSO/water and purified by preparative HPLC to provide the title compound as a white solid (8, 462 mg, 1.00 mmol, 89.9%). Analytical UPLC-MS (BEH C18 General Method): tᵣ=1.39 min; m/z=386.07 [M+Na]*.

### Example 3

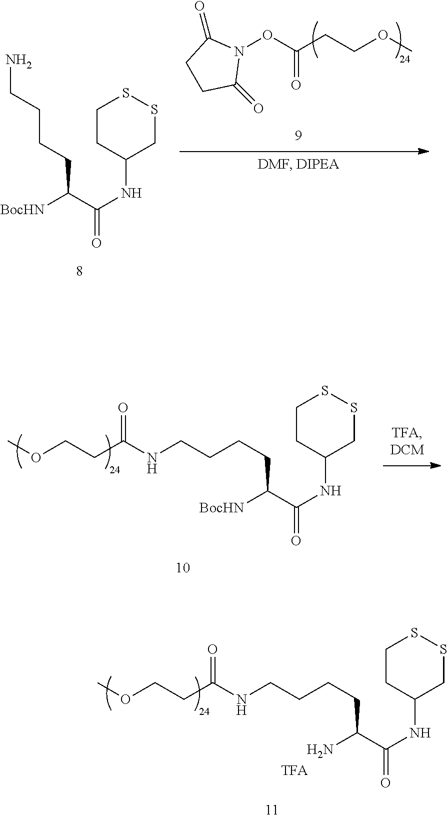

N-((5R)-6-((1,2-dithian-4-yl)amino)-5-amino-6-oxohexyl)-2,5,8,11,14,17,20,23,26,29,32,35,38,41,44,47,50,53,56,59,62,65,68,71-tetracosaoxatetraheptacontan-74-amide 2,2,2-trifluoroacetate: tert-butyl ((2S)-1-((1,2-dithian-4-yl)amino)-6-amino-1-oxohexan-2-yl)carbamate (8, 102.2 mg, 0.281 mmol) was dissolved anhydrous DMF (3 mL) containing DIPEA (98 uL, 0.562 mmol). PEG₂₄-OSu (9, 375.5 mg, 0.309 mmol) was then added as white solid. The reaction mixture was stirred at room temperature for 3 h. After 3 h, the solvent was removed and the crude product was purified by silica gel chromatography (5% MeOH in DCM) to provide the disulfide intermediate compound (10, 295.2 mg, 0.202 mmol, 71.8%). Analytical UPLC-MS (BEH C18 General Method): tᵣ=1.81 min; m/z=1463.42 [M+H]*. Compound 10 (108 mg, 0.074 mmol) was then re-dissolved in 10% TFA/DCM and stirred at room temp for 30 mins. After 30 mins, the solvent was removed, and the title compound (11) as the crude product was used directly for the next step without purification. Analytical UPLC-MS (BEH C18 General Method): tᵣ=1.33 min; m/z=1363.28 [M+H]*.

### Example 4

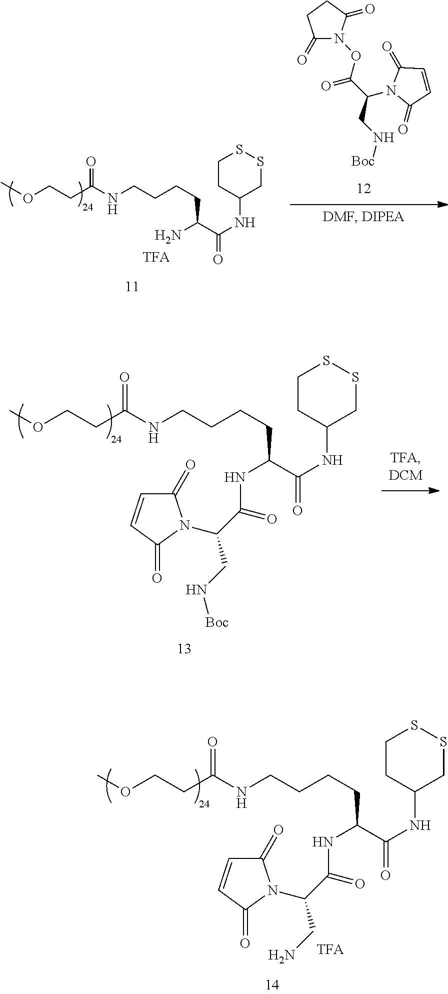

N-((5S)-6-((1,2-dithian-4-yl)amino)-5-((S)-3-amino-2-(2,5-dioxo-2,5-dihydro-1H-pyrrol-1-yl)propanamido)-6-oxohexyl)-2,5,8,11,14,17,20,23,26,29,32,35,38,41,44,47,50,53,56,59,62,65,68,71-tetracosaoxatetraheptacontan-74-amide: The amine TFA salt (11, 109 mg, 0.074 mmol) was dissolved in anhydrous DMF (1 mL) followed by the addition of DIPEA (38.6 μL, 0.221 mmol). 2,5-Dioxopyrrolidin-1-yl (S)-3-((tert-butoxycarbonyl)amino)-2-(2,5-dioxo-2,5-dihydro-1H-pyrrol-1-yl)propanoate (12, 25.3 mg, 0.066 mmol) in anhydrous DMF (0.1 mL) was then added. The reaction mixture was stirred at room temp for 2 h. After 2 h, the reaction was acidified with HOAc (40 μL), diluted with DMSO/water and purified by prep-HPLC to provide the maleimide intermediate (13, 101.1 mg, 0.062 mmol, 84.0%). Analytical UPLC-MS (General Method): tᵣ=1.73 min; m/z=1628.95 [M+H]*. Compound 13 (101.1 mg, 0.062 mmol) was re-dissolved in 10% TFA in DCM and stirred at room temp for 90 mins. After 90 mins, the solvent was removed and the crude product was dissolved in DMSO/water and purified by preparative HPLC to provide the titled compound as a colorless liquid (14, 79.1 mg, 0.048 mmol, 77.6%). Analytical UPLC-MS (BEH C18 General Method): tᵣ=1.36 min; m/z=1529.40 [M+H]⁺.

### Example 5

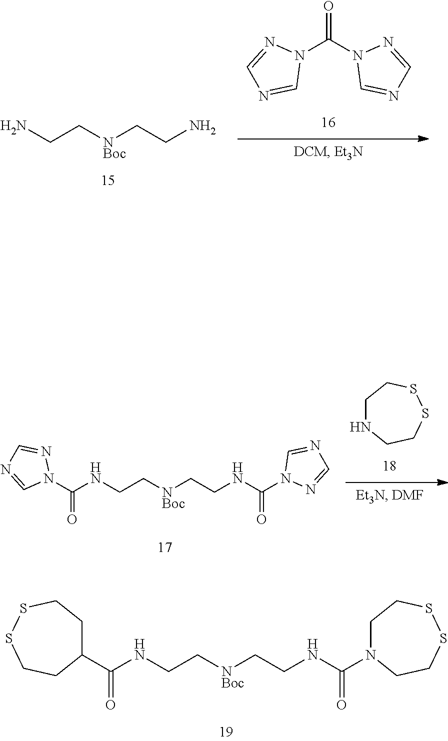

tert-butyl bis(2-(1,2,5-dithiazepane-5-carboxamido)ethyl)carbamate: tert-butyl bis(2-aminoethyl)carbamate (15, 220 mg, 1.08 mmol) and di(1H-1,2,4-triazol-1-yl)methanone (16, 1.07 g, 6.49 mmol) were dissolved in DCM (10 mL) followed by the addition of triethylamine (0.30 mL, 2.16 mmol). The reaction mixture was stirred at room temp for 30 mins. After 30 mins, LCMS indicated the full conversion of the diamine. The solvent was removed, and the crude product was purified by silica gel chromatography (0-5% MeOH in DCM) to provide tert-butyl bis(2-(1H-1,2,4-triazole-1-carboxamido)ethyl)carbamate (17, 379.0 mg, 0.963 mmol, 89.0%) as a light yellow solid. Analytical UPLC-MS (BEH C18 General Method): tᵣ=1.31 min; m/z=394.22 [M+H]+. Compound 17 (201 mg, 0.511 mmol) was re-dissolved in DMF (3 mL) followed by the addition of trimethylamine (0.21 mL, 1.53 mmol) and 1,2,5-dithiazepane (141.6 mg, 1.05 mmol). The reaction mixture was heated up to 45 degrees for 5 h. After 5 h, the reaction was cooled to room temp. The solvent was removed under reduced pressure and the crude product was purified by silica gel chromatography (0-7% MeOH in DCM) to provide the titled bis-disulfide compound as light yellow solid (19, 233.3 mg, 0.444 mmol, 86.8%). Analytical UPLC-MS (BEH C18 General Method): tᵣ=1.91 min; m/z=525.97 [M+H]*.

### Example 6

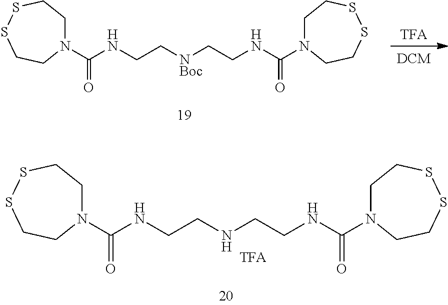

N,N′-(azanediylbis(ethane-2,1-diyl))bis(1,2,5-dithiazepane-5-carboxamide) 2,2,2-trifluoroacetate: tert-butyl bis(2-(1,2,5-dithiazepane-5-carboxamido)ethyl)carbamate (19, 135.2 mg, 0.257 mmol) was dissolved in 20% TFA/DCM (3 mL) and the reaction mixture was stirred at room temp for 1 h. After 1 h, the solvent was removed by vacuum and the titled compound (20) as the crude product was used directly for the next step. Analytical UPLC-MS (BEH C18 General Method): tᵣ=1.17 min; m/z=426.04 [M+H]*.

### Example 7

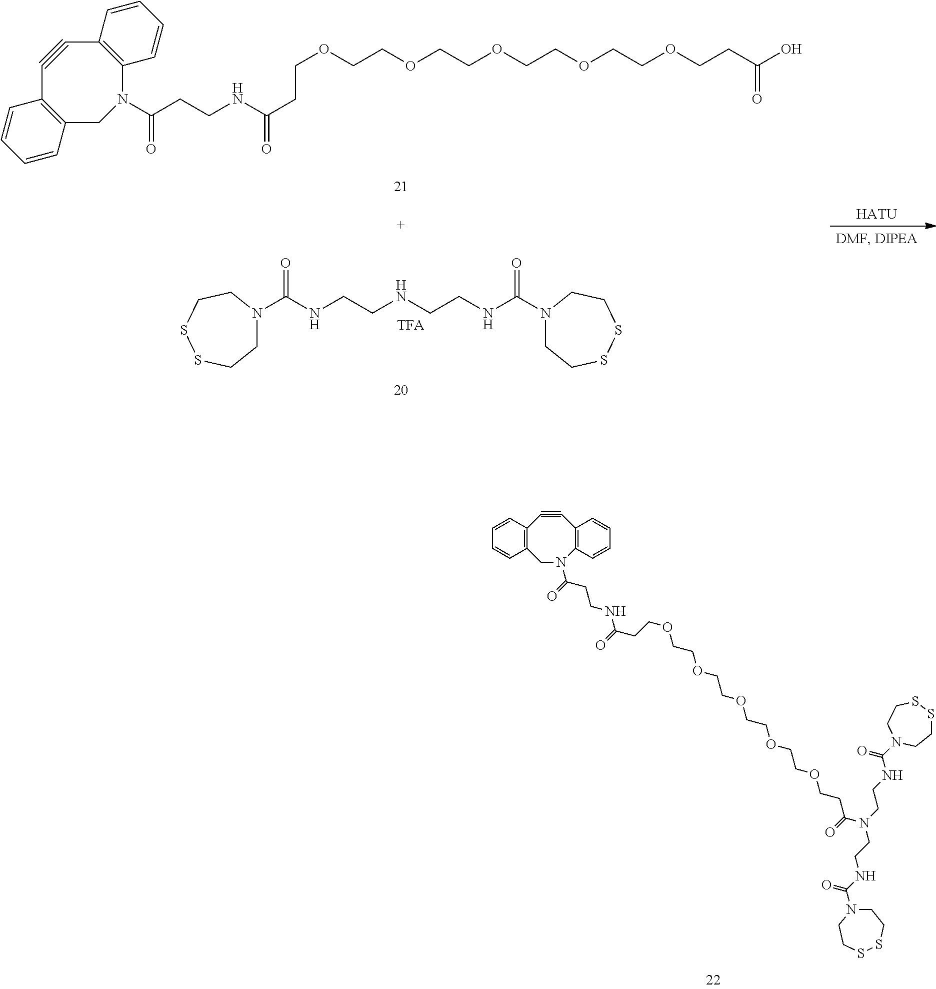 [SMILES: O=C(O)CCOCCOCCOCCOCCOCCC(=O)NCCC(=O)N1Cc2ccccc2C#Cc2ccccc21 ; "HATU" ; "DMF, DIPEA" ; O=C(NCCNCCNC(=O)N1CCSSCC1)N1CCSSCC1 ; O=C(CCOCCOCCOCCOCCOCCC(=O)N(CCNC(=O)N1CCSSCC1)CCNC(=O)N1CCSSCC1)NCCC(=O)N1Cc2ccccc2C#Cc2ccccc21]

DBCO-PEG₅-acid (21, 20.8 mg, 0.035 mmol, Broadpharm) was dissolved in anhydrous DMF (200 μL) followed by the addition of HATU (13.9 mg, 0.036 mmol) and DIPEA (0.018 mL, 0.1 mmol). After stirring at room temp for 5 mins, the disulfide amine salt (20, 17.9 mg, 0.033 mmol) in 100 μL DMF was then added. The reaction mixture was stirred at room temp for 30 mins. After 30 mins, the crude product was diluted in DMSO/water and purified by preparative HPLC to provide the DBCO bis-disulfide compound as white solid (22, 22.7 mg, 0.023 mmol, 68.1%). Analytical UPLC-MS (BEH C8 General Method): tᵣ=2.00 min; m/z=1004.18 [M+H]⁺.

### Example 8

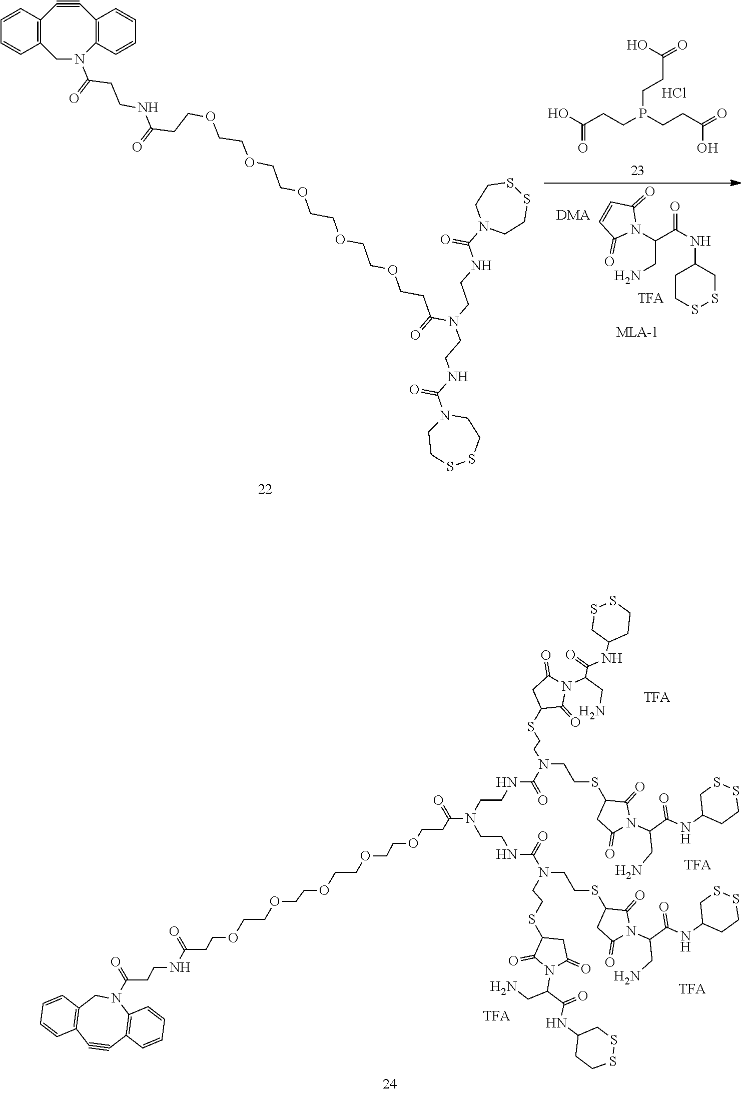

The DBCO-bis-disulfide compound (22, 50 mM in DMA, 20 μL, 1.0 μmol) was dissolved in 20 μL of 1:1 DMA/water followed by the addition of DTPA (500 mM, 1.5 μL), pH 8.0 Tris-buffer (1 M, 6 μL) and TCEP (23, 105 mM, 21 μL). The reaction mixture was warmed to 37 degrees for 2 h. After 2 h, LCMS indicated all disulfide bonds of compound 22 were reduced. The Multiplexer Linker Assembly compound of Example 1 (MLA-1, 200 mM in DMA, 35 μL, 7 μmol) was then added and the reaction temperature was maintained at 37 degrees for another 30 mins. After 30 mins, LCMS indicated the full conversion of the starting material. The crude reaction mixture was diluted with DMSO and purified by preparative HPLC to provide the DABCO-tetrakis disulfide compound as a white solid (24, 1.18 mg, 0.442 umol, 44.2%). Analytical UPLC-MS (BEH C18 Hydrophobic Method): tᵣ=1.00 min; m/z=1107.51 [1/2M+H]⁺. Compound 24 is an exemplary MLA compound of Formula II

### Example 9

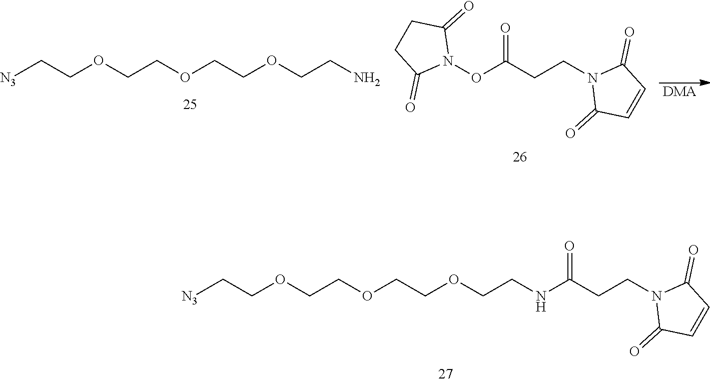

The azido-amine (25, 2.26 mg, 10.3 uM, Broadpharm) was dissolved in DMA (0.25 mL) and added to another vial containing maleimide-OSu (26, 2.5 mg, 9.39 uM). The reaction was stirred at room temp for 30 mins. After 30 mins, LCMS indicated that compound 26 was consumed and the desired azido-maleimide compound (27) was formed. The solution (37.6 mM base on maleimide) was used directly without further purification for reaction with a cysteine thiol of an antibody or antigen-binding fragment of an antibody as described by example 11. Analytical UPLC-MS (BEH C18 General Method): tᵣ=1.26 min; m/z=370.16 [M+H]*.

The azido functional groups displayed by the antibody are capable of reacting with the strained alkyne of the bis disulfide compound (22) of example 8 or the tetrakis disulfide compound (24) of example 9 through Click Chemistry (e.g., see Chio, T. I. and Bane, S. L. in Antibody Dr Conjugates: Methods and Protocols; Method in Molecular Biology, Tumey, L. N. (ed.), 2002, vol. 2078, Chap. 6, Springer Nature) When a fully reduced antibody and the MLA compound of example 8 is used a total of 8×2 cyclic disulfides will be displayed by the antibody that are capable of providing a total 32 thiol functional groups for Drug Moiety attachment. When the MLA compound of example 9 is used 8×4 cyclic disulfides will be displayed by the antibody to provide a total of 64 thiol functional groups for Drug Moiety attachments.

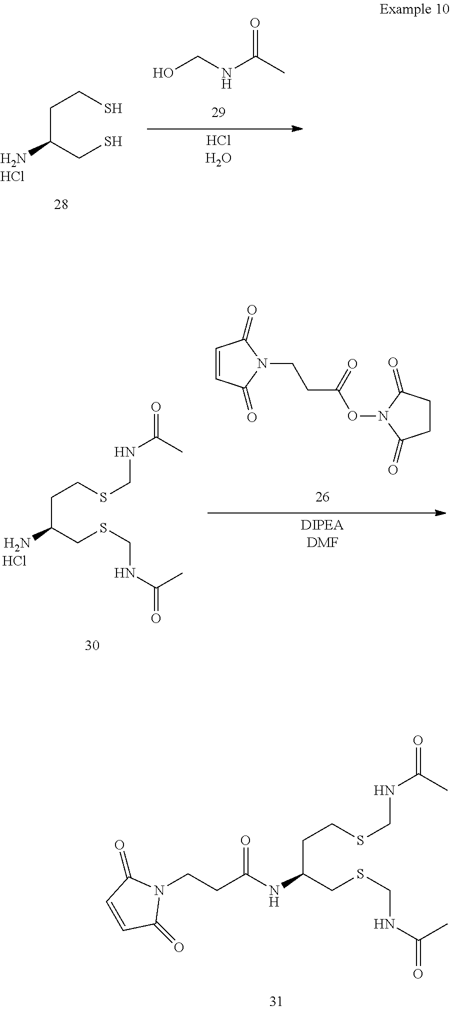

(S)-N,N′-(((2-(3-(2,5-dioxo-2,5-dihydro-1H-pyrrol-1-yl)propanamido)butane-1,4-diyl)bis(sulfanediyl))bis(methylene))diacetamide. A vial was charged with (S)-2-aminobutane-1,4-dithiol hydrochloride (28, 200 mg, 1.15 mmol) and N-(hydroxymethyl)acetamide (29, 308 mg, 3.45 mmol) and suspended in water (0.6 mL). The suspension was cooled in an ice water bath and hydrochloric acid (11.7 M, 0.2 mL, 2.34 mmol) was added dropwise. The reaction was slowly warmed to room temperature. After stirring overnight, the reaction was concentrated at 45° C. to afford the intermediate (S)-N,N′-(((2-aminobutane-1,4-diyl)bis(sulfanediyl))bis-(methylene))-diacetamide hydrochloride (30) as a clear semi-solid that was used without further purification. Analytical UPLC-MS (BEH C18 General Method): tᵣ=0.57 min, m z (ES+) calculated 280.1 (M+H)⁺, found 280.0. Compound 30 (232 mg, 0.73 mmol) and 2,5-dioxopyrrolidin-1-yl 3-(2,5-dioxo-2,5-dihydro-1H-pyrrol-1-yl)propanoate (26, 391 mg, 1.47 mmol) were combined in a vial, dissolved in DMF (2.5 mL), and DIPEA (0.51 mL, 2.94 mmol) was added dropwise. After stirring for 2 h at room temperature the reaction was quenched with acetic acid (0.25 mL), diluted with methanol and purified by preparative HPLC and lyophilized to dryness to provide the title compound (31, 42 mg, 13.3%). Analytical UPLC-MS (General Method): tᵣ=0.89 min, m z (ES+) calculated 431.1 (M+H)+, found 431.1; calculated 453.1 (M+Na)*, found 453.0.

### Example 11

### General Preparation and Analytical Characterization of an Antibody-Multiplexer Drug Conjugate.

Preparation: The following provides a method for preparing an Antibody-Multiplexer Drug Conjugate of formula I^{Ab} prepared from a formula Multiplexer Linker Assembly compound of example 1 (MLA-1) A humanized non-binding control IGg₁ antibody is treated with an excess of reductant to fully reduce its interchain disulfide bonds, which results in 8 available cysteine residues for Michael addition to the maleimide moiety of MLA-1, according the procedure of US 2003/00883263. Briefly, the non-binding antibody (5-10 mg/mL) in phosphate buffered saline with 1 mM ethylenediaminetetraacetic acid (EDTA) was treated with 10 eq. tris(2-carboxyethyl)phosphine (TCEP) neutralized to pH 7.4 using potassium phosphate dibasic and incubated at 37 C for 45 minutes. Separation of low molecular weight components was achieved by size exclusion chromatography on a Sephadex G25 column.

The fully reduced antibody was then treated with an excess of MLA-1 analogous to the procedures of US 2005/0238649. Briefly, MLA-1 in DMSO was added to the fully reduced non-binding antibody in PBS with EDTA along with excess DMSO to a total reaction co-solvent of 15% v/v. After 30 minutes at ambient temperature, an excess of n-acetyl cysteine was added to the mixture to quench all unreacted maleimide groups. The reaction mixture was purified by desalting using Sephadex G25 resin into PBS buffer. From fully reducing the interchain disulfides of a human IgG1 antibody and reaction of the maleimide to the resulting cysteines, each light chain of the antibody will have a single maleimide modification and each heavy chain will contain three maleimide modifications in which the maleimide moiety of MLA-1 has been converted to a thio-substituted succinimide moeity. Due to the presence of the —CH₂NH₂ substituent in MLA-1 the succinimide ring will undergo spontaneous hydrolysis in the formula I^{Ab} conjugate before and/or after reduction of the disulfide bonds of the MLA Units to provide a ring-opened form as described more generally by WO 2013/173337.

Analytical Characterization: For simplicity, FIG. 1 shows modification of the cysteine residue of one of the antibody's light chains by MLA-1. The m/z of the light chain prior to reaction with MLA-1 is 23152 AMU and immediately after reaction with MLA-1 it is expected to increase to 23476 AMU (see panel A). The cyclic disulfide bond of each of the eight MLA Unit in the Ab-MLA intermediate was then reduced with TCEP for display of a total of 16 reactive thiol functional groups. Those thiols were then reacted with N-ethyl-maleimide (NEM), which serves as a dummy Drug Moiety precursor, in the same manner as previously described for reaction of the antibody cysteine thiols with MLA-1. During that time the succinimide ring will have undergone spontaneous hydrolysis. However, that hydrolysis may have occurred prior to the NEM reaction since the shown Na⁺ adduct in FIG. 1 would be indistinguishable from the open form of the initially formed succinimide Ab-MLA adduct due to insufficient resolution of the mass spectrometer. The resulting light chain conjugate in which two dummy NEM Drug Moieties have been covalently attached to a single cysteine thiol through intermediacy of one Multiplexer Linking Assembly Unit is expected to show a m/z of 23723 AMU (see Panel B). The structure characterized by Panel B is analogous to a TM-ADC of Formula I^{Ab}.

### Example 12

### Preparation and Analytical Characterization of Auristatin Thiol Multiplexer Antibody Drug Conjugates

Preparation: An Auristatin Thiol Multiplexed Antibody Drug Conjugate (cAC10-MLA1-D^{M1}) was prepared according to the generalized procedure of example 11 in which the non-binding control antibody is replaced with CD30-binding chimeric antibody cAC10 and the NEM “dummy” Drug Moiety precursor is replaced with the PEGylated Auristatin Drug Moiety (D^{M1}) precursor having the structure of:

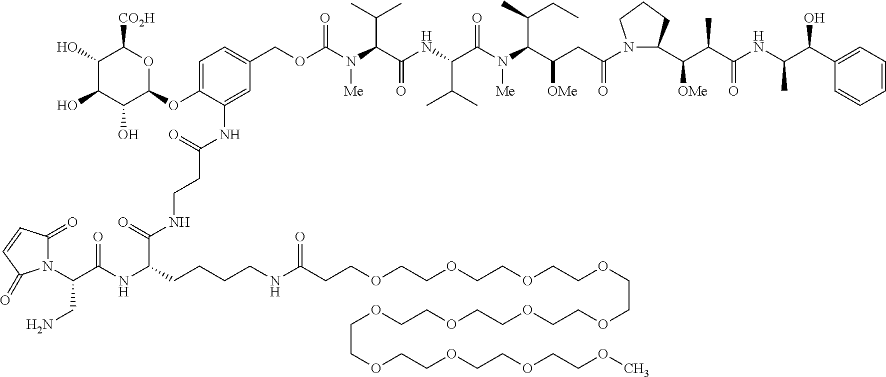 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCNC(=O)[C@H](CCCCNC(=O)CCOCCOCCOCCOCCOCCOCCOCCOCCOCCOCCOCCOC)NC(=O)[C@H](CN)N2C(=O)C=CC2=O)c1)C(C)C]

The Pegylated Auristatin Drug Moiety precursor was prepared according to the procedures of WO 2015/057699 and WO 2016/149535.

Another Auristatin Thiol Multiplexed Antibody-Drug Conjugate (cAC10-MLA1-D^{M2}) was prepared according to the generalized procedure of example 11 using cAC10 and the hydrophilic Auristatin Drug Moiety precursor having the structure of:

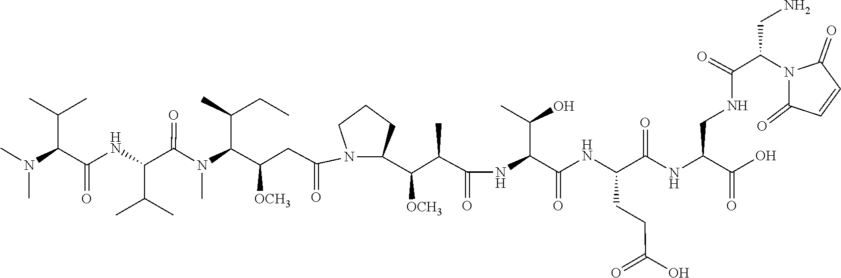 [SMILES: CC[C@H](C)[C@@H]([C@@H](CC(=O)N1CCC[C@H]1[C@H](OC)[C@@H](C)C(=O)N[C@H](C(=O)N[C@@H](CCC(=O)O)C(=O)N[C@@H](CNC(=O)[C@H](CN)N1C(=O)C=CC1=O)C(=O)O)[C@@H](C)O)OC)N(C)C(=O)[C@@H](NC(=O)[C@H](C(C)C)N(C)C)C(C)C]

The hydrophilic Auristatin Drug Moiety precursor (D^{M2}) was prepared according to the procedures of WO 2015/123679.

Characterization: Size-Exclusion Chromatography (SEC) for the 16-load auristatin ADCs based on MDL-1 of Example 11 shows low percentages of high molecular weight species (2% for cAC10-MLA1-D^{ML} and cAC10-MLA1-D^{M2}).

PLRP chromatography (FIG. 2, Panel A): L-MLA1-D^{M1}(2) tᵣ=1.29 min; H-MLA1-D^{M1}(6) tᵣ=1.97 min. Each light and heavy chain incorporates 1 and 3 molecules of PEGylated MLA1-D^{M1}, respectively, so that each light chain is conjugated to a total of 2 auristatin Drug Units and each heavy chain is conjugated to a total of 6 PEGylated auristatin Drug Units. Mass spectroscopy of the cAC10 light chain, which is attached to one molecule of MLA-1, is shown in Panel B of FIG. 2. Mass spectroscopy of the cAC10 light chain attached to one molecule of PEGylated MLA1-D^{M1}, and hence having conjugation to two auristatin Drug Units, is shown in Panel C of FIG. 2. Mass spectroscopy of the cAC10 heavy chain conjugated to 3 molecules of PEGylated MLA1-D^{M1}, and hence having conjugation to 6 auristatin Drug Units, is shown in Panel D of FIG. 2. Multiple peaks in Panel D are due to G0, G1 and G2 oligosaccharide forms of the heavy chain.

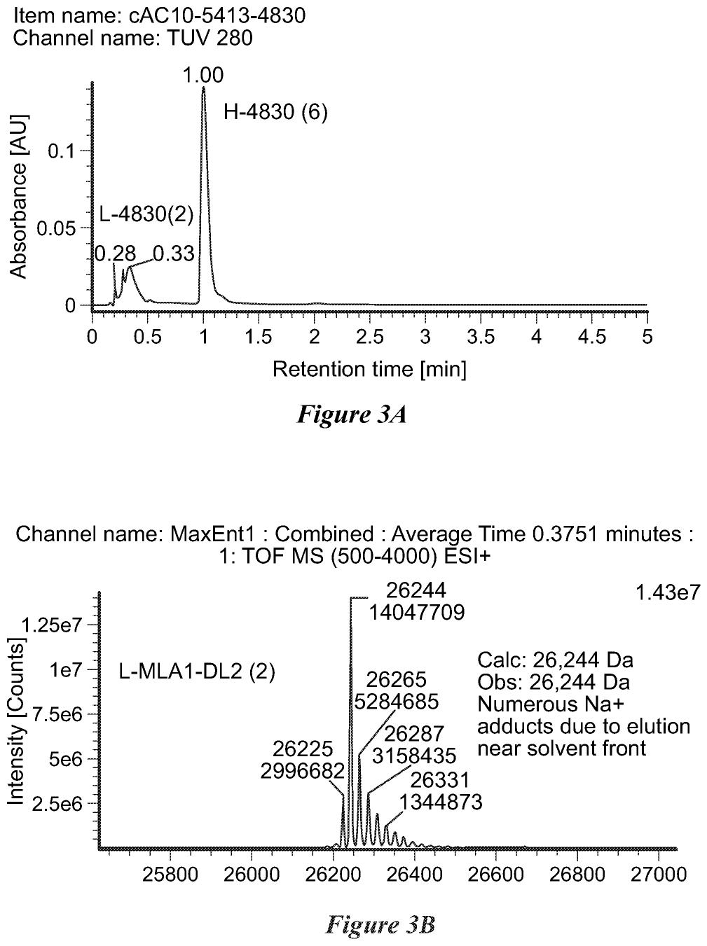

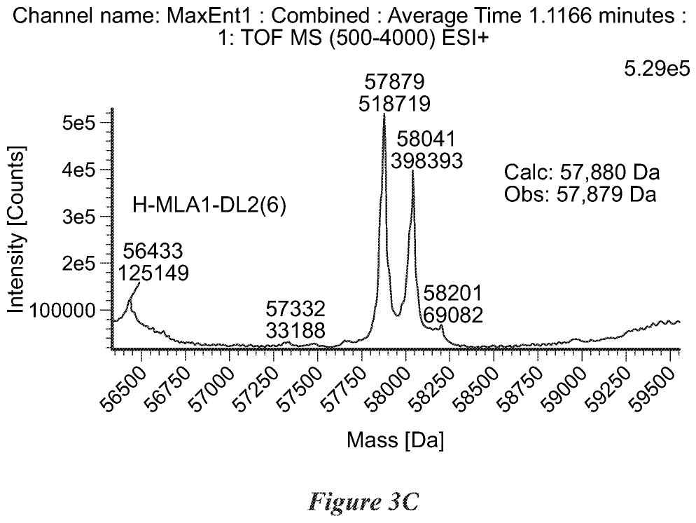

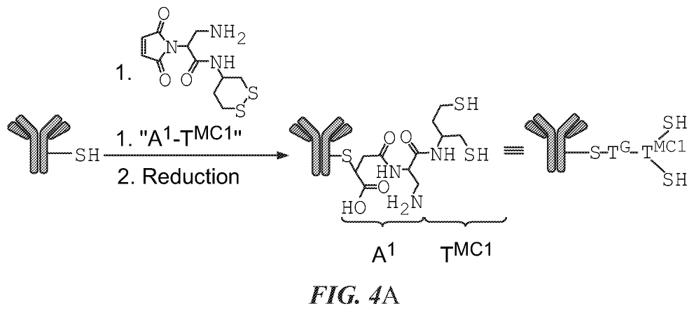

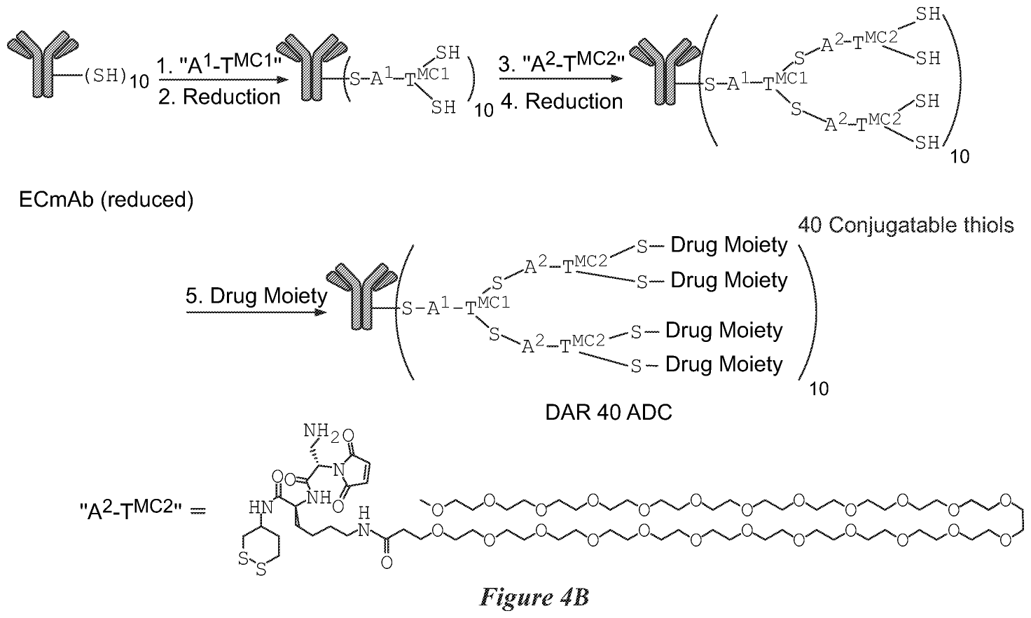

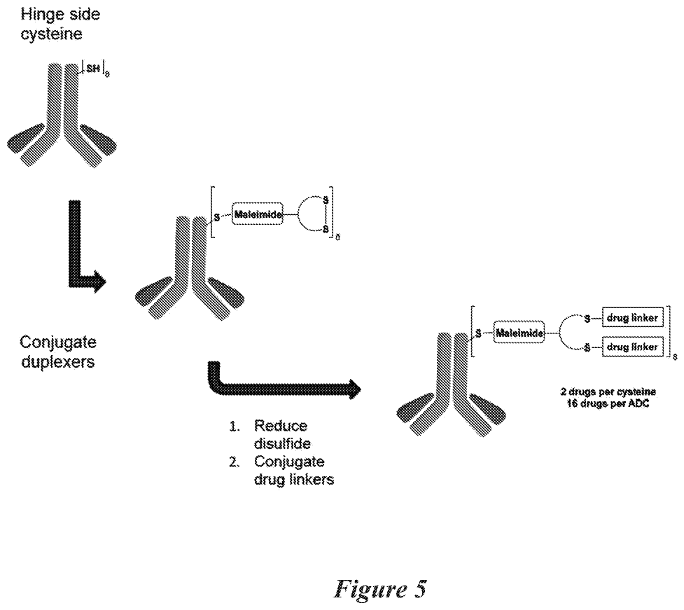

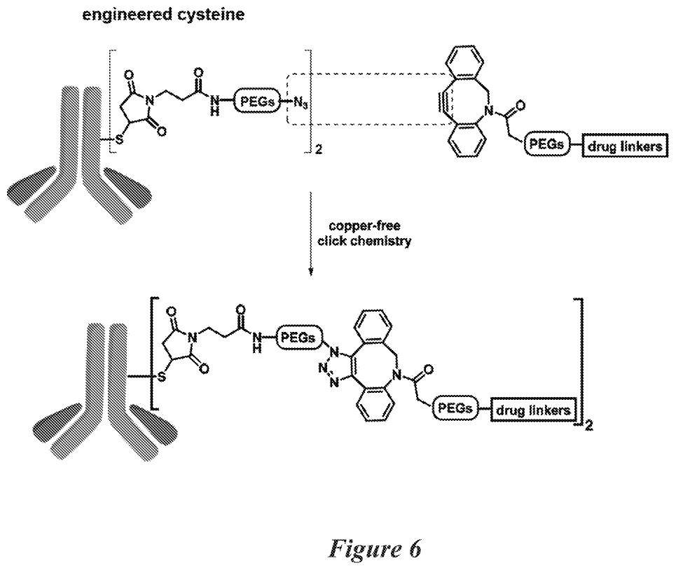

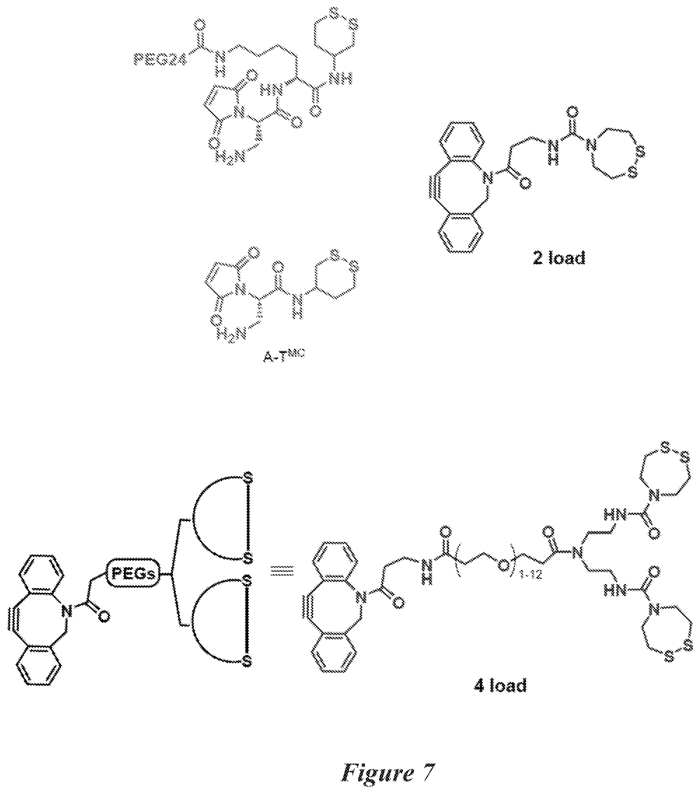

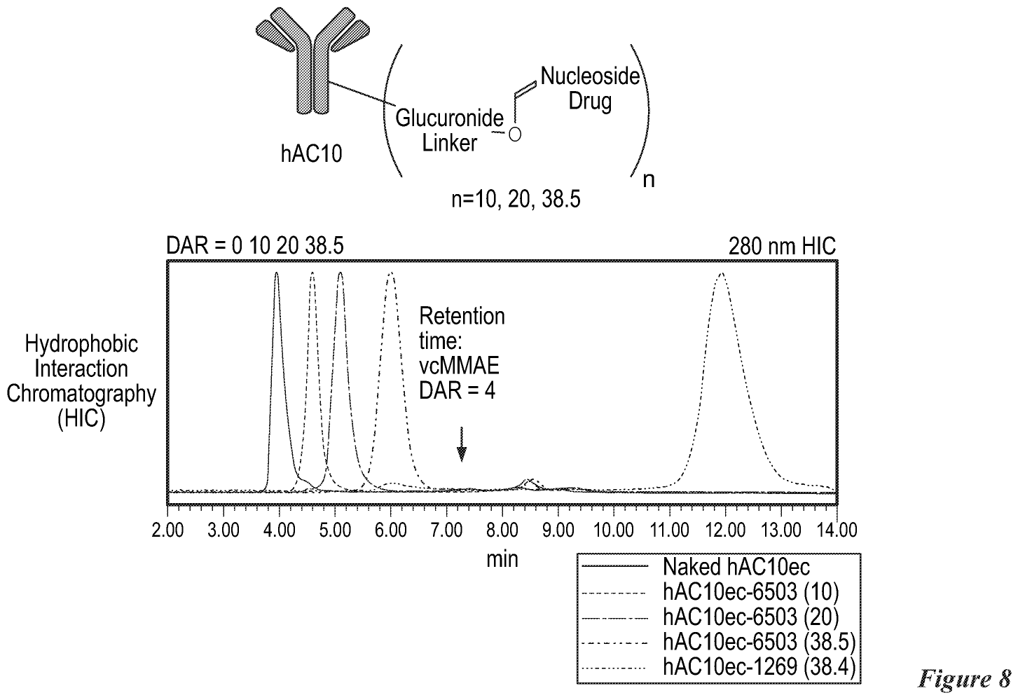

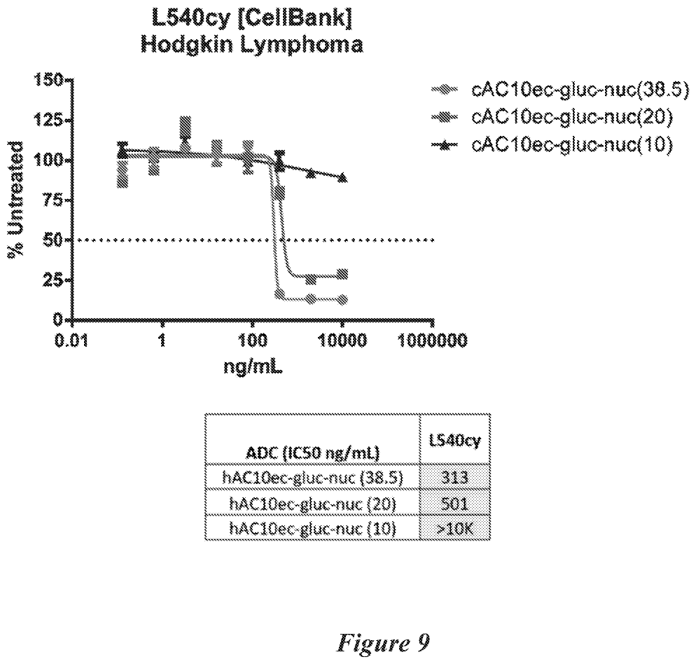

PLRP chromatography (FIG. 3, Panel A): L-MLA1-D^{M2}(2) tᵣ=0.33 min; H-MLA1-D^{M2}(6) tᵣ=1.00. Mass spectroscopy of the cAC10 light chain attached to one molecule of hydrophilic MLA1-D^{M2}, and hence having conjugation to two auristatin Drug Units, is shown in Panel B of FIG. 2. Mass spectroscopy of the cAC10 heavy chain conjugated to 3 molecules of hydrophilic MLA1-D^{M2}, and hence having conjugation to 6 auristatin Drug Units, is shown in Panel C of FIG. 2. Multiple peaks in Panel C are due to G0, G1 and G2 oligosaccharide forms of the heavy chain.

### Example 13

### Preparation of a Gemcitabine Drug Moiety

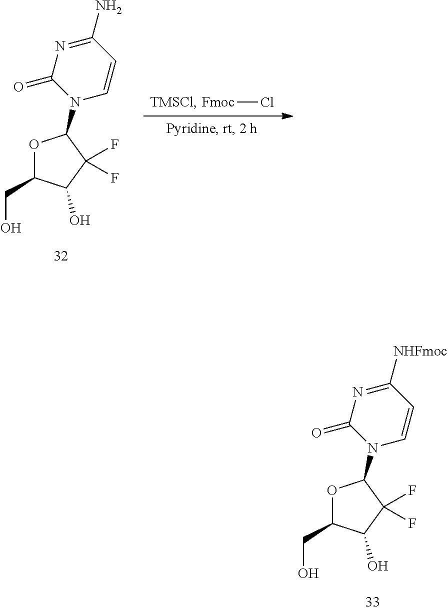

(9H-Fluoren-9-yl)methyl (1-((2R,4R,5R)-3,3-difluoro-4-hydroxy-5-(hydroxymethyl)-tetrahydrofuran-2-yl)-2-oxo-1,2-dihydropyrimidin-4-yl)carbamate: Gemcitabine (32, 782.6 mg, 2.973 mmol) was dissolved in anhydrous pyridine (10 mL). TMSCl (1.89 mL, 14.9 mmol) was added to the vigorously stirred reaction over 5 minutes. The reaction was stirred for 15 minutes and a white precipitate formed. Fmoc-Cl (961.5 mg, 3.717 mmol) was added to the reaction in one portion. The reaction turned yellow then, then colorless over 30 minutes, a white precipitate persists over the course of the reaction. H2O (2 mL) was added, and the reaction was stirred for 2 h to hydrolyze the TMS groups and excess chloroformate. The reaction mixture was diluted with EtOAc (100 mL), washed with 1M HCl (3×100 mL), dried MgSO₄, filtered and concentrated in vacuo. Crude title compound was purified by flash chromatography 100G KP-Sil 50-100% EtOAc in Hex. R_{f} (33)=0.15 (1:2 Hex:EtOAc). Fractions containing the desired product were concentrated in vacuo to afford the title compound as a white solid (33, 1.169 g, 2.407 mmol, 80.9%). tᵣ=1.71 min (CORTECS C18 General Method); MS (m/z) [M+H]⁺ calc. for C₂₄H₂₂ F₂N₃O₆ 486.45, found 486.12.

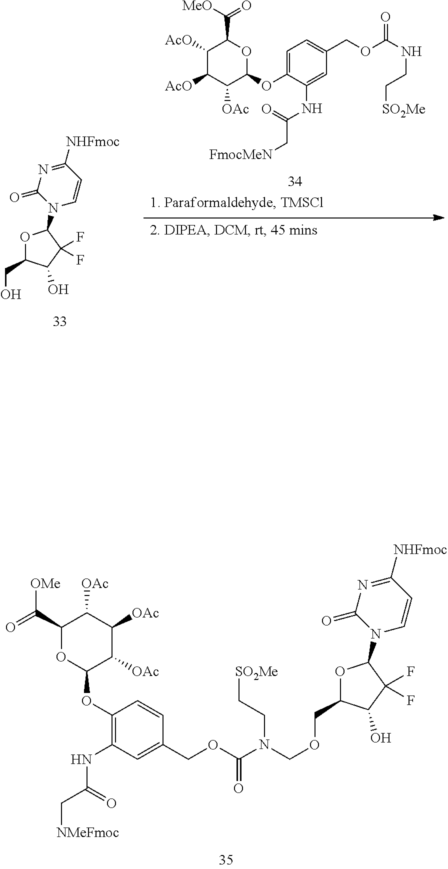

(2R,3S,4R,5R,6R)-2-(2-(2-((((9H-fluoren-9-yl)methoxy)carbonyl)(methyl)amino)-acetamido)-4-(((((((2R,3R,5R)-5-(4-((((9H-fluoren-9-yl)methoxy)carbonyl)amino)-2-oxopyrimidin-1(2H)-yl)-4,4-difluoro-3-hydroxytetrahydrofuran-2-yl)methoxy)methyl)(2-(methylsulfonyl)ethyl)-carbamoyl)oxy)methyl)phenoxy)-6-(methoxycarbonyl)tetrahydro-2H-pyran-3,4,5-triyl triacetate. The MAC linker intermediate compound (34, 185 mg, 0.206 mmol), prepared analogously to the procedure of WO 2015/095755 and by Kolakowski, R. V. et al. Angew. Chem. Int'l Ed. (2016) 55: 7948-7951, was dissolved in DCM (2 mL). Paraformaldehyde (185 mg, 6.18 mmol) was added followed by TMSCl (1 mL). The reaction was stirred for 10 minutes at which point complete conversion was observed by diluting 2 uL aliquot into 98 uL of MeOH and observing the MeOH adduct by UPLC-MS. The reaction was filtered with a syringe filter, rinsed with DCM (1 mL), and Toluene (2 mL) was added to azeotrope final mixture upon concentration. The eluent was concentrated in vacuo to afford a colorless solid. Fmoc-Gemcitabine (33) was azeotroped with toluene and died under high vacuum prior to use. Compound 33 (100 mg, 0.206 mmol) was suspended in anhydrous DCM (2 mL) and DIPEA (71.8 μL, 0.412 mmol) was added. The activated linker was dissolved in anhydrous DCM (2 mL) and added dropwise to the stirring reaction at a rate of 10 mL/h. The reaction was stirred for 45 minutes at which point complete conversion was observed. The reaction was quenched with MeOH (0.1 mL), filtered and the eluent was concentrated in vacuo to afford the title compound as colorless solid which was used in the next step without purification (35, 182 mg, 0.130 mmol, crude, 63%). tᵣ=1.56 min (CORTECS C18 Hydrophobic Method); MS (m/z) [M+H]⁺ calc. for C₆₇H₆₉F₂N₆O₂₃S 1395.41, found 1395.40.

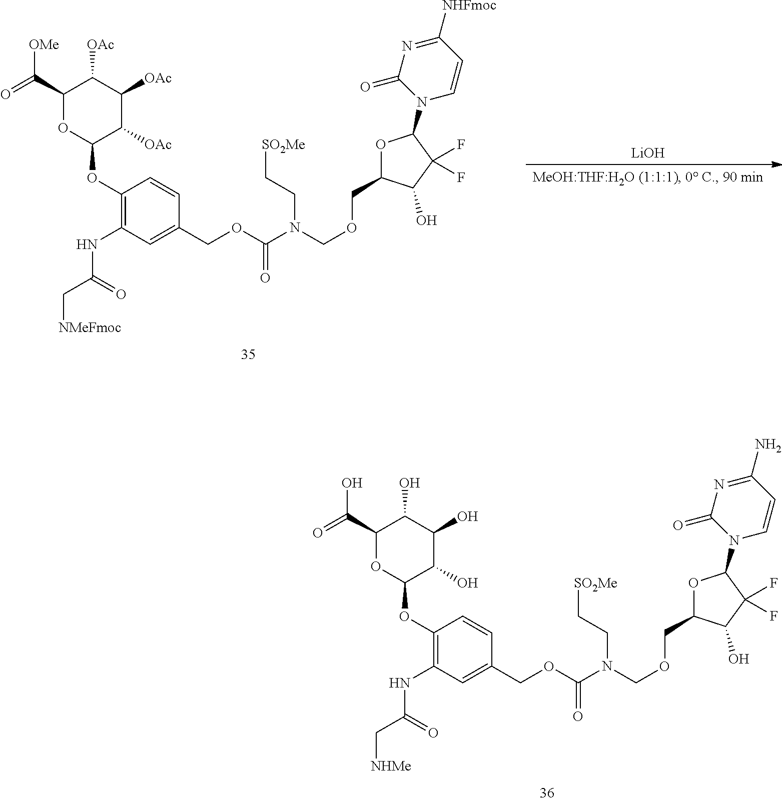

(2R,3R,4R,5S,6R)-6-(4-(((((((2R,3R,5R)-5-(4-amino-2-oxopyrimidin-1(2H)-yl)-4,4-difluoro-3-hydroxytetrahydrofuran-2-yl)methoxy)methyl)(2-(methylsulfonyl)ethyl)carbamoyl)-oxy)methyl)-2-(2-(methylamino)acetamido)phenoxy)-3,4,5-trihydroxytetrahydro-2H-pyran-2-carboxylic acid: Compound 35 (182 mg, 0.130 mmol) was dissolved in THF:MeOH 1:1 (2 mL). The reaction solution was cooled with an ice/water bath upon which LiOH (31.2 mg, 1.30 mmol) was added. The resulting reaction mixture was stirred for 30 minutes whereupon H₂O (1 mL), was then added. The reaction was then stirred for 60 minutes. Complete conversion to the deprotected compound was determined by UPLC-MS. The reaction mixture was quenched with AcOH (30 μL), concentrated in vacuo and purified by preparative HPLC using a 21.2×250 mm Max-RP column eluted with a gradient of 5-35-95% MeCN in H₂O 0.05% TFA Fractions containing the desired deprotected compound were concentrated in vacuo to afford the titled compound as a colorless solid (36, 65.1 mg, 0.0803 mmol, 62%). tᵣ=0.82 min (CORTECS C18 Hydrophilic Method); MS (m/z) [M+H]⁺ calc. for C₃₀H₄₁ F₂N₆O₁₆S 811.23, found 811.04.

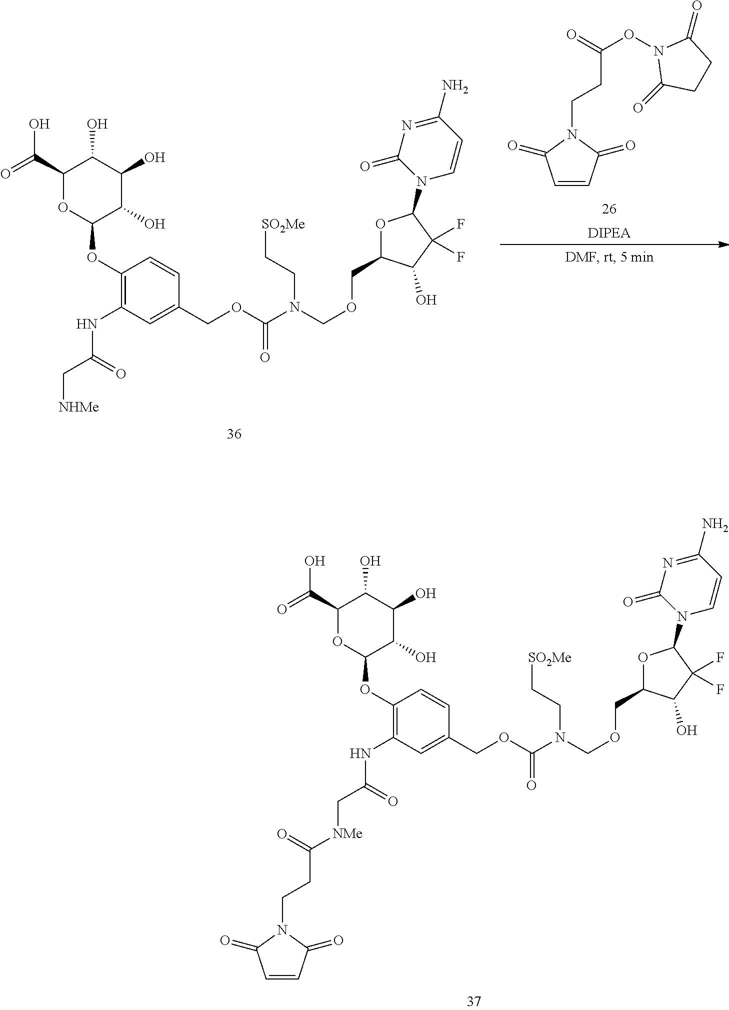 [SMILES: CNCC(=O)Nc1cc(COC(=O)N(CCS(C)(=O)=O)COC[C@H]2O[C@@H](n3ccc(N)nc3=O)C(F)(F)[C@@H]2O)ccc1O[C@H]1O[C@@H](C(=O)O)[C@H](O)[C@@H](O)[C@@H]1O ; O=C(CCN1C(=O)C=CC1=O)ON1C(=O)CCC1=O ; "DIPEA" ; "DMF, rt, 5 min" ; CN(CC(=O)Nc1cc(COC(=O)N(CCS(C)(=O)=O)COC[C@H]2O[C@@H](n3ccc(N)nc3=O)C(F)(F)[C@@H]2O)ccc1O[C@H]1O[C@@H](C(=O)O)[C@H](O)[C@@H](O)[C@@H]1O)C(=O)CCN1C(=O)C=CC1=O]

(2R,3R,4R,5S,6R)-6-(4-(((((((2R,3R,5R)-5-(4-amino-2-oxopyrimidin-1(2H)-yl)-4,4-difluoro-3-hydroxytetrahydrofuran-2-yl)methoxy)methyl)(2-(methylsulfonyl)ethyl)carbamoyl)oxy)-methyl)-2-(2-(3-(2,5-dioxo-2,5-dihydro-1H-pyrrol-1-yl)-N-methylpropanamido)acetamido)-phenoxy)-3,4,5-trihydroxytetrahydro-2H-pyran-2-carboxylic acid: Compound 36 (65.1 mg, 0.0803 mmol) was dissolved in anhydrous DMF (0.5 mL). DIPEA (26.5 μL, 0.160 mmol) was added to the reaction followed by N-Succinimidyl 3-Maleimidopropionate (26, 23.5 mg, 0.0883 mmol, purchased from TCI America product number S0427). The reaction was stirred for 15 minutes when complete conversion was observed by UPLC-MS. The reaction was quenched with AcOH (0.020 mL) and purified by preparative HPLC eluting with 5-35-95% MeCN in H₂O 0.05% TFA on a 21.2×250 mm Max-RP. Fractions containing the desired product were lyophilized to afford the title compound as a colorless powder (37, 41.2 mg, 0.0428 mmol, 53.3%). tᵣ=1.29 min (CORTECS C18 Hydrophilic Method); MS (m/z) [M+H]⁺ calc. for C₃₇H₄₆ F₂N₇O₁₉S 962.25, found 962.06.

MAL compound 37 and Ab-MLA intermediate of example 11 in which the humanized non-binding control IGg₁ antibody is replaced with the chimeric monoclonal cAC10 are treated in the manner described in preparation of the auristatin TM-ADC to provide the analogous gemcitabine TM-ADC.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

What is claimed is:

1. A Multiplexer Linking Assembly (MLA) compound, having formula (I): 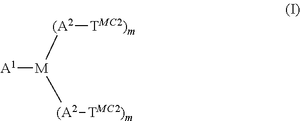
wherein:
A¹ is a first Linking Group;
each A² is independently a bond or an independently selected second linking group;
each of A¹ and A² comprises 0 or 1 Partitioning Groups (Y) that are covalently attached to the first or second Linking Groups, or a divalent linking component of, the first or second Linking Groups;
M is a Multiplexing Group or a first Thiol Multiplexing Group (T^{MC1});
each T^{MC2} is a second Thiol Multiplexing Group;
subscript m is 0 or 1; wherein:
when subscript m is 0, M is a first Thiol Multiplexing Group (T^{MC1}) in disulfide form or is a first Thiol Multiplexing Group (T^{MC1}) attached to two Drug Moieties (D^{M}); and
when subscript m is 1, each T^{MC2} is in disulfide form, or is attached to two Drug Moieties (D^{M}).

2. The MLA compound of claim 1, wherein A¹ has a formula selected from the group consisting of: 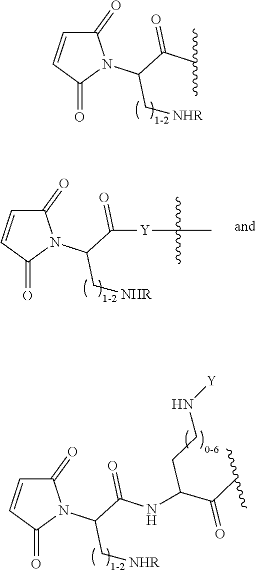
wherein R is selected from the group consisting of H and an amine protecting group; Y is a Partitioning Group; and the wavy line indicates covalent attachment to M.

3. The MLA compound of claim 2, wherein M is T^{MC1} and is selected from the group consisting of:

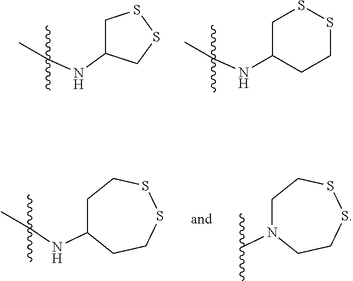 [SMILES: *NC1CSSC1 ; *NC1CCSSC1 ; *NC1CCSSCC1 ; *N1CCSSCC1]

4. The MLA compound of claim 2, wherein M is T^{MC1} and is selected from the group consisting of: 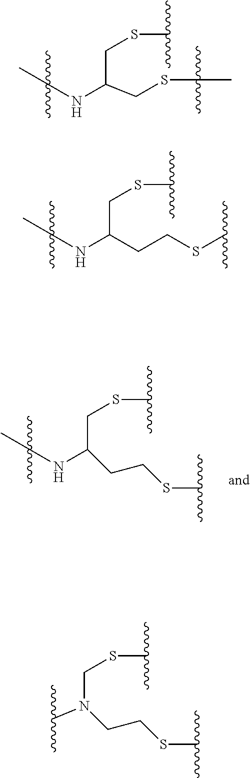 [SMILES: *NC(CS*)CS* ; *NC(CCS*)CS* ; *NC(CCS*)CS* ; *SCCN(*)CS*]
and the wavy line adjacent to sulfur indicates a site of covalent attachment to a -A²-T^{MC2} group.

5. The MLA compound of claim 4, wherein each T^{MC2} is selected from the group consisting of: 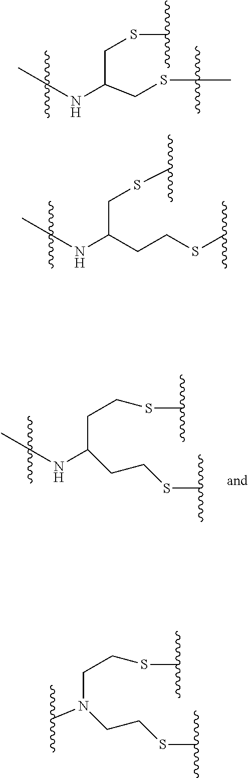 [SMILES: *NC(CS*)CS* ; *NC(CCS*)CS* ; *NC(CCS*)CCS* ; *SCCN(*)CCS*]
wherein the wavy line adjacent to each sulfur atom of each T^{MC2} indicates a site of attachment to a Drug Moiety (D^{M}).

6. The MLA compound of claim 4, wherein each T^{MC2} is selected from the group consisting of:

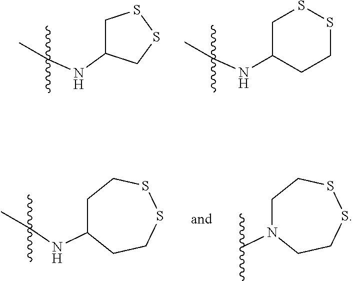 [SMILES: *NC1CSSC1 ; *NC1CCSSC1 ; *NC1CCSSCC1 ; *N1CCSSCC1]

7. The MLA compound of claim 1, wherein A¹ has a formula selected from the group consisting of: 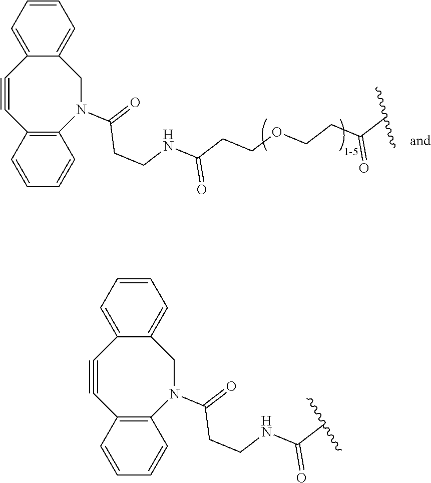
and the wavy line indicates attachment to M.

8. The MLA compound of claim 7, wherein M is T^{MC1} and is selected from the group consisting of:

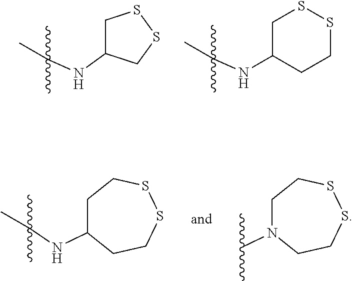 [SMILES: *NC1CSSC1 ; *NC1CCSSC1 ; *NC1CCSSCC1 ; *N1CCSSCC1]

9. The MLA compound of any one of claim 1, or 7, wherein subscript m is 1, M is T^{MC1} and is selected from the group consisting of: 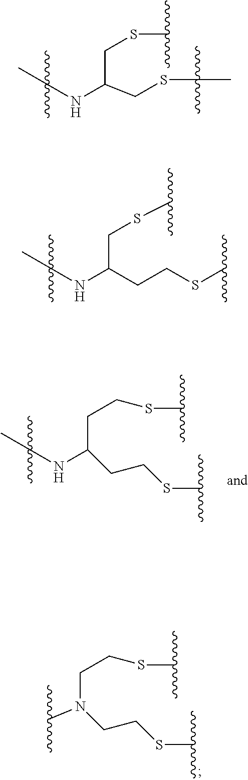 [SMILES: *NC(CS*)CS* ; *NC(CCS*)CS* ; *NC(CCS*)CCS* ; *SCCN(*)CCS*]
and T^{MC2} is selected from the group consisting of:

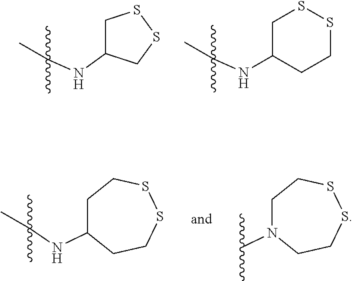 [SMILES: *NC1CSSC1 ; *NC1CCSSC1 ; *NC1CCSSCC1 ; *N1CCSSCC1]

10. The MLA compound of any one of claim 1, or 7, wherein each of (A²-T^{MC2}) has a formula independently selected from the group consisting of: 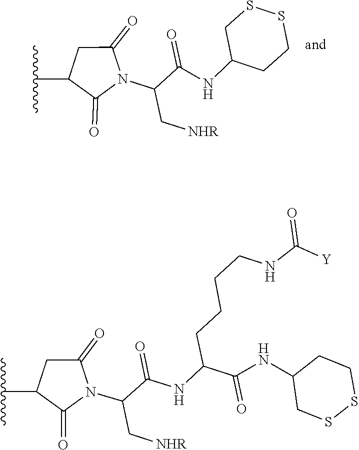
wherein the succinimide ring is in hydrolyzed form and each R is independently selected from the group consisting of H and an amine protecting group; Y is a Partitioning Group; the wavy line to the left of the succinimide ring indicates a thioether attachment to T^{MC1}.

11. The MLA compound of any one of claim 1, or 7, wherein subscript m is 0, and M is T^{MC1} and is selected from the group consisting of:

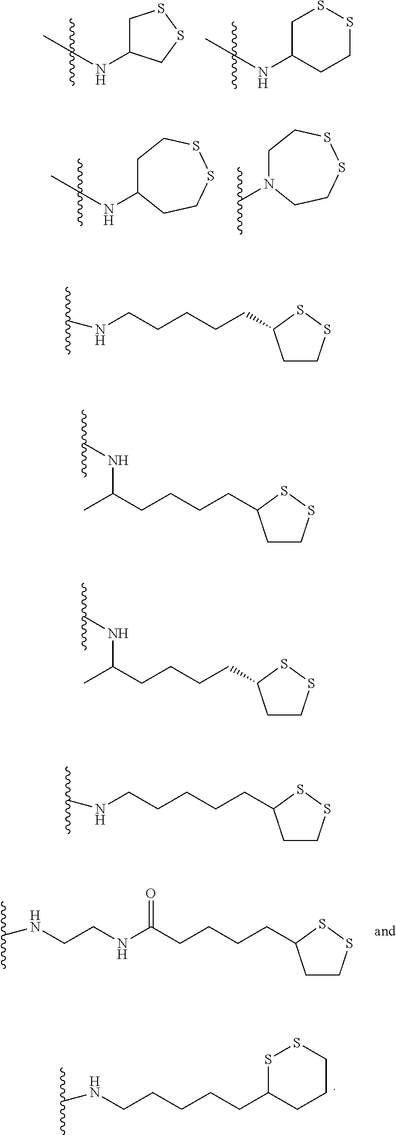

12. The MLA compound of any one of claim 1, or 7, wherein subscript m is 0 and M is T^{MC1} and is selected from the group consisting of:

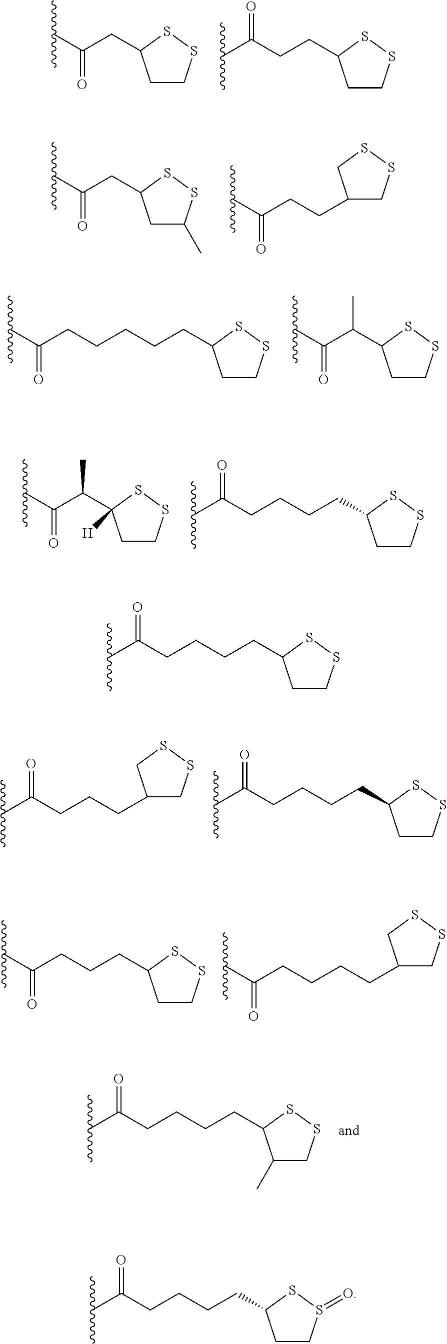

13. The MLA compound of claim 1, having a formula selected from the group consisting of I-1, I-2, I-1a, and I-2a: 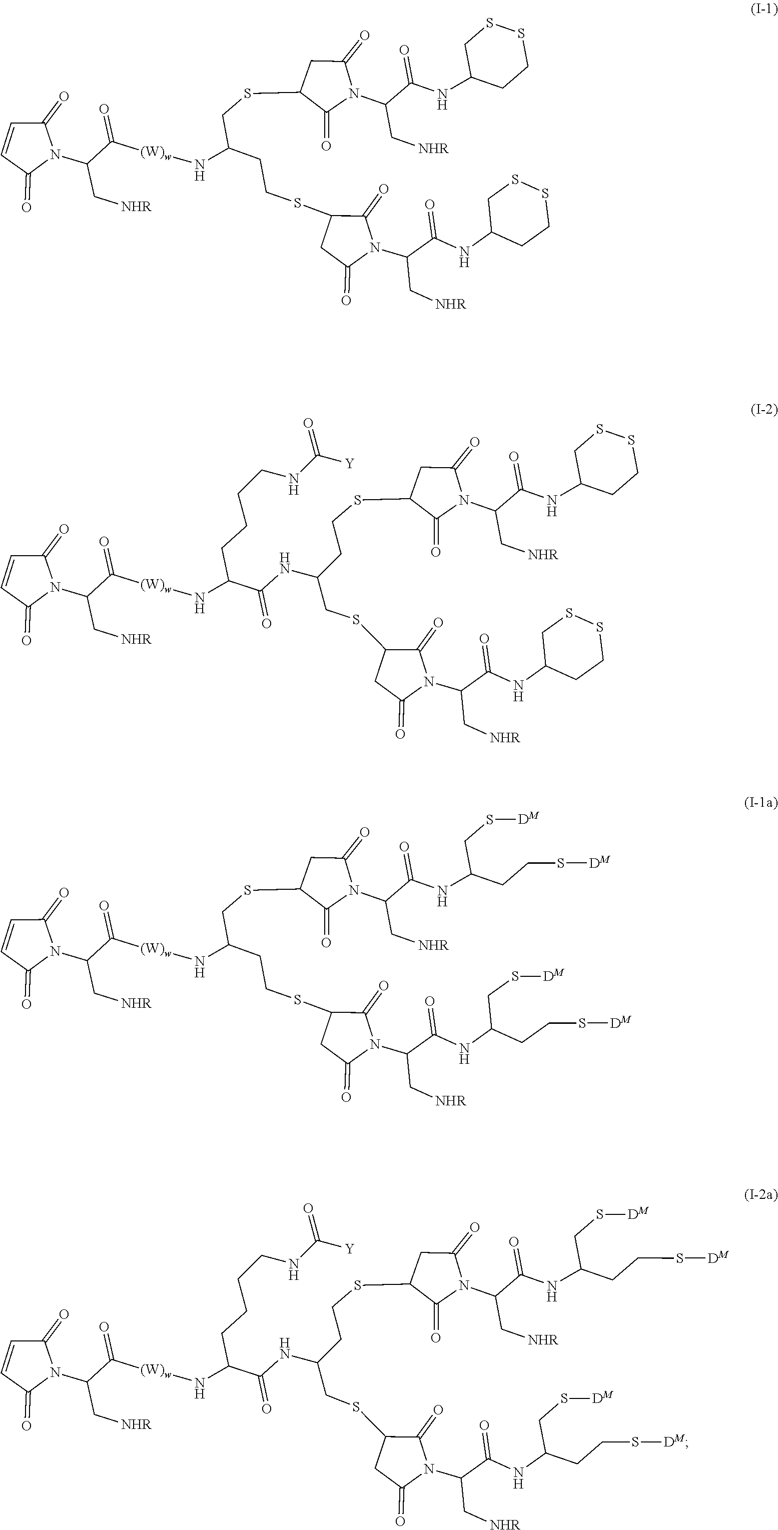
wherein
the subscript w is an integer from 0 to 8;
each W is independently a natural or non-natural amino acid;
each R is independently H or an amine protecting group; and
Y is a Partitioning Group.

14. The MLA compound of claim 13, wherein the Partitioning Group Y comprises a polyethylene glycol group.

15. The MLA compound of claim 1, having the formula selected from:

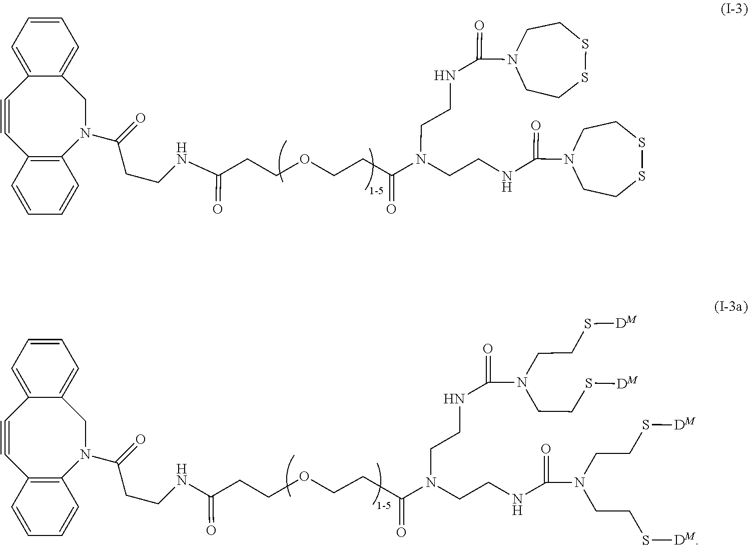

15. The MLA compound of claim 1, or 2, wherein A¹ is selected from the group consisting of a maleimide group and a halomethylcarbonyl group.

16. The MLA compound of claim 1, further comprising a Partitioning GroupGroup Y.

17. The MLA compound of claim 17, wherein the Partitioning Group Y comprises a polyethylene glycol group.

19. The MLA compound of claim 1 or 2, wherein A¹ has a formula selected from the group consisting of: 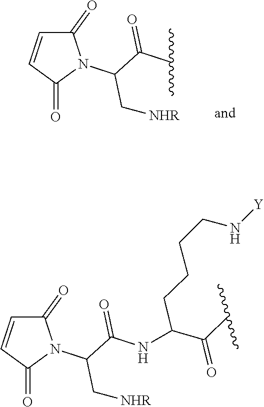
wherein R is selected from the group consisting of H and an amine protecting group; and Y is a Partitioning Group.

20. The MLA compound of claim 17, wherein the Partitioning Group is a PEG Unit selected from the group consisting of PEG₃, PEG₆, PEG₁₂, and PEG₂₄, wherein the subscripts indicate the number of repeating polyethylene glycol subunits linearly connected.

21. The MLA compound of claim 1, wherein A¹ comprises an azido or alkyne functional group capable of participating in a Click Chemistry to form a 1,2,3-triazole moiety.

22. The MLA compound of claim 1, wherein m is 1 and M is a Multiplexing Group having the structure: 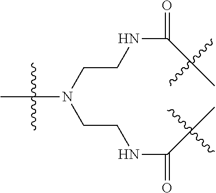 [SMILES: *C(=O)NCCN(*)CCNC(*)=O]
and each T^{MC2} is selected from the group consisting of:

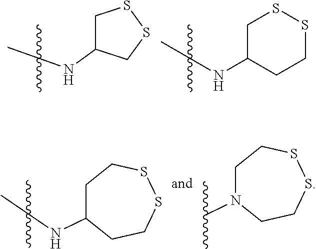 [SMILES: *NC1CSSC1 ; *NC1CCSSC1 ; *NC1CCSSCC1 ; *N1CCSSCC1]

18. The MLA compound of claim 1, wherein A¹-M- has the formula:

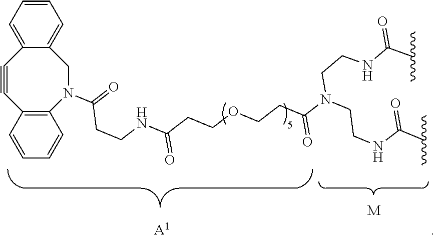

19. The MLA compound of claim 1, wherein each A² is a bond, and A¹-M-(T^{MC2})₂ has the formula:

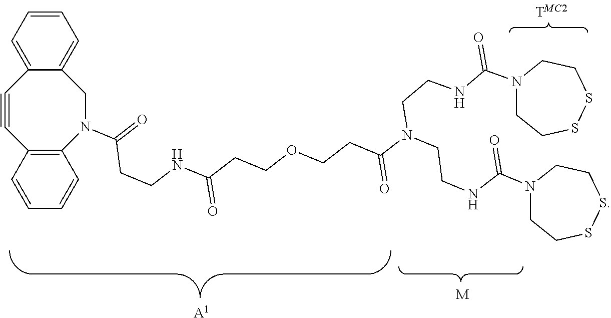

20. A compound having formula (i) or (ii): 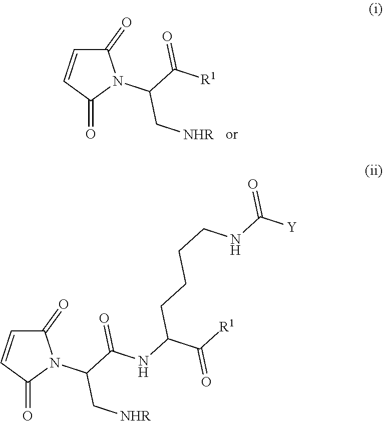
or a salt thereof, wherein R is H or an amino protecting group, Y is a Partitioning Group, and R¹ is selected from the group consisting of:

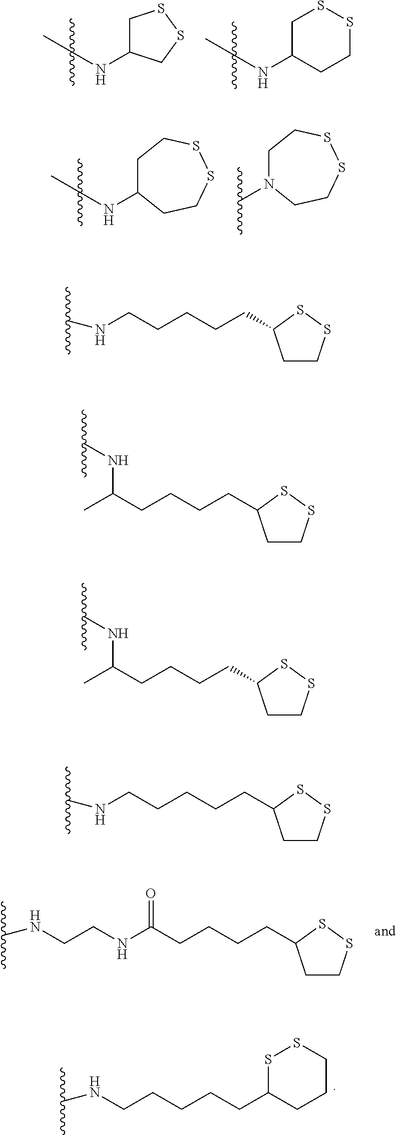

21. The compound of claim 25, wherein R¹ is selected from the group consisting of

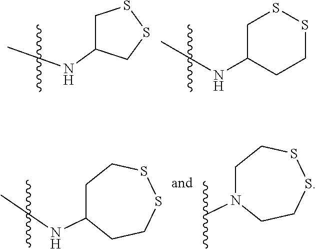 [SMILES: *NC1CSSC1 ; *NC1CCSSC1 ; *NC1CCSSCC1 ; *N1CCSSCC1]

22. The compound of claim 25, having the formula:

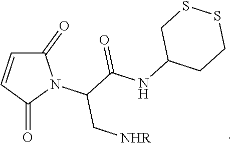

23. A thiol multiplex antibody drug conjugate (TM-ADC) comprising an antibody and from one to ten covalently attached Multiplexer Linking Assembly (MLA) Units, wherein each of said one to ten covalently attached Multiplexer Linking Assembly Units is attached to a cysteine thiol of a reduced interchain disulfide bond and/or to engineered cysteine residue in said antibody, or is attached to a modified functional group introduced into said antibody or is a heterocyclo from Click Chemistry of Diels-Alder Chemistry or other cycloaddition reaction, and wherein each of said covalently attached Multiplexer Linking Assembly Units has from two to four Drug Moieties attached thereto and an optional Partitioning Group (Y).

24. The TM-ADC of claim 28, wherein each of the MLAs has two Drug Moieties attached.

30. The TM-ADC of claim 29, wherein the Drug Moieties are from cytotoxic agents.

31. The TM-ADC of claim 28, having the formula (I^{Ab}): 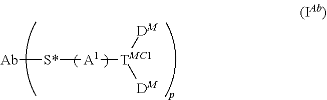
wherein:
Ab is from an antibody;
S* is selected from the group consisting of a sulfur atom of a cysteine residue of a reduced interchain disulfide bond, a sulfur atom from an engineered cysteine residue of said antibody, or an modified functional group introduced into said antibody or is a heterocyclo from Click Chemistry of Diels-Alder Chemistry or other cycloaddition reaction;
A¹ is a first Linking Group; wherein A¹ optionally comprises a Partitioning Group (Y) that is covalently attached to the first Linking Group, or is a divalent linking component of the first Linking Group;
bond or an independently selected second linking group T^{MC1} is a first Thiol Multiplexing Group;
wherein T^{MC1} and is attached to two Drug Moieties (D^{M}); and
subscript p is an integer of from 1 to 10.

25. The TM-ADC of claim 28, having the formula (II^{Ab}): 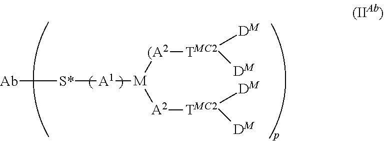
wherein:
4 Ab is from an antibody;
S* is selected from the group consisting of a sulfur atom of a cysteine residue of a reduced interchain disulfide bond, a sulfur atom from an engineered cysteine residue of said antibody, or an modified functional group introduced into said antibody or is a heterocyclo from Click Chemistry of Diels-Alder Chemistry or other cycloaddition reaction;
A¹ is a first Linking Group;
each A² is independently a bond or an independently selected second Linking Group;
each of A¹ and A² optionally comprises a Partitioning Group (Y) that is covalently attached to the first or second Linking Group, or is a divalent linking component of the first or second Linking Group;
M is a Multiplexing Group or a first Thiol Multiplexing Group (T^{MC1});
each T^{MC2} is a second Thiol Multiplexing Group in reduced form;
each D^{M} is a Drug Moiety attached to a sulfur atom of a T^{MC2} Group in reduced form; and
subscript p is an integer of from 1 to 10.

26. The TM-ADC of claim 28, having the formula (I^{Ab}a): 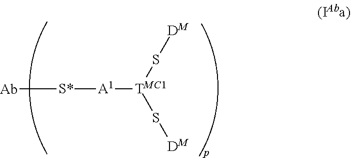
wherein:
Ab is from an antibody;
S* is selected from the group consisting of a sulfur atom of a cysteine residue of a reduced interchain disulfide bond, a sulfur atom from an engineered cysteine residue of said antibody, or an modified functional group introduced into said antibody or is a heterocyclo from Click Chemistry of Diels-Alder Chemistry or other cycloaddition reaction;
A¹ is a first Linking Group, optionally comprising a Partitioning Group (Y) that is covalently attached to the first or second Linking Group, or is a divalent linking component of the first or second Linking Group;
T^{MC1} is a Thiol Multiplexing Group;
each S is selected from the group consisting of a sulfur atom of T^{MC1};
each D^{M} is a Drug Moiety; and
subscript p is an integer of from 1 to 10.

27. The TM-ADC of claim 28, having the formula (I^{Ab}b): 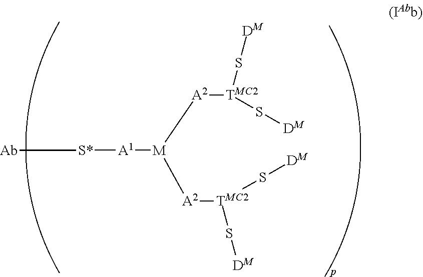
wherein:
Ab is from an antibody;
S* is selected from the group consisting of a sulfur atom of a cysteine residue of a reduced interchain disulfide bond, a sulfur atom from an engineered cysteine residue of said antibody, or an modified functional group introduced into said antibody or is a heterocyclo from Click Chemistry of Diels-Alder Chemistry or other cycloaddition reaction;
A¹ is a first Linking Group;
each A² is independently a bond or an independently selected second linking group;
each of A¹ and A² independently comprise 0 or 1 Partitioning Group (Y) that is covalently attached to the first or second Linking Group, or a divalent linking component of the first or second Linking Group;
M is a Multiplexing Group or a first Thiol Multiplexing Group (T^{MC1});
each T^{MC2} is independently a second Thiol Multiplexing Group;
each S is selected from the group consisting of a sulfur atom of the second Thiol Multiplexing Group (T^{MC2}); and
each D^{M} is a Drug Moiety.

28. The TM-ADC of any one of claim 32, 33, or 34, wherein A¹ has a formula selected from the group consisting of: 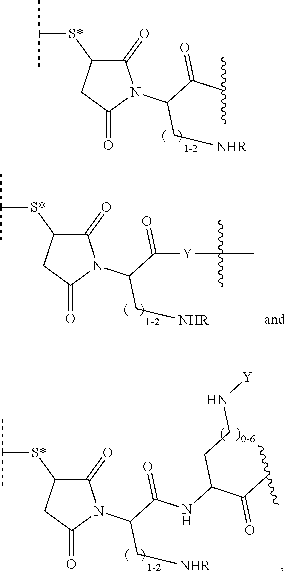
wherein the succinimide ring is optionally in hydrolyzed form; S* is selected from the group consisting of a sulfur atom of a reduced interchain disulfide bond or from an engineered cysteine unit of said antibody; R is selected from the group consisting of H and an amine protecting group; Y is a Partitioning Group; the wavy line indicates attachment to M; and the dashed line indicates attachment to the antibody.

29. The TM-ADC of any one of claim 32, 33, or 34, wherein A¹ has a formula selected from the group consisting of: 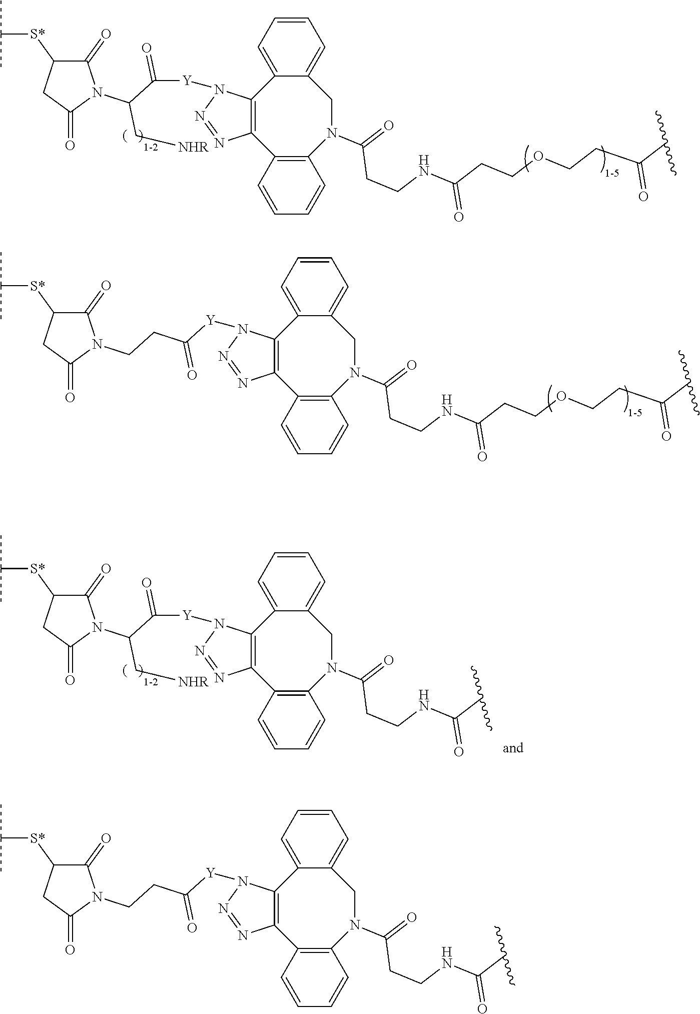
wherein the succinimide ring is optionally in hydrolyzed form; S* is selected from the group consisting of a sulfur atom of a reduced interchain disulfide linkage or from an engineered cysteine unit of said antibody; R is selected from the group consisting of H and an amine protecting group; Y is a Partitioning Group; the wavy line indicates covalent attachment to M; and the dashed line indicates covalent attachment to the antibody.

30. The TM-ADC of claim 32, wherein M is T^{MC1} and each T^{MC1} and T^{MC2} are independently selected from the group consisting of: 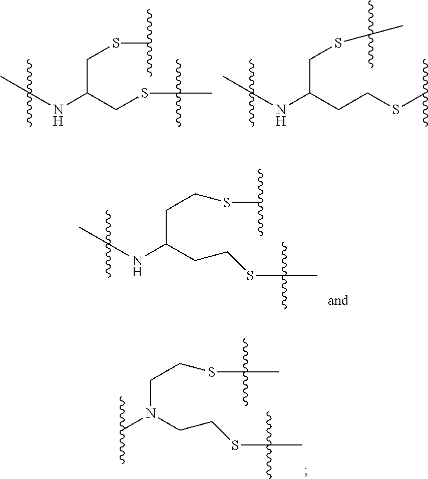 [SMILES: *NC(CS*)CS* ; *NC(CCS*)CS* ; *NC(CCS*)CCS* ; *SCCN(*)CCS*]
and each T^{MC2} is also attached to two Drug Moieties (D^{M}).

31. The TM-ADC of any one of claim 32, 33, or 34, wherein each of said D^{M} is selected from the group consisting of mc-VC-PAB-D, me-D, me-VC-D, MDPr-D, and MDPr-Lys(PEG)-D; wherein each D is a Drug Unit wherein the Drug Unit is from a chemotherapeutic nucleoside, an antimetabolite or a hydrophilic or a mildly hydrophobic chemotherapeutic characterized by a ClogP of 2.5 or less and/or a polar surface area of 80 angstroms squared or more.

32. The TM-ADC of claim 28 to 33, having the formula: 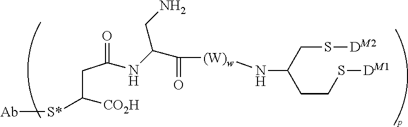
wherein
Ab is from an antibody;
each S* is a sulfur atom from said antibody;
each W is independently a natural or unnatural amino acid; wherein each W optionally comprises a Partitioning Group Partitioning Group (Y) that is covalently attached to W;
subscript w is 0, 1, 2 or 3;
D^{M1} is a first Drug Moiety;
D^{M2} is a second Drug Moiety; and
the subscript p is an integer of from 1 to 10.

40. The TM-ADC of claim 39, wherein subscript w is 0.

41. The TM-ADC of claim 39, wherein subscript w is 1 and the Partitioning Group (Y) is a PEG Unit comprised of 8 to 72, 8 to 36 or 8 to 24 or 12 to 24 contiguous polyethylene glycol subunits.

33. The TM-ADC of claim 39, wherein W_{w} is di- or tri-peptide residue.

34. The TM-ADC of claim 42, wherein each amino acid present in W_{w} is independently a residue of an amino acid selected from the group consisting of glycine, alanine, β-alanine and lysine.

35. The TM-ADC of claim 39, wherein D^{M1} and D^{M2} each include a first Drug Linker (D^{L1}) and a second Drug Linker (D^{L2}), respectively; wherein each D^{L1} and each D^{L2} and are independently selected from maleimido-caproyl (mc), maleimido-caproyl-valine-citrulline (mc-vc), maleimido-caproyl-valine-citrulline-paraaminobenzyloxycarbonyl (mc-vc-PABC) and maleimidodiaminopropionyl-valine-citrulline (MDPr-vc) in which the me or MDpr component have been converted to irs corresponding succinimide moiety that is optionally in hydrolyzed form.

36. The TM-ADC of claim 39, wherein each D^{M1} and each D^{M2} are independently incorporated the structure of free drug selected from a chemotherapeutic nucleoside, an antimetabolite or a hydrophilic or a mildly hydrophobic chemotherapeutic characterized by a ClogP of 2.5 or less and/or a polar surface area of 80 angstroms squared or more.
